(19) United States
(12) Patent Application Publication
(10) Pub. No.: US 2014/0199327 A1
(43) Pub. Date: Jul. 17, 2014

(54) METHODS OF PROMOTING DIFFERENTIATION

(71) Applicant: Genentech, Inc., South San Francisco, CA (US)

(72) Inventors: Vishva M. Dixit, Los Altos, CA (US); Dorothy M. French, San Carlos, CA (US); Heather L. Maecker, Palo Alto, CA (US); Samuel A. Williams, South San Francisco, CA (US)

(73) Assignee: Genentech, Inc., South San Francisco, CA (US)

(21) Appl. No.: 14/211,270

(22) Filed: Mar. 14, 2014

Related U.S. Application Data
(63) Continuation of application No. PCT/US2012/055539, filed on Sep. 14, 2012.
(60) Provisional application No. 61/535,336, filed on Sep. 15, 2011.

(51) Int. Cl. G01N 33/50

(52) U.S. Cl. CPC G01N 33/502
USPC 424/158.1; 435/6.13; 435/377

(57) ABSTRACT

Provided herein are methods of promoting cell fate change, particularly differentiation of tumor cells, by inhibition of USP1, UAF1, and/or ID (e.g., ID1, ID2, and/or ID3).

### CROSS REFERENCE TO RELATED APPLICATIONS

This is a continuation of International Application No. PCT/US2012/055539, filed Sep. 14, 2012 which claims priority under 35 USC §119 to U.S. Provisional Application No. 61/535,336, filed Sep. 15, 2011, the contents of which are incorporated by reference in its entirety.

### SEQUENCE LISTING

The instant application contains a Sequence Listing which has been submitted in ASCII format via EFS-Web and is hereby incorporated by reference in its entirety. Said ASCII copy, created on Mar. 10, 2014, is named P4745R1C1US.txt and is 49,143 bytes in size.

### FIELD OF THE INVENTION

Provided herein are methods of promoting cell fate change, particularly differentiation of tumor cells, by inhibition of USP1, UAF1, and/or ID (e.g., ID1, ID2, and/or ID3).

### BACKGROUND

Basic-helix-loop-helix (bHLH) transcription factors comprise the third-largest family of recognized transcription factors in the human genome (Tupler et al., 2001) and are essential regulators of development and differentiation through binding DNA elements termed E boxes (Massari and Murre, 2000). Class I bHLH homodimers are expressed broadly and promote expression of antiproliferative genes such as CDKN1A, CDKN2A, and CDKN2B (Yokota and Mori, 2002). Class II bHLH proteins show more restricted expression and form heterodimers with class I proteins to drive tissue-specific genes such as IGH@ and SP7/OSTERIX (Lassar et al., 1991; Weintraub et al., 1994). Through the combined induction of tissue-specific and antiproliferative genes, bHLH transcription factors serve as integrators of lineage commitment.

DNA binding of bHLH proteins is limited by heterodimerization with inhibitor of DNA-binding proteins, or IDs. The ID family consists of four members, ID1, ID2, ID3, and ID4 (Lasorella et al., 2001), with overlapping spatial and temporal expression profiles. All four IDs bind the various bHLH proteins with similar affinities to regulate gene expression (Prabhu et al., 1997). IDs are induced transcriptionally by myriad growth factors including bone morphogenic proteins, platelet-derived growth factor, epidermal growth factor, as well as by T cell receptor ligation (Yokota and Mori, 2002). ID1, ID2, and ID3, but not ID4, are subject to K48-linked polyubiquitination and subsequent degradation by the 26S proteasome. Consequently, IDs are short lived in most tissues (Bounpheng et al., 1999). The ubiquitously expressed APC/Cdh1 complex is an E3 ubiquitin ligase that governs ID stability and abundance (Lasorella et al., 2006), but ID proteins are stable in some contexts.

IDs are essential for mammalian development; disruption of two or more ID genes results in embryonic lethality (Lyden et al., 1999). In contrast, overexpression of ID proteins in transgenic mice produces fatal malignancies (Kim et al., 1999). Similarly, elevated ID protein levels are observed in a broad range of dedifferentiated primary human malignancies ranging from pancreatic carcinoma to neuroblastoma (Perk et al., 2005). An engineered ID-suppressing HLH protein was reported to differentiate neuroblastoma tumors (Ciarapica et al., 2009). Although ID proteins are scarce in normal adult differentiated tissues, they are abundant in proliferating tissues, including embryonic and adult stem cell populations, which suggests that IDs might maintain “stemness” (Yokota and Mori, 2002). More work is required to elucidate the role of ID genes in cancer stem cell biology.

### SUMMARY

Provided herein are methods of screening and/or identifying and methods of promoting a change in cell fate and/or cell cycle arrest using USP1 antagonists, UAF1 antagonists, and/or ID antagonists (e.g., ID1, ID2, and/or ID3).

Provided herein are methods of screening for and/or identifying an USP1 antagonist, UAF1 antagonist, and/or an ID antagonist which promotes a change in cell fate said method comprising: comparing (i) a reference cell fate, wherein the reference cell fate is the cell fate of a reference cell with (ii) a candidate cell fate, wherein the candidate cell fate is the cell fate of the reference cell in the presence of an USP1 candidate antagonist, UAF1 candidate antagonist, and/or an ID candidate antagonist, wherein the USP1 candidate antagonist binds USP1, wherein the UAF1 candidate antagonist binds UAF1, and/or the ID candidate antagonist binds ID, whereby a difference in cell fate between the reference cell fate and the candidate cell fate identifies the USP1 candidate antagonist and/or the ID candidate antagonist as promoting a change in cell fate.

Provided herein are also methods of screening for and/or identifying an USP1 antagonist, UAF1 antagonist, and/or an ID antagonist which induces cell cycle arrest said method comprising: (i) contacting a reference cell in the presence of an USP1 candidate antagonist, UAF1 candidate antagonist, and/or an ID candidate antagonist, wherein the USP1 candidate antagonist binds USP1, wherein the UAF1 candidate antagonist binds UAF1, and/or the ID candidate antagonist binds ID, whereby cell cycle arrest identifies the USP1 candidate antagonist and/or the ID candidate antagonist as inducing cell cycle arrest.

In some embodiments of any of the methods of screening, the USP1 candidate antagonist, UAF1 candidate antagonist, and/or the ID candidate antagonist is USP1 candidate antagonist. In some embodiments of any of the methods of screening, the USP1 candidate antagonist, UAF1 candidate antagonist, and/or the ID candidate antagonist is ID candidate antagonist. In some embodiments, the ID candidate antagonist is an ID1 candidate antagonist, an ID2 candidate antagonist, and/or an ID3 candidate antagonist. In some embodiments of any of the methods of screening, the USP1 candidate antagonist, UAF1 antagonist, and/or the ID candidate antagonist is UAF1 candidate antagonist.

In some embodiments of any of the methods of screening, the reference cell fate is a stem cell fate. In some embodiments, the stem cell fate is a mesenchymal stem cell fate. In some embodiments of any of the methods of screening, the candidate cell fate is an osteoblast cell fate, chondrocyte cell fate, or adipocyte cell fate. In some embodiments, the candidate cell fate is an osteoblast cell fate.

In some embodiments of any of the methods of screening, the USP1 candidate antagonist, UAF1 candidate antagonist, and/or the ID candidate antagonist is an antibody, binding polypeptide, binding small molecule, or polynucleotide.

Further provided herein are methods of promoting a change in cell fate of a cell comprising contacting the cell with an effective amount of USP1 antagonist, UAF1 antagonist, and/or an ID antagonist. Provided herein are also methods of inducing cell cycle arrest comprising contacting the cell with an effective amount of USP1 antagonist, UAF1 antagonist, and/or an ID antagonist. In some embodiments, the cell is a cell with a stem cell fate (e.g., mesenchymal stem cell fate).

Provided herein are methods of treating a disease or disorder comprising administering to an individual an effective amount of an USP1 antagonist, UAF1 antagonist, and/or an ID antagonist.

In some embodiments, the individual is selected for the treatment based upon elevated expression levels of one or more genes selected from the group consisting of CD90, CD105, CD106, USP1, UAF1, and ID (e.g., ID1, ID2, or ID3) (e.g., compared to an internal reference (e.g., CD144)) or the individual is not selected for the treatment based upon low expression levels of one or more genes selected from the group consisting of CD90, CD105, CD106, USP1, UAF1, and ID (e.g., ID1, ID2, or ID3) (e.g., compared to an internal reference (e.g., CD144)). In some embodiments, the individual is selected for the treatment based upon low expression levels of one or more genes selected from the group consisting of p21, RUNX2, OSTERIX, SPARC/OSTEONECTIN, SPP1/OSTEOPONTIN, BGLAP/OSTEOCALCIN, and alkaline phosphatase (ALP) (e.g., compared to an internal reference (e.g., CD144)) or the individual is not selected for the treatment based upon elevated expression levels of one or more genes selected from the group consisting of p21, RUNX2, OSTERIX, SPARC/OSTEONECTIN, SPP1/OSTEOPONTIN, BGLAP/OSTEOCALCIN, and alkaline phosphatase (ALP) (e.g., compared to an internal reference (e.g., CD144)).

In some embodiments, the individual is likely responsive to treatment based upon elevated expression levels of one or more genes selected from the group consisting of p21, RUNX2, OSTERIX, SPARC/OSTEONECTIN, SPP1/OSTEOPONTIN, BGLAP/OSTEOCALCIN, and alkaline phosphatase (ALP) (e.g., compared to an internal reference (e.g., CD144)) (e.g., from a time point at, during, or prior to the start of treatment to a later time point) or the individual is likely not responsive to treatment based upon reduced or no significant change of expression levels of one or more genes selected from the group consisting of p21, RUNX2, OSTERIX, SPARC/OSTEONECTIN, SPP1/OSTEOPONTIN, BGLAP/OSTEOCALCIN, and alkaline phosphatase (ALP) (e.g., compared to an internal reference (e.g., CD144)) (e.g., from a time point at, during, or prior to the start of treatment to a later time point).

In some embodiments of any of the methods, the USP1 antagonist, UAF1 antagonist, and/or an ID antagonist induces cell cycle arrest. In some embodiments of any of the methods, the USP1 antagonist, UAF1 antagonist, and/or an ID antagonist is capable of promoting a change in cell fate.

In some embodiments of any of the methods, promoting a change in cell fate is indicated by reduced expression levels of one or more genes selected from the group consisting of CD90, CD105, CD106, USP1, UAF1, and ID (e.g., ID1, ID2, or ID3) (e.g., compared to an internal reference (e.g., CD144)). In some embodiments of any of the methods, promoting a change in cell fate is indicated by elevated expression levels of one or more genes selected from the group consisting of p21, RUNX2, OSTERIX, SPARC/OSTEONECTIN, SPP1/OSTEOPONTIN, BGLAP/OSTEOCALCIN, and alkaline phosphatase (ALP). In some embodiments, expression levels of one or more genes is elevated compared to an internal reference (e.g., CD144).

In some embodiments of any of the methods, the disease or disorder comprises a cell with a stem cell fate (e.g., mesenchymal stem cell fate). In some embodiments of any of the methods, the cell expresses one or more genes selected from the group consisting of CD90, CD105, CD106, USP1, UAF1, and ID (e.g., ID1, ID2, or ID3). In some embodiments, expression levels of one or more genes is elevated compared to an internal reference (e.g., CD144). In some embodiments of any of the methods, the cell does not significantly express (e.g., does not express or expresses at low levels compared to an internal reference (e.g., CD144)) one or more genes selected from the group consisting of p21, RUNX2, OSTERIX, SPARC/OSTEONECTIN, SPP1/OSTEOPONTIN, BGLAP/OSTEOCALCIN, and alkaline phosphatase (ALP).

In some embodiments of any of the methods, the disease or disorder is cancer. In some embodiments, the cancer is osteosarcoma. In some embodiments, the cancer expresses one or more genes selected from the group consisting of CD90, CD105, CD106, USP1, UAF1, and ID (e.g., ID1, ID2, or ID3). In some embodiments, expression levels of one or more genes is elevated compared to an internal reference (e.g., CD144).

In some embodiments of any of the methods, the USP1 antagonist, UAF1 antagonist, and/or the ID antagonist is USP1 antagonist. In some embodiments of any of the methods, the USP1 antagonist, UAF1 antagonist, and/or the ID antagonist is ID antagonist. In some embodiments, wherein the ID antagonist is an ID1 antagonist, an ID2 antagonist, and/or an ID3 antagonist. In some embodiments of any of the methods, the USP1 antagonist, UAF1 antagonist, and/or the ID antagonist is UAF1 antagonist.

In some embodiments of any of the methods, the USP1 antagonist, UAF1 antagonist, and/or the ID antagonist is an antibody, binding polypeptide, binding small molecule, or polynucleotide. In some embodiments, the USP1 antagonist, UAF1 antagonist, and/or the ID antagonist is an antibody. In some embodiments, the antibody is a monoclonal antibody. In some embodiments, the antibody is a human, humanized, or chimeric antibody. In some embodiments, the antibody is an antibody fragment and the antibody fragment binds USP1, UAF, and/or an ID.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. USP1 Deubiquitinates and Stabilizes ID Proteins. (A) Western blot (WB) analysis of 293T cells transfected with vector only (CTL), wild-type USP1 (WT), or catalytically inactive USP1 C905. Cells were treated with 25 mg/ml cycloheximide (CHX) for the times indicated (left panel). ID2 was quantified by densitometry (right panel). (B) 293T cells were cotransfected with Flag-tagged ID1, ID2, ID3, or IkBa, and empty vector (CTL), wild-type USP1, or USP1 C905. Where indicated, cells were treated with 10 mM MG-132 for 4 hr. (C) Deubiquitination of ID2-Flag by USP1 or USP1 C90S and WDR48 in 293T cells cotransfected with HA-tagged ubiquitin. (D) USP1-Flag, USP1 C90S-Flag, WDR48-Flag, and ubiquitinated ID2-Flag were affinity purified separately from 293T extracts and then combined together for 6 hr in an in vitro deubiquitination assay. NEM, N-ethylmaleimide.

FIG. 2. Identification of USP1 as an ID2-Deubiquitinating Enzyme and Mapping of the USP1-ID2 Binding Interface. (A) Western blot (WB) analysis of 293T transfected with Flag-tagged deubiquitinases (DUBs) or an empty vector (−). Where indicated, cells were treated with 10 mM MG-132 for 4 hr. (B) Flag-tagged DUBs were immunoprecipitated (IP) from 293T cells cotransfected with ID2 and treated with 10 mM MG-132 for 6 hr. (C) USP1 mutants expressed in 293T cells were immunoprecipitated and blotted for co-expressed ID2. (D) Western blot analysis of endogenous ID2 in 293T cells transfected with wild-type (WT) or mutant USP1.

FIG. 3. USP1 Is Overexpressed in Osteosarcoma and Correlates with ID2 Protein Expression. (A) Box and whisker plots of USP1 mRNA expression in primary human bone biopsies from normal and diseased tissue. (B) Western blot (WB) analysis of USP1 and ID2 protein expression in primary human osteoblasts and osteosarcoma tumor samples. (C and D) RT-PCR quantification of USP1 (C) and ID2 (D) expression in the samples in (B). Bars represent the mean±SD of triplicate observations. (E and F) Immunohistochemical detection of ID2 in 293T cells transfected with an ID2 expression vector (top panel) or an ID2 shRNA (bottom panel) (E) or in a primary human osteosarcoma biopsy (F). (G) Immunohistochemical staining of USP1 and ID2 in serial sections from primary osteosarcoma tissue. Control staining was with an isotype-control antibody.

FIG. 4. USP1 Physically Engages and Stabilizes ID Proteins in Osteosarcoma. (A) Western blot (WB) analysis of U2-OS cells cotransfected with USP1 or control (CTL) shRNAs, plus either empty vector (CTL) or shRNA-resistant USP1 (wild-type [WT] or USP1 mutant C905). (B) Luciferase activity of U2-OS cells treated as in (A) and cotransfected with an E box-driven luciferase reporter. Bars represent the mean±SD of triplicate observations. (C) U2-OS cells were transfected with shRNAs and, where indicated, treated with 10 mM MG-132 for 4 hr. (D) U2-OS cells were cotransfected with ID2-Flag, HA-ubiquitin, and either CTL or USP1 shRNAs. Where indicated, cells were treated with 10 mMMG-132 for 4 hr. ID2-Flag was immunoprecipitated from SDS/heat-denatured cell lysates. (E and F) USP1 (E) or ID2 (F) was immunoprecipitated from U2-OS cells. Control immunoprecipitations were with nonspecific IgG. Asterisk (*) denotes a band of unknown identity recognized by the anti-ID2 antibody.

FIG. 5. USP1 Regulates ID Proteins in Multiple Osteosarcoma Cell Lines. (A) Western blot (WB) analysis of cultured primary human osteoblasts and human osteosarcoma cell lines. (B) Osteosarcoma cell lines were treated with 10 mM MG-132 for 4 hr. (C) Osteosarcoma cell lines were transfected with control (CTL) or USP1 shRNAs. (D) Osteosarcoma cells were transfected with empty vector or WDR48, or were treated with 10 mM MG-132 for 4 hr. (E) USP1 was immunoprecipitated from HOS cells. Control immunoprecipitations were with nonspecific IgG. (F) Analysis of USP1^{+/+} (WT) and USP1^{-/-} DT40 cells. (G) Real-time RT-PCR quantification of USP1 mRNA in WT and USP1^{-/-} DT40 cells. Bars represent the mean±s.d. of triplicate observations. (H) WT and USP1^{-/-} DT40 cells were treated with 10 mM MG-132 for 2 hr. (I) USP1^{-/-} DT40 cells were transfected with empty vector (CTL), USP1 wild-type (WT), or USP1 C90S and compared to USP1^{-/-} DT40 cells. Un, untransfected.

FIG. 6. USP1 Regulates Cell Cycling via ID Proteins in Osteosarcoma. (A) Western blot (WB) analysis of U2-OS cells treated as in FIG. 4A. (B) Outgrowth of U2-OS cells treated as in (A) was enumerated after 5 days of culture. (C) Cell cycle status of propidium iodide-stained U2-OS cells treated as in (A). (D) U2-OS cells transfected with indicated shRNAs and control or CDKN1A/p21 siRNAs. (E) Quantification of cells in S phase in cells treated as in (D). (F) U2-OS cells transfected with indicated shRNAs and shRNA-resistant USP1 (shRes USP1), ID1, ID2, and ID3, or control expression vectors. (G) Quantification of cells in S phase in U2-OS cells treated as in (F). Bars represent the mean±SD of triplicate observations.

FIG. 7. USP1 Regulates Proliferation and Cell-Cycle Arrest via ID Proteins. (A) U2-OS cells were transfected with control (CTL) or USP1 shRNAs for 3 days, plated at equivalent density, and viable cells were counted on subsequent days. (B) U2-OS cells cotransfected with shRNAs and, where indicated, shRNA-resistant USP1 (wild-type or mutant). (C) Percentage of cells in (B) in S-phase of the cell cycle. (D) Osteosarcoma cells were transfected with shRNAs and cells enumerated at day 8. (E) DNA content of U2-OS cells treated as in (A) and stained with propidium iodide (PI). (F) U2-OS cells were transfected with indicated shRNAs and with control or p21 siRNAs. (G) Cells in (F) were stained with propidium iodide and analyzed by flow cytometry. Bars represent the mean percentage of cells in S-phase. (H) U2-OS cells were transfected with the indicated shRNAs. (I-K) Cells in (H) were assessed by real-time RT-PCR (I) and flow cytometry after PI staining (J, K). (L) U2-OS cells were transfected with shRNAs and control or p53 siRNAs. Where indicated, cells were treated with 10 mM etoposide for 1 hr. Bars represent the mean±s.d. of triplicate observations.

FIG. 8. USP1 Promotes Retention of Stem Cell Identity in Osteosarcoma. (A) Western blot (WB) analysis of U2-OS cells transfected with CTL or USP1 shRNAs. (B) Cells in (A) were stained and analyzed by fluorescence microscopy. (C) Immunohistochemical staining for USP1 or ID2 in xenografts of 143B cells with doxycycline (DOX)-inducible shUSP1. (D) Quantification of tumor volume of 143B xenografts as described in (C). Bars represent the mean±SD of ten xenografts. (E and F) RT-PCR quantification of USP1, ID2, OSTEONECTIN (ON), RUNX2 (RX2), OSTERIX (OSX), and OSTEOPONTIN (OP) mRNA levels (E) and ALP activity (F) from 143B xenografts in (C). Bars represent the mean±SD of triplicate observations. (G) Representative xenograft tumors from (C) were stained with hematoxylin and eosin (H&E) or trichrome stain. Scale bars, 100 mm.

FIG. 9. Depletion of USP1 Induces Loss of Stem Markers and Initiates Osteogenic Program in Osteosarcoma Cell Lines. (A) Osteosarcoma cells were serially transfected with control (CTL), USP1, or ID shRNAs. Surface expression of the indicated mesenchymal stem cell markers was determined by flow cytometry after 11 days. (B) Cells in (A) were analyzed by real time RT-PCR for RUNX2, OSTERIX (OSX), and OSTEONECTIN gene expression. (C) Cells in (A) were assessed for alkaline phosphatase activity by p-nitrophenol-phosphate (pNPP) cleavage. (D) Western blot (WB) analysis of 143B cells transduced with doxycycline-inducible CTL or USP1 shRNAs. Where indicated, cells were treated with 3 mg/ml doxycycline (DOX) for 4 days. (E) Bright field and dark field microscopy of OSTEOCALCIN gene expression by in situ hybridization in sections of 143B shUSP1 xenograft tumors following 5 days of doxycyline treatment. Scale bars, 100 mm. (F) Real-time RT-PCR analysis of USP1 gene expression in control and USP1 shRNA-containing 143B xenograft tumors. Bars represent the mean±s.d. of triplicate observations.

FIG. 10. USP1 and IDs Regulate Mesenchymal Stem Cell Differentiation. (A) Western blot (WB) analysis of hMSCs grown in osteogenic differentiation medium (ODM), or in nondifferentiating medium (Un). (B) hMSCs were transduced with ID2, USP1 wild-type (WT), USP1 C905, or empty vector (CTL) and cultured in ODM for 9 days. (C and D) hMSCs in (B) were assessed for ALP activity (C) and OSTEONECTIN, RUNX2, and OSTERIX mRNA (D). Bars represent the mean±SD of triplicate observations. (E) hMSCs in (B) stained with alizarin red to visualize calcium deposition. Scale bars, 100 mm. (F) Enumeration of hMSCs in (B) after the indicated number of days of culture. Bars represent the mean±SD of triplicate observations.

FIG. 11. USP1 Induces ID-Dependent Transformation of NIH 3T3 Cells. (A) Western blot (WB) analysis of NIH 3T3 cells transduced with ID2, USP1 wild-type (WT), USP1 C905, or an empty control vector. (B) Cells in (A) were grown in soft agar, and colonies were enumerated. Bars represent the mean±s.d. of triplicate observations. (C) Representative colonies formed by NIH 3T3 cells transduced with control (CTL), ID2, USP1 wild-type (WT), or USP1 C905. Scale bars, 100 mm. (D) NIH 3T3 cells in (A) were implanted subcutaneously in C.B-17 SCID.bg mice (top panel) or NCr nude mice (bottom panel) and tumor volume was monitored. Data points represent the mean±s.d. of ten mice. (E) C.B-17 SCID.bg (top panels) and NCr nude mice (bottom panels) from (A) at the end of the study. (F) Empty vector (CTL)- or USP1-transduced NIH 3T3 cells were sequentially transduced with control (CTL) or ID shRNAs. (G) Cells in (F) were grown in soft agar, and colonies were enumerated. Bars represent the mean±s.d. of triplicate observations.

FIG. 12. USP1 Is Required for Normal Skeletogenesis. (A) Microcomputed tomography of 12-day-old USP1^{+/+} (WT) and USP1^{-/-} mice (top) and femurs (bottom). (B and C) Mean bone mineralized density (BMD) (B) and mineralized bone volume (Minz. Vol.) (C) of mice in (A). Bars represent the mean±SD of four femurs of each genotype. (D) Western blot (WB) analysis of femoral metaphyses from E18.5 USP1^{+/+} (WT) and USP1^{-/-} mice. (E) BALP in the sera of E18.5 USP1^{+/+} (WT) and USP1^{-/-} embryos. Bars represent the mean±SD of four embryos of each genotype.

FIG. 13. USP1 Is Required for Normal Mouse Skeletogenesis. (A) USP1 targeting strategy to delete exon 3, which encodes the catalytic cysteine of USP1. Yellow boxes represent exons. (B) Micro-computed tomography of E18.5 USP1^{+/+} (WT) and USP1^{-/-} embryos. (C) Mineralized bone volume (Minz. Vol.) of mice in (b). Bars represent the mean±s.d. of 3 mice of each genotype. (D) Hematoxylin and eosin (H&E) stained sections of P12 USP1^{+/+} (WT) and USP1^{-/-} femurs. Scale bars, 100 mm. (E) Osteoid area per length of spicule in P12 USP1^{+/+} (WT) and USP1^{-/-} femurs. Bars represent the mean±s.d. of 3 mice of each genotype. (F) H&E, trichrome, and Von Kossa stains of P12 USP1^{+/+} (WT) and USP1^{-/-} femoral metaphyses. Scale bars, 100 mm. (G) TRAP labeling of resident osteoclasts in P12 USP1^{+/+} (WT) and USP1^{-/-} femurs. Scale bars, 100 mm. (H) Enumeration of TRAP-positive cells in P12 USP1^{+/+} (WT) and USP1^{-/-} femur sections. (I) Creatinine-normalized deoxypyridinoline (DPD) levels in E18.5 amniotic fluid. (J) USP1 and ID2 expression in P12 USP1^{+/+} (WT) and USP1^{-/-} femoral metaphyses. Scale bars, 100 mm.

### DETAILED DESCRIPTION

### I. Definitions

The terms “ubiquitin specific peptidase 1,” “deubiquitinating enzyme 1,” and “USP1” refer herein to a native sequence USP1 polypeptide, polypeptide variants and fragments of a native sequence polypeptide and polypeptide variants (which are further defined herein). The USP polypeptide described herein may be that which is isolated from a variety of sources, such as from human tissue types or from another source, or prepared by recombinant or synthetic methods.

A “native sequence USP1 polypeptide” comprises a polypeptide having the same amino acid sequence as the corresponding USP1 polypeptide derived from nature. In one embodiment, a native sequence USP1 polypeptide comprises the amino acid sequence of SEQ ID NO:1.

“USP1 polypeptide variant”, or variations thereof, means an USP1 polypeptide, generally an active USP1 polypeptide, as defined herein having at least about 80% amino acid sequence identity with any of the native sequence USP1 polypeptide sequences as disclosed herein. Such USP1 polypeptide variants include, for instance, USP1 polypeptides wherein one or more amino acid residues are added, or deleted, at the N- or C-terminus of a native amino acid sequence. Ordinarily, a USP1 polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a native sequence USP1 polypeptide sequence as disclosed herein. Ordinarily, USP1 variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 amino acids in length, or more. Optionally, USP1 variant polypeptides will have no more than one conservative amino acid substitution as compared to a native USP1 polypeptide sequence, alternatively no more than 2, 3, 4, 5, 6, 7, 8, 9, or 10 conservative amino acid substitution as compared to the native USP1 polypeptide sequence.

The term “USP 1 antagonist” as defined herein is any molecule that partially or fully blocks, inhibits, or neutralizes a biological activity mediated by a native sequence USP 1. In certain embodiments such antagonist binds to USP1. According to one embodiment, the antagonist is a polypeptide. According to another embodiment, the antagonist is an anti-USP1 antibody. According to another embodiment, the antagonist is a small molecule antagonist. According to another embodiment, the antagonist is a polynucleotide antagonist.

The terms “WD repeat domain 48,” “USP1-associated factor 1,” and “UAF1” refer herein to a native sequence UAF1 polypeptide, polypeptide variants and fragments of a native sequence polypeptide and polypeptide variants (which are further defined herein). The UAF1 polypeptide described herein may be that which is isolated from a variety of sources, such as from human tissue types or from another source, or prepared by recombinant or synthetic methods.

A “native sequence UAF1 polypeptide” comprises a polypeptide having the same amino acid sequence as the corresponding UAF1 polypeptide derived from nature. In one embodiment, a native sequence UAF1 polypeptide comprises the amino acid sequence of SEQ ID NO:40.

“UAF1 polypeptide variant”, or variations thereof, means an UAF1 polypeptide, generally an active UAF1 polypeptide, as defined herein having at least about 80% amino acid sequence identity with any of the native sequence UAF1 polypeptide sequences as disclosed herein. Such UAF1 polypeptide variants include, for instance, UAF1 polypeptides wherein one or more amino acid residues are added, or deleted, at the N- or C-terminus of a native amino acid sequence. Ordinarily, a UAF1 polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a native sequence UAF1 polypeptide sequence as disclosed herein. Ordinarily, UAF1 variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 amino acids in length, or more. Optionally, UAF1 variant polypeptides will have no more than one conservative amino acid substitution as compared to a native UAF1 polypeptide sequence, alternatively no more than 2, 3, 4, 5, 6, 7, 8, 9, or 10 conservative amino acid substitution as compared to the native UAF1 polypeptide sequence.

The term “UAF1 antagonist” as defined herein is any molecule that partially or fully blocks, inhibits, or neutralizes a biological activity mediated by a native sequence UAF1. In certain embodiments such antagonist binds to UAF1. According to one embodiment, the antagonist is a polypeptide. According to another embodiment, the antagonist is an anti-UAF1 antibody. According to another embodiment, the antagonist is a small molecule antagonist. According to another embodiment, the antagonist is a polynucleotide antagonist.

The terms “inhibitor of DNA binding” and “ID” refer herein to a native sequence ID polypeptide, polypeptide variants and fragments of a native sequence polypeptide and polypeptide variants (which are further defined herein). The ID polypeptide described herein may be that which is isolated from a variety of sources, such as from human tissue types or from another source, or prepared by recombinant or synthetic methods.

A “native sequence ID polypeptide” comprises a polypeptide having the same amino acid sequence as the corresponding ID polypeptide derived from nature. In some embodiments of any of the native sequence ID polypeptides, the native sequence ID polypeptide includes a native sequence ID1 isoform a polypeptide of SEQ ID NO:2. In some embodiments of any of the native sequence ID polypeptides, the native sequence ID polypeptide includes a native sequence ID1 isoform b polypeptide of SEQ ID NO:3. In some embodiments of any of the native sequence ID polypeptides, the native sequence ID polypeptide includes a native sequence ID2 polypeptide of SEQ ID NO:4. In some embodiments of any of the native sequence ID polypeptides, the native sequence ID polypeptide includes a native sequence ID3 polypeptide of SEQ ID NO:5.

“ID polypeptide variant”, or variations thereof, means an ID polypeptide, generally an active ID polypeptide, as defined herein having at least about 80% amino acid sequence identity with any of the native sequence ID polypeptide sequences as disclosed herein. Such ID polypeptide variants include, for instance, ID polypeptides wherein one or more amino acid residues are added, or deleted, at the N- or C-terminus of a native amino acid sequence. Ordinarily, an ID polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a native sequence ID polypeptide sequence as disclosed herein. Ordinarily, ID variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 amino acids in length, or more. Optionally, ID variant polypeptides will have no more than one conservative amino acid substitution as compared to a native ID polypeptide sequence, alternatively no more than 2, 3, 4, 5, 6, 7, 8, 9, or 10 conservative amino acid substitution as compared to the native ID polypeptide sequence. In some embodiments of any of the ID polypeptide variants, the ID polypeptide variant includes an ID1 polypeptide variant. In some embodiments of any of the ID polypeptide variants, the ID polypeptide variant includes an ID2 polypeptide variant. In some embodiments of any of the ID polypeptide variants, the ID polypeptide variant includes an ID3 polypeptide variant.

The term “ID antagonist” as defined herein is any molecule that partially or fully blocks, inhibits, or neutralizes a biological activity mediated by a native sequence ID. In certain embodiments such antagonist binds to ID. According to one embodiment, the antagonist is a polypeptide. According to another embodiment, the antagonists is an anti-ID antibody. According to another embodiment, the antagonist is a small molecule antagonist. According to another embodiment, the antagonist is a polynucleotide antagonist. In some embodiments of any of the ID antagonists, the ID antagonist is an ID1 antagonist. In some embodiments of any of the ID antagonists, the ID antagonist is an ID2 antagonist. In some embodiments of any of the ID antagonists, the ID antagonist is an ID3 antagonist.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

The term “small molecule” refers to any molecule with a molecular weight of about 2000 daltons or less, preferably of about 500 daltons or less.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The terms “anti-USP1 antibody” and “an antibody that binds to USP1” refer to an antibody that is capable of binding USP1 with sufficient affinity such that the antibody is useful as a diagnostic and/or therapeutic agent in targeting USP1. In one embodiment, the extent of binding of an anti-USP1 antibody to an unrelated, non-USP1 protein is less than about 10% of the binding of the antibody to USP1 as measured, e.g., by a radioimmunoassay (RIA). In certain embodiments, an anti-USP1 antibody binds to an epitope of USP1 that is conserved among USP1 from different species.

The terms “anti-ID antibody” and “an antibody that binds to ID” refer to an antibody that is capable of binding ID with sufficient affinity such that the antibody is useful as a diagnostic and/or therapeutic agent in targeting ID. In one embodiment, the extent of binding of an anti-ID antibody to an unrelated, non-ID protein is less than about 10% of the binding of the antibody to ID as measured, e.g., by a radioimmunoassay (RIA). In certain embodiments, an anti-ID antibody binds to an epitope of ID that is conserved among ID from different species. In some embodiments of any of the anti-ID antibodies, the ID antibody is an anti-ID 1 antibody. In some embodiments of any of the anti-ID antibodies, the ID antibody is an anti-ID2 antibody. In some embodiments of any of the anti-ID antibodies, the ID antibody is an anti-ID3 antibody.

A “blocking” antibody or an “antagonist” antibody is one which inhibits or reduces biological activity of the antigen it binds. Preferred blocking antibodies or antagonist antibodies substantially or completely inhibit the biological activity of the antigen.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called a, 6, c, y, and μ, respectively.

The terms “full length antibody,” “intact antibody,” and “whole antibody” are used herein interchangeably to refer to an antibody having a structure substantially similar to a native antibody structure or having heavy chains that contain an Fc region as defined herein.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “immunoconjugate” is an antibody conjugated to one or more heterologous molecule(s), including but not limited to a cytotoxic agent.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

An “effective amount” of an agent refers to an amount effective, at dosages and for periods of time necessary, to achieve the desired therapeutic or prophylactic result.

A “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “anti-cancer therapy” refers to a therapy useful in treating cancer. Examples of anti-cancer therapeutic agents include, but are limited to, e.g., chemotherapeutic agents, growth inhibitory agents, cytotoxic agents, agents used in radiation therapy, anti-angiogenesis agents, apoptotic agents, anti-tubulin agents, and other agents to treat cancer, anti-CD20 antibodies, platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets PDGFR-beta, BlyS, APRIL, BCMA receptor(s), TRAIL/Apo2, and other bioactive and organic chemical agents, etc. Combinations thereof are also included in the invention.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” refers to a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releaseing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

A “growth inhibitory agent” when used herein refers to a compound or composition which inhibits growth of a cell (e.g., a cell whose growth is dependent upon USP1 expression either in vitro or in vivo). Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated, the presence or size of metastases, or the size of the primary tumor.

The term “package insert” is used to refer to instructions customarily included in commercial packages of therapeutic products, that contain information about the indications, usage, dosage, administration, combination therapy, contraindications and/or warnings concerning the use of such therapeutic products.

It is understood that aspect and embodiments of the invention described herein include “consisting” and/or “consisting essentially of” aspects and embodiments. As used herein, the singular form “a”, “an”, and “the” includes plural references unless indicated otherwise.

### II. Methods and Uses

Provided herein are methods utilizing an USP1 antagonist, UAF1 antagonist, and/or an ID antagonist. For example, provided herein are methods of promoting a change in cell fate of a cell comprising contacting the cell with an effective amount of USP1 antagonist, UAF1 antagonist, and/or an ID antagonist. Provided herein are also methods of inducing cell cycle arrest comprising contacting the cell with an effective amount of USP1 antagonist, UAF1 antagonist, and/or an ID antagonist. In some embodiments, the cell is a cell with a stem cell fate (e.g., mesenchymal stem cell fate).

Provided herein are methods of treating a disease or disorder comprising administering to an individual an effective amount of an USP1 antagonist, UAF1 antagonist, and/or an ID antagonist.

Provided herein are methods of inducing bone growth comprising administering to an individual an effective amount of an USP1 antagonist, UAF1 antagonist, and/or an ID antagonist.

Provided herein are methods of sensitizing and/or resensitizing an individual to a chemotherapeutic agent comprising administering to an individual an effective amount of an USP1 antagonist, UAF1 antagonist, and/or an ID antagonist.

Provided herein are methods of inducing and/or promoting EMT comprising administering to an individual an effective amount of an USP1 antagonist, UAF1 antagonist, and/or an ID antagonist.

Provided herein are methods of treating cancer resistant to chemotherapeutic agent comprising administering to an individual an effective amount of an USP1 antagonist, UAF1 antagonist, and/or an ID antagonist.

In some embodiments, the individual is selected for the treatment based upon elevated expression levels of one or more genes selected from the group consisting of CD90, CD105, CD106, USP1, UAF1, and ID (e.g., ID1, ID2, or ID3) (e.g., compared to a reference value and/or to an internal reference (e.g., CD144)) or the individual is not selected for the treatment based upon low expression levels of one or more genes selected from the group consisting of CD90, CD105, CD106, USP1, UAF1, and ID (e.g., ID1, ID2, or ID3) (e.g., compared to a reference value and/or an internal reference (e.g., CD144)). In some embodiments, the individual is selected for the treatment based upon low expression levels of one or more genes selected from the group consisting of p21, RUNX2, OSTERIX, SPARC/OSTEONECTIN, SPP1/OSTEOPONTIN, BGLAP/OSTEOCALCIN, and alkaline phosphatase (ALP) (e.g., compared to a reference value and/or an internal reference (e.g., CD144)) or the individual is not selected for the treatment based upon elevated expression levels of one or more genes selected from the group consisting of p21, RUNX2, OSTERIX, SPARC/OSTEONECTIN, SPP1/OSTEOPONTIN, BGLAP/OSTEOCALCIN, and alkaline phosphatase (ALP) (e.g., compared to a reference value and/or an internal reference (e.g., CD144)).

In some embodiments, the individual is likely responsive to treatment based upon elevated expression levels of one or more genes selected from the group consisting of p21, RUNX2, OSTERIX, SPARC/OSTEONECTIN, SPP1/OSTEOPONTIN, BGLAP/OSTEOCALCIN, and alkaline phosphatase (ALP) (e.g., compared to a reference value and/or an internal reference (e.g., CD144)) (e.g., from a time point at, during, or prior to the start of treatment to a later time point) or the individual is likely not responsive to treatment based upon reduced or no significant change of expression levels of one or more genes selected from the group consisting of p21, RUNX2, OSTERIX, SPARC/OSTEONECTIN, SPP1/OSTEOPONTIN, BGLAP/OSTEOCALCIN, and alkaline phosphatase (ALP) (e.g., compared to a reference value and/or an internal reference (e.g., CD144)) (e.g., from a time point at, during, or prior to the start of treatment to a later time point).

In some embodiments of any of the methods, the USP1 antagonist, UAF1 antagonist, and/or an ID antagonist induces cell cycle arrest. In some embodiments of any of the methods, the USP1 antagonist, UAF1 antagonist, and/or an ID antagonist is capable of promoting a change in cell fate. In some embodiments of any of the methods, the USP1 antagonist, UAF1 antagonist, and/or an ID antagonist is capable of promoting and/or inducing EMT.

In some embodiments of any of the methods, promoting a change in cell fate is indicated by reduced expression levels of one or more genes selected from the group consisting of CD90, CD105, CD106, USP1, UAF1, and ID (e.g., ID1, ID2, or ID3) (e.g., compared to a reference value and/or an internal reference (e.g., CD144)). In some embodiments of any of the methods, promoting a change in cell fate is indicated by elevated expression levels of one or more genes selected from the group consisting of p21, RUNX2, OSTERIX, SPARC/OSTEONECTIN, SPP1/OSTEOPONTIN, BGLAP/OSTEOCALCIN, and alkaline phosphatase (ALP). In some embodiments, expression levels of one or more genes is elevated compared to an internal reference (e.g., CD144).

In some embodiments of any of the methods, the disease or disorder comprises a cell with a stem cell fate (e.g., mesenchymal stem cell fate). In some embodiments of any of the methods, the cell expresses one or more genes selected from the group consisting of CD90, CD105, CD106, USP1, UAF1, and ID (e.g., ID1, ID2, or ID3). In some embodiments, expression levels of one or more genes is elevated compared to an internal reference (e.g., CD144). In some embodiments of any of the methods, the cell does not significantly express (e.g., does not express or expresses at low levels compared to an internal reference (e.g., CD144)) one or more genes selected from the group consisting of p21, RUNX2, OSTERIX, SPARC/OSTEONECTIN, SPP1/OSTEOPONTIN, BGLAP/OSTEOCALCIN, and alkaline phosphatase (ALP).

In some embodiments of any of the methods, the disease or disorder is cancer. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer. In some embodiments, the cancer is osteosarcoma. In some embodiments, the cancer is not Ewing's sarcoma. In some embodiments, the cancer is breast cancer. In some embodiments, the cancer is not breast cancer. In some embodiments, the cancer expresses (has been shown to express) one or more genes selected from the group consisting of CD90, CD105, CD106, USP1, UAF1, and ID (e.g., ID1, ID2, or ID3). In some embodiments, expression levels of one or more genes is elevated compared to an internal reference (e.g., CD144). In some embodiments, the cancer is refractory to treatment with one or more chemotherapeutic agent. In some embodiments, the cancer has been previously treated with a chemotherapeutic agent.

In some embodiments of any of the methods, the USP1 antagonist, UAF1 antagonist, and/or the ID antagonist is USP1 antagonist. In some embodiments of any of the methods, the USP1 antagonist, UAF1 antagonist, and/or the ID antagonist is ID antagonist. In some embodiments, wherein the ID antagonist is an ID1 antagonist, an ID2 antagonist, and/or an ID3 antagonist. In some embodiments of any of the methods, the USP1 antagonist, UAF1 antagonist, and/or the ID antagonist is UAF1 antagonist.

In some embodiments of any of the methods, the USP1 antagonist, UAF1 antagonist, and/or the ID antagonist is an antibody, binding polypeptide, binding small molecule, or polynucleotide. In some embodiments, the USP1 antagonist, UAF1 antagonist, and/or the ID antagonist is an antibody. In some embodiments, the antibody is a monoclonal antibody. In some embodiments, the antibody is a human, humanized, or chimeric antibody. In some embodiments, the antibody is an antibody fragment and the antibody fragment binds USP1, UAF, and/or an ID.

An “individual” according to any of the above embodiments may be a human.

In a further aspect, the invention provides a method for treating a cancer. In one embodiment, the method comprises administering to an individual having such cancer an effective amount of a USP1 antagonist, a UAF1 antagonist and/or an ID antagonist. In one such embodiment, the method further comprises administering to the individual an effective amount of at least one additional therapeutic agent, as described below. An “individual” according to any of the above embodiments may be a human.

In a further aspect, the invention provides a method for inducing and/or promoting EMT, promoting bone growth, inhibiting cell proliferation, promoting cell cycle arrest or promoting a change in a cell fate in an individual. In one embodiment, the method comprises administering to the individual an effective amount of a USP1 antagonist, UAF1 antagonist and/or ID antagonist to induce and/or promote EMT, promote bone growth, inhibit cell proliferation, promote cell cycle arrest or promote a change in a cell fate. In one embodiment, an “individual” is a human. In some embodiments, the individual has cancer. In some embodiments, the cancer is refractory or resistant to treatment with a chemotherapeutic agent.

In a further aspect, the invention provides pharmaceutical formulations comprising any of the USP1 antagonist, UAF1 antagonist and/or ID antagonist provided herein, e.g., for use in any of the above therapeutic methods. In one embodiment, a pharmaceutical formulation comprises any of the USP1 antagonist, UAF1 antagonist and/or ID antagonist provided herein and a pharmaceutically acceptable carrier. In another embodiment, a pharmaceutical formulation comprises any of the USP1 antagonist, UAF1 antagonist and/or ID antagonist provided herein and at least one additional therapeutic agent, e.g., as described below.

Antagonists of the invention can be used either alone or in combination with other agents in a therapy. For instance, an antibody of the invention may be co-administered with at least one additional therapeutic agent. In certain embodiments, an additional therapeutic agent is a chemotherapeutic agent.

Such combination therapies noted above encompass combined administration (where two or more therapeutic agents are included in the same or separate formulations), and separate administration, in which case, administration of the antagonist of the invention can occur prior to, simultaneously, and/or following, administration of the additional therapeutic agent and/or adjuvant. Antagonists of the invention can also be used in combination with radiation therapy.

An antagonist (e.g., an antibody) of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antagonists (e.g., antibodies) of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antagonist present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. An exemplary dosing regimen comprises administering. However, other dosage regimens may be useful. The progress of this therapy is easily monitored by conventional techniques and assays.

It is understood that any of the above formulations or therapeutic methods may be carried out using an immunoconjugate of the invention in place of or in addition to the USP1 antagonist, UAF1 antagonist, and/or an ID antagonist.

### III. Therapeutic Compositions

Provided herein are USP1 antagonists, UAF1 antagonists, and/or ID antagonists (e.g., ID1, ID2, and/or ID3) useful in the methods described herein. In some embodiments, the USP1 antagonists, UAF1 antagonists, and/or ID antagonists (e.g., ID1, ID2, and/or ID3) are an antibody, binding polypeptide, binding small molecule, or polynucleotide.

### A. Antibodies

In one aspect, provided herein isolated antibodies that bind to USP1, UAF1, and/or ID (e.g., ID1, ID2, or ID3). In any of the above embodiments, an antibody is humanized.

In some embodiments, the antibody is an USP1 antagonist. In some embodiments, the antibody is an UAF1 antagonist. In some embodiments, the antibody is an ID1 antagonist. In some embodiments, the antibody is an ID2 antagonist. In some embodiments, the antibody is an ID3 antagonist. In some embodiments, the antibody is capable of inhibiting more than one ID (e.g., two IDs, three IDs, or four IDs). In some embodiments, the antibody inhibits interaction of USP1 with UAF1. In some embodiments, the antibody blocks deubiquitination of ID. In some embodiment, the antibody inhibits interaction of ID with bHLH.

In some embodiments, the antibody is an USP1 antagonist and the USP1 antagonist is an antibody disclosed in US Patent Publication No. 2010/0330599, the contents of which are incorporated by referenced herein in its entirety. In some embodiments, the antibody is an ID 1 antagonist and the ID 1 antagonist is an antibody disclosed in U.S. Pat. No. 7,517,663, the contents of which are incorporated by referenced herein in its entirety. In some embodiments, the antibody is an ID3 antagonist and the ID3 antagonist is an antibody disclosed in U.S. Pat. No. 7,629,131, the contents of which are incorporated by referenced herein in its entirety.

In some embodiments, the anti-ID3 antibody comprises a variable light chain sequence comprising: QVLTQTPSPVSAAVGGTVTINCQASQSIYNDNDLAWFQQKPG QPPKLLIYDASTLTSGVPSRFKGSGSGTQFTLTISDLDCDDAATYYCAARYSGNIYGF (SEQ ID NO: 41) and/or a variable heavy chain sequence comprising: QSVEESGGRLVTPGTPLTLTCTVSGIDLSSYAMSW VRQAPGKGLEWIGVIFPSNNVYYASWAKGRFTISKTSTTVDLKITSPTTEDTATYFCASMGAFDSW GPGTLVTVSSG (SEQ ID NO: 42). In some embodiments, the anti-ID3 antibody comprises a variable light chain sequence comprising: AVLTQTPSPVSAAVGGTVSISCQSSQSVWNNNWLSWFQQKPGQPPKLLIY ETSKLESGVPSRFKGSGSGTQFTLTISDVQCDDAATYYCLGGYWTTSDNNVFGGGTEVVVK (SEQ ID NO: 43) and/or a variable heavy chain sequence comprising: QSVEESGGRLVTPGTPLTLTCTASGFSLSNV YIHWVRQAPGKGLEWIGYISDGDTARYATWAKGRFTISKTSSTTVNLKMTSLTTEDTATYFCARQ GFNIWGPGTLVTVSL (SEQ ID NO: 44). In some embodiments, the anti-ID3 antibody comprises a variable light chain sequence comprising: AVLTQTPSPVSAAVGGTVTSCQSSQSVYNNNWLSWFQQKSGQPP KLLIYETSKLESGVPSRFKGSGSGTQFTLTIIDVQCDDAATYYCLGGYWTTSDNNIFGGGTEVVVK (SEQ ID NO: 45) and/or a variable heavy chain sequence comprising: QSVEESGGRLVTPGTPLTLTCTASGFSLSSY YIHWVRQAPGKALEWIGYISDGGTTYYASWAKGRFTISKTSSTTVDLKMTSLTTEDTATYFCARQ GFNIWGPGTLVTVSL (SEQ ID NO: 46). In some embodiments, the anti-ID3 antibody comprises a variable light chain sequence comprising: AVLTQTPSPVSAAVGGTVSISCQSSQSVWNNNWLSWFQQKPGQPPKLL IYETSKLESGVPSRFKGSGSGTQFTLTISDVQCDDAATYYCLGGYWTTSDNNVFGGGTEVVVK (SEQ ID NO: 47) and/or a variable heavy chain sequence comprising: QSVEESGGRLVTPGTPLTLTCTASGFSLSNVYIHWVRQAPGKGLEWIGYISDGDTARYATWAKGRF TISKTSSTTVNLKMTSLTTEDTATYFCARQGFNIWGPGTLVTVSL (SEQ ID NO: 48). In some embodiments, the anti-ID3 antibody comprises a variable light chain sequence comprising: AVLTQTPSPVSAAVGGTV TISCQSSQSVYNNNWLSWFQQKSGQPPKWYETSKLESGVPSRFKGSGSGTQFTLTIIDVQCDDAA TYYCLGGYWSTSDNNIFGGGTEVVVK (SEQ ID NO: 49) and/or a variable heavy chain sequence comprising: QSVEESGGRLVTPGTPLTLTCTASGFSLSSYYIHWVRQAPGKALEWIGYISDGGTTYYASWAKGRF TISKTSSTTVDLKMTSLTTEDTATYFCARQGFNIWGPGTLVTVSL (SEQ ID NO: 50).

In a further aspect of the invention, an anti-USP1 antibody, an anti-UAF1 antibody and/or an anti-ID antibody according to any of the above embodiments is a monoclonal antibody, including a chimeric, humanized or human antibody. In one embodiment, an anti-USP1 antibody, an anti-UAF1 antibody and/or an anti-ID antibody is an antibody fragment, e.g., a Fv, Fab, Fab′, scFv, diabody, or F(ab′)₂ fragment. In another embodiment, the antibody is a full length antibody, e.g., an intact IgG1″ antibody or other antibody class or isotype as defined herein.

In a further aspect, an anti-USP1 antibody, an anti-UAF1 antibody and/or an anti-ID antibody according to any of the above embodiments may incorporate any of the features, singly or in combination, as described in Sections below:

1. Antibody Affinity

In certain embodiments, an antibody provided herein has a dissociation constant (Kd) of ≦1 μM. In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CMS chips at −10 response units (RU). Briefly, carboxymethylated dextran biosensor chips (CMS, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMab® technology; U.S. Pat. No. 7,041,870 describing K-M MOUSE® technology, and U.S. Patent Application Publication No. US 2007/0061900, describing VelociMouse® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for USP1 or an ID (e.g., ID1, ID2, or ID3) and the other is for any other antigen. In certain embodiments, bispecific antibodies may bind to two different epitopes of USP1 or an ID (e.g., ID1, ID2, or ID3). Bispecific antibodies may also be used to localize cytotoxic agents to cells which express USP1 and/or an ID (e.g., ID1, ID2, and/or ID3). Bispecific antibodies can be prepared as full length antibodies or antibody fragments.

Techniques for making multispecific antibodies include, but are not limited to, recombinant coexpression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to USP1 or an ID (e.g., ID1, ID2, or ID3) as well as another, different antigen (see, US 2008/0069820, for example).

7. Antibody Variants

a) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

b) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).) In certain embodiments, an antibody variant comprises an Fc region with one or more amino acid substitutions which improve ADCC, e.g., substitutions at positions 298, 333, and/or 334 of the Fc region (EU numbering of residues). In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. No. 5,648,260; U.S. Pat. No. 5,624,821; and WO 94/29351 concerning other examples of Fc region variants.

c) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antibodies may be generated as described, e.g., in U.S. Pat. No. 7,521,541.

### B. Immunoconjugates

Further provided herein are immunoconjugates comprising an anti-USP1 antibody and/or an anti-ID antibody (e.g., anti-ID1 antibody, anti-ID2 antibody, or anti-ID3 antibody) herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc⁹⁹ or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, STAB, SMCC, SMPB, SMPH, sulfo-EMCS, sulfo-GMBS, sulfo-KMUS, sulfo-MBS, sulfo-STAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### C. Binding Polypeptides

Binding polypeptides are polypeptides that bind, preferably specifically, to USP1, UAF1, and/or ID (e.g., ID1, ID2, and/or ID3) as described herein. Binding polypeptides may be chemically synthesized using known polypeptide synthesis methodology or may be prepared and purified using recombinant technology. Binding polypeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such binding polypeptides that are capable of binding, preferably specifically, to a target, USP1, UAF1, and/or ID (e.g., ID1, ID2, and/or ID3), as described herein. Binding polypeptides may be identified without undue experimentation using well known techniques. In this regard, it is noted that techniques for screening polypeptide libraries for binding polypeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

In this regard, bacteriophage (phage) display is one well known technique which allows one to screen large polypeptide libraries to identify member(s) of those libraries which are capable of specifically binding to a target polypeptide, USP1, UAF1, and/or ID (e.g., ID1, ID2, and/or ID3). Phage display is a technique by which variant polypeptides are displayed as fusion proteins to the coat protein on the surface of bacteriophage particles (Scott, J. K. and Smith, G. P. (1990) Science, 249: 386). The utility of phage display lies in the fact that large libraries of selectively randomized protein variants (or randomly cloned cDNAs) can be rapidly and efficiently sorted for those sequences that bind to a target molecule with high affinity. Display of peptide (Cwirla, S. E. et al. (1990) Proc. Natl. Acad. Sci. USA, 87:6378) or protein (Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363) libraries on phage have been used for screening millions of polypeptides or oligopeptides for ones with specific binding properties (Smith, G. P. (1991) Current Opin. Biotechnol., 2:668). Sorting phage libraries of random mutants requires a strategy for constructing and propagating a large number of variants, a procedure for affinity purification using the target receptor, and a means of evaluating the results of binding enrichments. U.S. Pat. Nos. 5,223,409, 5,403,484, 5,571,689, and 5,663,143.

Although most phage display methods have used filamentous phage, lambdoid phage display systems (WO 95/34683; U.S. Pat. No. 5,627,024), T4 phage display systems (Ren et al., Gene, 215: 439 (1998); Zhu et al., Cancer Research, 58(15): 3209-3214 (1998); Jiang et al., Infection & Immunity, 65(11): 4770-4777 (1997); Ren et al., Gene, 195(2):303-311 (1997); Ren, Protein Sci., 5: 1833 (1996); Efimov et al., Virus Genes, 10: 173 (1995)) and T7 phage display systems (Smith and Scott, Methods in Enzymology, 217: 228-257 (1993); U.S. Pat. No. 5,766,905) are also known.

Additional improvements enhance the ability of display systems to screen peptide libraries for binding to selected target molecules and to display functional proteins with the potential of screening these proteins for desired properties. Combinatorial reaction devices for phage display reactions have been developed (WO 98/14277) and phage display libraries have been used to analyze and control bimolecular interactions (WO 98/20169; WO 98/20159) and properties of constrained helical peptides (WO 98/20036). WO 97/35196 describes a method of isolating an affinity ligand in which a phage display library is contacted with one solution in which the ligand will bind to a target molecule and a second solution in which the affinity ligand will not bind to the target molecule, to selectively isolate binding ligands. WO 97/46251 describes a method of biopanning a random phage display library with an affinity purified antibody and then isolating binding phage, followed by a micropanning process using microplate wells to isolate high affinity binding phage. The use of Staphlylococcus aureus protein A as an affinity tag has also been reported (Li et al. (1998) Mol Biotech., 9:187). WO 97/47314 describes the use of substrate subtraction libraries to distinguish enzyme specificities using a combinatorial library which may be a phage display library. A method for selecting enzymes suitable for use in detergents using phage display is described in WO 97/09446. Additional methods of selecting specific binding proteins are described in U.S. Pat. Nos. 5,498,538, 5,432,018, and WO 98/15833.

Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

In some embodiments, the binding polypeptide is an USP1 antagonist. In some embodiments, the binding polypeptide is an UAF1 antagonist. In some embodiments, the binding polypeptide is an ID1 antagonist. In some embodiments, the binding polypeptide is an ID2 antagonist. In some embodiments, the binding polypeptide is an ID3 antagonist. In some embodiments, the binding polypeptide is capable of inhibiting more than one ID (e.g., two IDs, three IDs, or four IDs). In some embodiments, the binding polypeptide inhibits interaction of USP1 with UAF1. In some embodiments, the binding polypeptide blocks deubiquitination of ID. In some embodiment, the binding polypeptide inhibits interaction of ID with bHLH. In some embodiments, the binding polypeptide inhibits cleavage of USP1.

In some embodiments, the binding polypeptide is an ID antagonist and the ID antagonist is polypeptide which inhibits the transport of an ID protein to the cytoplasm. In some embodiments, the binding polypeptide is an ID antagonist and the ID antagonist is polypeptide which sequesters an ID protein in the cytoplasm. In some embodiments, the binding polypeptide is an ID antagonist and the ID antagonist is a protein comprising at least one LIM domain. The LIM domain is a cysteine-rich double zinc finger motif, which mediates protein-protein interactions. In some embodiments, the binding polypeptide is an ID antagonist and the ID antagonist is a protein comprising at least one LIM-PDZ protein. A “LIM-PDZ protein family” member, or “LIM-PDZ” protein, refers to a naturally occurring group of proteins (and homologues, mutants, variants thereof) that share a high degree of amino acid similarity in their PDZ and LIM protein domains (up to 70% sequence similarity). The family now contains seven proteins, each of which contains one N-terminal PDZ domain followed either by one C-terminal LIM domain (ALP subfamily; ALP, RIL, CLP-36/hClim1/Elfin, Mystique) or three C-terminal LIM domains (Enigma subfamily; Enigma/LMP-1, ENH, ZASP/Cypher1) (Xia et al., J. Cell Biol., 271: 15934-15941, 1997). In some embodiments, the binding polypeptide is an ID antagonist and the ID antagonist is an enigma homolog (ENH) protein or fragment thereof. See, e.g., US Patent Publication No. 2007/0041944, the contents of which are incorporated by reference in its entirety. In some embodiments, the binding polypeptide is an ID2 antagonist and the ID2 antagonist is an ENH protein thereof. In some embodiments, the ENH protein comprises the amino acid sequence (SEQ ID NO: 51)

| | |
|---|---|
| 1 | MSNYSVSLVG PAPWGFRLQG GKDFNMPLTI SSLKDGGKAA |
| | QANVRIGDVV LSIDGINAQG |
| 61 | MTHLEAQNKI KGCTGSLNMT LQRASAAPKP EPVPVQKGEP |
| | KEVVKPVPIT SPAVSKVTST |
| 121 | NNMAYNKAPR PFGSVSSPKV TSIPSPSSAF TPAHATTSSH |
| | ASPSPVAAVT PPLFAASGLH |
| 181 | ANANLSADQS PSALSAGKTA VNVPRQPTVT SVCSETSQEL |
| | AEGQRRGSQG DSKQQNGPPR |
| 241 | KHIVERYTEF YHVPTHSDAS KKRLIEDTED WRPRTGTTQS |
| | RSFRILAQIT GTEHLKESEA |
| 301 | DNTKKANNSQ EPSPQLASSV ASTRSMPESL DSPTSGRPGV |
| | TSLTTAAAFK PVGSTGVIKS |
| 361 | PSWQRPNQGV PSTGRISNSA TYSGSVAPAN SALGQTQPSD |
| | QDTLVQRAEH IPAGKRTPMC |
| 421 | AHCNQVIRGP FLVALGKSWH PEEFNCAHCK NTMAYIGFVE |
| | EKGALYCELC YEKFFAPECG |
| 481 | RCQRKILGEV INALKQTWHV SCFVCVACGK PIRNNVFHLE |
| | DGEPYCETDY YALFGTICHG |
| 541 | CEFPIEAGDM FLEALGYTWH DTCFVCSVCC ESLEGQTFFS |
| | KKDKPLCKKH AHSVNF |

In some embodiments, the ENH protein comprises the amino acid sequence (SEQ ID NO: 52):

| | |
|---|---|
| 1 | MSNYSVSLVG PAPWGFRLQG GKDFNMPLTI SSLKDGGKAA |
| | QANVRIGDVV LSIDGINAQG |
| 61 | MTHLEAQNKI KGCTGSLNMT LQRASAAPKP EPVPVQKPTV |
| | TSVCSETSQE LAEGQRRGSQ |
| 121 | GDSKQQNGPP RKHIVERYTE FYHVPTHSDA SKKRLIEDTE |
| | DWRPRTGTTQ SRSFRILAQI |
| 181 | TGTEHLKESE ADNTKKANNS QEPSPQLASS VASTRSMPES |
| | LDSPTSGRPG VTSLTTAAAF |
| 241 | KPVGSTGVIK SPSWQRPNQG VPSTGRISNS ATYSGSVAPA |
| | NSALGQTQPS DQDTLVQRAE |
| 301 | HIPAGKRTPM CAHCNQVIRG PFLVALGKSW HPEEFNCAHC |
| | KNTMAYIGFV EEKGALYCEL |
| 361 | CYEKFFAPEC GRCQRKILGE VINALKQTWH VSCFVCVACG |
| | KPIRNNVFHL EDGEPYCETD |
| 421 | YYALFGTICH GCEFPIEAGD MFLEALGYTW HDTCFVCSVC |
| | CESLEGQTFF SKKDKPLCKK |
| 481 | HAHSVNF |

In some embodiments, the binding polypeptide is an UAF1 antagonist and the UAF1 antagonist is polypeptide which binds USF1 WD40 repeat(s), e.g., WD40 repeats 2-4, WD40 repeat 2, WD40 repeat 3, WD40 repeat 4, WD40 repeat 8.

### D. Binding Small Molecules

Provided herein are binding small molecules for use as USP1 antagonists, UAF1 and/or ID antagonists (e.g., ID1 antagonist, ID2 antagonist, and/or ID3 antagonists).

Binding small molecules are preferably organic molecules other than binding polypeptides or antibodies as defined herein that bind, preferably specifically, to USP1, UAF1, and/or ID (e.g., ID1, ID2, and/or ID3) as described herein. Binding organic small molecules may be identified and chemically synthesized using known methodology (see, e.g., PCT Publication Nos. WO00/00823 and WO00/39585). Binding organic small molecules are usually less than about 2000 daltons in size, alternatively less than about 1500, 750, 500, 250 or 200 daltons in size, wherein such organic small molecules that are capable of binding, preferably specifically, to a polypeptide as described herein may be identified without undue experimentation using well known techniques. In this regard, it is noted that techniques for screening organic small molecule libraries for molecules that are capable of binding to a polypeptide target are well known in the art (see, e.g., PCT Publication Nos. WO00/00823 and WO00/39585). Binding organic small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

In some embodiments, the binding small molecule is an USP1 antagonist. In some embodiments, the binding small molecule is an UAF1 antagonist. In some embodiments, the binding small molecule is an ID1 antagonist. In some embodiments, the binding small molecule is an ID2 antagonist. In some embodiments, the binding small molecule is an ID3 antagonist. In some embodiments, the binding small molecule is capable of inhibiting more than one ID (e.g., two IDs, three IDs, or four IDs). In some embodiments, the binding small molecule inhibits interaction of USP1 with UAF1. In some embodiments, the binding small molecule blocks deubiquitination of ID. In some embodiment, the binding small molecule inhibits interaction of ID with bHLH. In some embodiments, the binding small molecule inhibits cleavage of USP1.

In some embodiments, the binding small molecule is an USP1 antagonist and the USP1 antagonist is ubiquitin aldehyde. In this case, the USP1 antagonist is thought to act by forming a tight complex with the USP1 enzyme, as described in Hershko et al. (Ubiquitin-aldehyde: a general inhibitor of ubiquitin-recycling processes. Proc Natl Acad Sci 1987 April; 84(7):1829-33), which is incorporated herein by reference. Ubiquitin aldehyde is available from, e.g., Enzo Life Sciences. In some embodiments, the binding small molecule is an USP1 antagonist and the USP1 antagonist is camptothecin. Camptothecin is thought to inhibit formation of USP1 and UAF1 complex. See, e.g., Mura et al. Mol Cell Biol (2011) 31:2462. In some embodiments, the binding small molecule is an USP1 antagonist and the USP1 antagonist NSC 632839 hydrochloride (3,5-Bis[(4-methylphenyl)methylene]-4-piperidone hydrochloride; CAS No. 157654-67-6)(Tocris).

In some embodiments, the binding small molecule is an ID antagonist and the ID antagonist is capable of inhibiting more than one ID (e.g., two IDs, three IDs, or four IDs). In some embodiments, the binding small molecule is an ID antagonist and the ID antagonist is capable of inhibiting ID1 and ID3. In some embodiments, the ID antagonist capable of inhibiting ID1 and ID3 is tetracycline. US Patent Publication No. 2003/0022871 describes the use of tetracycline as an antagonist of Id1 and Id3, the contents of which are incorporated by reference in its entirety. “Tetracycline” refers to a compound having an elemental formula of C₂₂H₂₄N₂O8 and nomenclature of [4S-(4I,5aI,5aI,6J,12aI)]-4-(Dimethylamino)-1,4,4a,5,5a,6-11,12a-octahydro-3,6,10,12,12a-peiztaiydroxy-6-methyl-1,11-dioxo-2-naphthacenecarboxamide. The structure of tetracycline is set forth below:

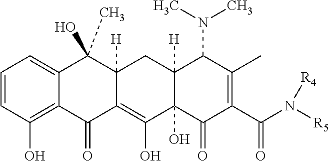

Alternatively, the compound comprises an analog or derivative of tetracycline. Numerous analogs and derivatives of tetracycline have applications in a method described herein. In a particular embodiment, an analog or derivative of tetracycline having applications herein has a general structure comprising:

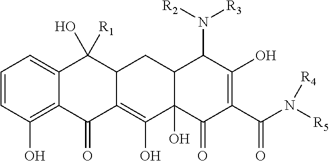

wherein R₁, R₂, R₃, R₄, and R₅ may be the same or different, and comprise H, lower alkyl (C₁-C₄), C₁-C₄ alkoxyl, cycloalkyl, aryl, or heterocyclic ring structures.

Other examples of analogs or derivatives of tetracycline having applications herein are set forth in U.S. Pat. Nos. 5,589,470; 5,064,821, 5,811,412; 4,089,900; 4,960,913; 4,066,694; 4,060,605; 3,911,111; and 3, 951,962, the contents of which are hereby incorporated by reference herein in their entireties.

### E. Antagonist Polynucleotides

Provided herein are polynucleotide antagonists. The polynucleotide may be an antisense nucleic acid and/or a ribozyme. The antisense nucleic acids comprise a sequence complementary to at least a portion of an RNA transcript of an USP1 gene, and UAF1 gene, and/or an ID gene (e.g., ID1, ID2 and/or ID3). However, absolute complementarity, although preferred, is not required. A sequence “complementary to at least a portion of an RNA,” referred to herein, means a sequence having sufficient complementarity to be able to hybridize with the RNA, forming a stable duplex; in the case of double stranded USP1, UAF1 and/or ID antisense nucleic acids, a single strand of the duplex DNA may thus be tested, or triplex formation may be assayed. The ability to hybridize will depend on both the degree of complementarity and the length of the antisense nucleic acid. Generally, the larger the hybridizing nucleic acid, the more base mismatches with an USP1, UAF1 and/or ID RNA it may contain and still form a stable duplex (or triplex as the case may be). One skilled in the art can ascertain a tolerable degree of mismatch by use of standard procedures to determine the melting point of the hybridized complex.

Polynucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., 1994, Nature 372:333-335. Thus, oligonucleotides complementary to either the 5′- or 3′-non-translated, non-coding regions of the USP1, UAF1 and/or ID gene, could be used in an antisense approach to inhibit translation of endogenous X mRNA. Polynucleotides complementary to the 5′ untranslated region of the mRNA should include the complement of the AUG start codon. Antisense polynucleotides complementary to mRNA coding regions are less efficient inhibitors of translation but could be used in accordance with the invention. Whether designed to hybridize to the 5′-, 3′- or coding region of USp1, UAF1 and/or ID mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

In one embodiment, the USP1, UAF1 and/or ID antisense nucleic acid of the invention is produced intracellularly by transcription from an exogenous sequence. For example, a vector or a portion thereof, is transcribed, producing an antisense nucleic acid (RNA) of the USP1, UAF1 and/or ID gene. Such a vector would contain a sequence encoding the USP1, UAF1 and/or ID antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others know in the art, used for replication and expression in vertebrate cells. Expression of the sequence encoding USP1, UAF1 and/or ID, or fragments thereof, can be by any promoter known in the art to act in vertebrate, preferably human cells. Such promoters can be inducible or constitutive. Such promoters include, but are not limited to, the SV40 early promoter region (Bernoist and Chambon, Nature 29:304-310 (1981), the promoter contained in the 3′ long terminal repeat of Rous sarcoma virus (Yamamoto et al., Cell 22:787-797 (1980), the herpes thymidine promoter (Wagner et al., Proc. Natl. Acad. Sci. U.S.A. 78:1441-1445 (1981), the regulatory sequences of the metallothionein gene (Brinster, et al., Nature 296:39-42 (1982)), etc.

Antagonist polynucleotides are disclosed and exemplified herein.

In some embodiments, the antagonist polynucleotide is an USP1 antagonist and the USP1 antagonist is 5′-TTGGCAAGTTATGAATTGATA-3′ (SEQ ID NO: 53) and/or 5′-TCGGCAATACTTGCTATCTTA-3′(SEQ ID NO: 54). In one embodiment, the antagonist polynucleotide is an USP1 antagonist and the USP1 antagonist is 5′-ACAGTTCGCTTCTACACAA-3′ (SEQ ID NO: 55). See, e.g., US Patent Publication No. 2010/0330599, the contents of which is hereby incorporated by reference herein in its entirety.

In some embodiments, the antagonist polynucleotide is an ID2 antagonist and the ID2 antagonist is 5′-gcggtgttcatgatttctt-3′ (SEQ ID NO: 56) and/or 5′-caaagcactgtgtgtgggctga-3′ (SEQ ID NO: 57). In some embodiments, the antagonist polynucleotide is an ID2 antagonist and the ID2 antagonist is disclosed in WO1997/005283WO2009/059201 and WO1997/005283, the contents of which are hereby incorporated by reference herein in their entireties.

In some embodiments, the antagonist polynucleotide is an ID1, ID2, ID3 and/or and ID4 antagonist and the ID1, ID2, ID3 and/or and ID4 antagonist is disclosed in WO2001/066116, the contents of which is hereby incorporated by reference in its entirety.

In some embodiments, the antagonist polynucleotide is an UAF1 antagonist and the UAF1 antagonist is 5′-CCGGTCGAGACTCTATCATAA-3′ (SEQ ID NO: 58) and/or 5′-CACAAGCAAGATCCATATATA-3′(SEQ ID NO: 59). In some embodiments, the antagonist polynucleotide is an UAF1 antagonist and the UAF1 antagonist is 5′-CAAGCAAGATCCATATATA-3′ (SEQ ID NO: 60).

### F. Antibody and Binding Polypeptide Variants

In certain embodiments, amino acid sequence variants of the antibodies and/or the binding polypeptides provided herein are contemplated. For example, it may be desirable to improve the binding affinity and/or other biological properties of the antibody. Amino acid sequence variants of an antibody and/or binding polypeptides may be prepared by introducing appropriate modifications into the nucleotide sequence encoding the antibody and/or binding polypeptide, or by peptide synthesis. Such modifications include, for example, deletions from, and/or insertions into and/or substitutions of residues within the amino acid sequences of the antibody and/or binding polypeptide. Any combination of deletion, insertion, and substitution can be made to arrive at the final construct, provided that the final construct possesses the desired characteristics, e.g., target-binding.

In certain embodiments, antibody variants and/or binding polypeptide variants having one or more amino acid substitutions are provided. Sites of interest for substitutional mutagenesis include the HVRs and FRs. Conservative substitutions are shown in Table 1 under the heading of “conservative substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of the antibody and/or the binding polypeptide that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### G. Antibody and Binding Polypeptide Derivatives

In certain embodiments, an antibody and/or binding polypeptide provided herein may be further modified to contain additional nonproteinaceous moieties that are known in the art and readily available. The moieties suitable for derivatization of the antibody and/or binding polypeptide include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody and/or binding polypeptide to be improved, whether the antibody derivative and/or binding polypeptide derivative will be used in a therapy under defined conditions, etc.

In another embodiment, conjugates of an antibody and/or binding polypeptide to nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### H. Recombinant Methods and Compositions

Antibodies and/or binding polypeptides may be produced using recombinant methods and compositions, e.g., as described in U.S. Pat. No. 4,816,567. In one embodiment, isolated nucleic acid encoding an anti-USP1 antibody, an anti-USP1 antibody or an anti-ID antibody (e.g., anti-ID1 antibody, anti-ID2 antibody, or anti-ID3 antibody). Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid encoding the antibody and/or binding polypeptide are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an antibody such as an anti-USP1 antibody, an anti-UAF1 antibody and/or an anti-ID antibody (e.g., anti-ID1 antibody, anti-ID2 antibody, or anti-ID3 antibody) and/or binding polypeptide is provided, wherein the method comprises culturing a host cell comprising a nucleic acid encoding the antibody and/or binding polypeptide, as provided above, under conditions suitable for expression of the antibody and/or binding polypeptide, and optionally recovering the antibody and/or polypeptide from the host cell (or host cell culture medium).

For recombinant production of an antibody such as an anti-USP1 antibody, an anti-UAF1 antibody and/or an anti-ID antibody (e.g., anti-ID1 antibody, anti-ID2 antibody, or anti-ID3 antibody) and/or binding polypeptide, nucleic acid encoding an antibody and/or binding polypeptide, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody and/or glycosylated binding polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

While the description relates primarily to production of antibodies and/or binding polypeptides by culturing cells transformed or transfected with a vector containing antibody- and binding polypeptide-encoding nucleic acid. It is, of course, contemplated that alternative methods, which are well known in the art, may be employed to prepare antibodies and/or binding polypeptides. For instance, the appropriate amino acid sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the antibody or binding polypeptide may be chemically synthesized separately and combined using chemical or enzymatic methods to produce the desired antibody or binding polypeptide.

Forms of antibody and binding polypeptide may be recovered from culture medium or from host cell lysates. If membrane-bound, it can be released from the membrane using a suitable detergent solution (e.g. Triton-X 100) or by enzymatic cleavage. Cells employed in expression of antibody and binding polypeptide can be disrupted by various physical or chemical means, such as freeze-thaw cycling, sonication, mechanical disruption, or cell lysing agents.

It may be desired to purify antibody and binding polypeptide from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the antibody and binding polypeptide. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular antibody or binding polypeptide produced.

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2 or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### III. Methods of Screening and/or Identifying USP1 Antagonists, UAF1 Antagonists and/or Id Antagonists with Desired Function

Techniques for generating antibodies, binding polypeptides, and/or small molecules have been described above. One may further select antibodies such as anti-USP1 antibodies, anti-UAF1 antibodies and/or an anti-ID antibody (e.g., anti-ID1 antibody, anti-ID2 antibody, or anti-ID3 antibody), binding polypeptides, and/or binding small molecules provided herein may be identified, screened for, or characterized for their physical/chemical properties and/or biological activities by various assays known in the art.

The growth inhibitory effects of an antibody, binding polypeptide or binding small molecules of the invention may be assessed by methods known in the art, e.g., using cells which express USP1, UAF1, and/or ID (e.g., ID1, ID2, and/or ID3) either endogenously or following transfection with the respective gene(s). For example, appropriate tumor cell lines, and USP1, UAF1, and/or ID (e.g., ID1, ID2, and/or ID3) polypeptide-transfected cells may be treated with a monoclonal antibody, binding polypeptide or other small molecule of the invention at various concentrations for a few days (e.g., 2-7) days and stained with crystal violet or MTT or analyzed by some other colorimetric assay. Another method of measuring proliferation would be by comparing ³H-thymidine uptake by the cells treated in the presence or absence an antibody, binding polypeptide or binding small molecule of the invention. After treatment, the cells are harvested and the amount of radioactivity incorporated into the DNA quantitated in a scintillation counter. Appropriate positive controls include treatment of a selected cell line with a growth inhibitory antibody known to inhibit growth of that cell line. Growth inhibition of tumor cells in vivo can be determined in various ways known in the art. The tumor cell may be one that overexpresses an USP1, UAF1, and/or ID (e.g., ID1, ID2, and/or ID3) polypeptide. The antibody, binding polypeptide, and/or binding small molecule will inhibit cell proliferation of USP1, UAF1, and/or ID (e.g., ID1, ID2, and/or ID3)-expressing tumor cell in vitro or in vivo by about 25-100% compared to the untreated tumor cell, more preferably, by about 30-100%, and even more preferably by about 50-100% or about 70-100%, in one embodiment, at an antibody concentration of about 0.5 to 30 μg/ml. Growth inhibition can be measured at an antibody concentration of about 0.5 to about 30 μg/ml or about 0.5 nM to about 200 nM in cell culture, where the growth inhibition is determined 1-10 days after exposure of the tumor cells to the antibody. The antibody is growth inhibitory in vivo if administration of the antibody at about 1 μg/kg to about 100 mg/kg body weight results in reduction in tumor size or reduction of tumor cell proliferation within about 5 days to 3 months from the first administration of the antibody, preferably within about 5 to 30 days.

To select for an antibody, binding polypeptide, and/or binding small molecule which inhibits deubiquitination, deubiquitinase activity or USP1 and/or UAF1 may be measured according to methods disclosed in US2010/0330599 and US2007/0061907, the contents of which are hereby incorporated by reference in their entireties.

To select for an antibody, binding polypeptide, and/or binding small molecule which induces cell death, loss of membrane integrity as indicated by, e.g., propidium iodide (PI), trypan blue or 7AAD uptake may be assessed relative to control. A PI uptake assay can be performed in the absence of complement and immune effector cells. USP1, UAF1, and/or ID (e.g., ID1, ID2, and/or ID3) polypeptide-expressing tumor cells are incubated with medium alone or medium containing the appropriate antibody (e.g, at about 10 μg/ml), binding polypeptide or binding small molecule. The cells are incubated for a 3-day time period. Following each treatment, cells are washed and aliquoted into 35 mm strainer-capped 12×75 tubes (1 ml per tube, 3 tubes per treatment group) for removal of cell clumps. Tubes then receive PI (10 μg/ml). Samples may be analyzed using a FACSCAN® flow cytometer and FACSCONVERT® CellQuest software (Becton Dickinson). Those antibodies, binding polypeptides or binding small molecules that induce statistically significant levels of cell death as determined by PI uptake may be selected as cell death-inducing antibodies, binding polypeptides or binding small molecules.

To screen for antibodies, binding polypeptides, and/or binding small molecules which bind to an epitope on a polypeptide bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a test antibody, binding polypeptide or binding small molecule binds the same site or epitope as a known antibody. Alternatively, or additionally, epitope mapping can be performed by methods known in the art. For example, the antibody sequence can be mutagenized such as by alanine scanning, to identify contact residues. The mutant antibody is initially tested for binding with polyclonal antibody to ensure proper folding. In a different method, peptides corresponding to different regions of a polypeptide can be used in competition assays with the test antibodies or with a test antibody and an antibody with a characterized or known epitope.

Provided herein are methods of screening for and/or identifying an USP1 antagonist, UAF1 antagonist, and/or an ID antagonist which promotes a change in cell fate said method comprising: comparing (i) a reference cell fate, wherein the reference cell fate is the cell fate of a reference cell with (ii) a candidate cell fate, wherein the candidate cell fate is the cell fate of the reference cell in the presence of an USP1 candidate antagonist, UAF1 candidate antagonist, and/or an ID candidate antagonist, wherein the USP1 candidate antagonist binds USP1, wherein the UAF1 candidate antagonist binds UAF1, and/or the ID candidate antagonist binds ID, whereby a difference in cell fate between the reference cell fate and the candidate cell fate identifies the USP1 candidate antagonist and/or the ID candidate antagonist as promoting a change in cell fate.

Provided herein are also methods of screening for and/or identifying an USP1 antagonist, UAF1 antagonist, and/or an ID antagonist which induces cell cycle arrest said method comprising: comparing (i) contacting a reference cell in the presence of an USP1 candidate antagonist, UAF1 candidate antagonist, and/or an ID candidate antagonist, wherein the USP1 candidate antagonist binds USP1, wherein the UAF1 candidate antagonist binds UAF1, and/or the ID candidate antagonist binds ID, whereby cell cycle arrest identifies the USP1 candidate antagonist and/or the ID candidate antagonist as inducing cell cycle arrest.

In some embodiments of any of the methods of screening, the USP1 candidate antagonist, UAF1 candidate antagonist, and/or the ID candidate antagonist is USP1 candidate antagonist. In some embodiments of any of the methods of screening, the USP1 candidate antagonist, UAF1 candidate antagonist, and/or the ID candidate antagonist is ID candidate antagonist. In some embodiments, the ID candidate antagonist is an ID1 candidate antagonist, an ID2 candidate antagonist, and/or an ID3 candidate antagonist. In some embodiments of any of the methods of screening, the USP1 candidate antagonist, UAF1 antagonist, and/or the ID candidate antagonist is UAF1 candidate antagonist.

In some embodiments of any of the methods of screening, the reference cell fate is a stem cell fate. In some embodiments, the stem cell fate is a mesenchymal stem cell fate. In some embodiments of any of the methods of screening, the candidate cell fate is an osteoblast cell fate, chondrocyte cell fate, or adipocyte cell fate. In some embodiments, the candidate cell fate is an osteoblast cell fate.

In some embodiments of any of the methods of screening, the USP1 candidate antagonist, UAF1 candidate antagonist, and/or the ID candidate antagonist is an antibody, binding polypeptide, binding small molecule, or polynucleotide.

1. Binding Assays and Other Assays

In one aspect, an antibody of the invention is tested for its antigen binding activity, e.g., by known methods such as ELISA, Western blot, etc.

### J. Methods and Compositions for Diagnostics and Detection

In certain embodiments, any of the anti-USP1 antibodies, anti-UAF1 antibodies, and/or anti-ID antibodies (e.g., ID1, ID2, and/or ID3) provided herein is useful for detecting the presence of USP1, UAF1, and/or ID (e.g., ID1, ID2, and/or ID3) in a biological sample. In certain embodiments, any of the anti-USP1 binding polypeptides, anti-UAF1 binding polypeptides, and/or anti-ID binding polypeptides (e.g., ID1, ID2, and/or ID3) provided herein is useful for detecting the presence of USP1, UAF1, and/or ID (e.g., ID1, ID2, and/or ID3) in a biological sample. The term “detecting” as used herein encompasses quantitative or qualitative detection. In certain embodiments, a biological sample comprises a cell or tissue, such as bone.

In one embodiment, anti-USP1 antibodies, anti-UAF1 antibodies, and/or anti-ID antibodies (e.g., ID1, ID2, and/or ID3) for use in a method of diagnosis or detection are provided. In a further aspect, a method of detecting the presence of USP1, UAF1, and/or ID (e.g., ID1, ID2, and/or ID3) in a biological sample is provided. In a further aspect, provided are methods of identifying an individual as suitable for treatment with an USP1, UAF1 and/or ID antagonist comprising determining expression (e.g., expression levels) of one or more genes selected from the group consisting of USP1, UAF1 and/or ID (e.g, ID1, ID3 and/or ID3). In a further aspect, provided are methods of identifying an individual as suitable for treatment with an USP1, UAF1 and/or ID antagonist comprising determining expression (e.g., expression levels) of one or more genes selected from the group consisting of CD90, CD105, CD106, USP1, UAF1, and ID (e.g., ID1, ID2, or ID3) (e.g., compared to a reference value and/or to an internal reference (e.g., CD144)). In some embodiments, the individual is selected for treatment based on elevated expression levels of one or more genes selected from the group consisting of CD90, CD105, CD106, USP1, UAF1, and ID (e.g., ID1, ID2, or ID3) (e.g., compared to a reference value and/or to an internal reference (e.g., CD144)). In some embodiments, the individual is not selected for the treatment based upon low expression levels of one or more genes selected from the group consisting of CD90, CD105, CD106, USP1, UAF1, and ID (e.g., ID1, ID2, or ID3) (e.g., compared to a reference value and/or an internal reference (e.g., CD144)). In some embodiments, the individual is selected for the treatment based upon low expression levels of one or more genes selected from the group consisting of p21, RUNX2, OSTERIX, SPARC/OSTEONECTIN, SPP1/OSTEOPONTIN, BGLAP/OSTEOCALCIN, and alkaline phosphatase (ALP) (e.g., compared to a reference value and/or an internal reference (e.g., CD144)). In some embodiments, the individual is not selected for the treatment based upon elevated expression levels of one or more genes selected from the group consisting of p21, RUNX2, OSTERIX, SPARC/OSTEONECTIN, SPP1/OSTEOPONTIN, BGLAP/OSTEOCALCIN, and alkaline phosphatase (ALP) (e.g., compared to a reference value and/or an internal reference (e.g., CD144)).

In certain embodiments, expression is protein expression. In certain embodiments, expression is polynucleotide expression. In certain embodiments, the polynucleotide is DNA. In certain embodiments, the polynucleotide is RNA.

Various methods for determining expression of mRNA, protein, or gene amplification include, but are not limited to, gene expression profiling, polymerase chain reaction (PCR) including quantitative real time PCR (qRT-PCR), RNA-Seq, FISH, microarray analysis, serial analysis of gene expression (SAGE), MassARRAY, proteomics, immunohistochemistry (IHC), etc. In some embodiments, protein expression is quantified. Such protein analysis may be performed using IHC, e.g., on patient tumor samples.

In one aspect, level of biomarker is determined using a method comprising: (a) performing gene expression profiling, PCR (such as rtPCR), RNA-seq, microarray analysis, SAGE, MassARRAY technique, or FISH on a sample (such as a patient cancer sample); and b) determining expression of a biomarker in the sample. In one aspect, level of biomarker is determined using a method comprising: (a) performing IHC analysis of a sample (such as a patient cancer sample) with an antibody; and b) determining expression of a biomarker in the sample. In some embodiments, IHC staining intensity is determined relative to a reference value.

In certain embodiments, the method comprises contacting the biological sample with anti-USP1 antibodies, anti-UAF1 antibodies, and/or anti-ID antibodies (e.g., ID1, ID2, and/or ID3) as described herein under conditions permissive for binding of the anti-USP1 antibody to USP1, UAF1, and/or ID (e.g., ID1, ID2, and/or ID3), and detecting whether a complex is formed between the anti-USP1 antibodies, anti-UAF1 antibodies, and/or anti-ID antibodies (e.g., ID1, ID2, and/or ID3) and USP1, UAF1, and/or ID (e.g., ID1, ID2, and/or ID3). Such method may be an in vitro or in vivo method. In one embodiment, an anti-USP1 antibody is used to select subjects eligible for therapy with anti-USP1 antibodies, anti-UAF1 antibodies, and/or anti-ID antibodies (e.g., ID1, ID2, and/or ID3), e.g. where USP1, UAF1, and/or ID (e.g., ID1, ID2, and/or ID3) is a biomarker for selection of patients.

In certain embodiments, labeled anti-USP1 antibodies, anti-UAF1 antibodies, and/or anti-ID antibodies (e.g., ID1, ID2, and/or ID3) are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### K. Pharmaceutical Formulations

Pharmaceutical formulations of an USP1 antagonist, UAF1 antagonists and/or an ID antagonist (e.g., ID1 antagonist, ID2 antagonist, or ID3 antagonist) as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. In some embodiments, the USP1 antagonist and/or an ID antagonist (e.g., ID1 antagonist, ID2 antagonist, or ID3 antagonist) is a binding small molecule, an antibody, binding polypeptide, or polynucleotide. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### L. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an USP1 antagonist, an UAF1 antagonist and/or an ID antagonist (e.g., ID1 antagonist, ID2 antagonist, or ID3 antagonist); and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to an anti-USP1 antibody, anti-UAF1 antibody, and/or an anti-ID antibody (e.g., anti-ID1 antibody, anti-ID2 antibody, or anti-ID3 antibody).

### EXAMPLES

The following are examples of methods and compositions of the invention. It is understood that various other embodiments may be practiced, given the general description provided above.

### Materials and Methods for the Examples

Cell Lines and Culture Conditions

The human cell lines 143B, 293T, HOS, MG-63, SAOS-2, SJSA, and U2-OS (ATCC) were maintained in DMEM with 10% FBS (Sigma), 10 units/ml penicillin, and 10 μg/ml streptomycin (Gibco). Primary human osteoblasts (PromoCell) were expanded in primary osteoblast medium (PromoCell) and subcultured in DMEM supplemented as above. Primary human mesenchymal stem cells derived from normal bone marrow (Lonza) were subcultured in mesenchymal stem cell growth medium (Lonza). For osteogenic differentiation studies, hMSC were cultured in osteogenic differentiation medium (Lonza) supplemented with 100 ng/mL BMP-9 (R&D Systems). Primary human osteosarcomas were obtained from CytoMix, LLC, and from the Cooperative Human Tissue Network. Primary osteoblasts were used at passage 2-3, and were validated by expression of alkaline phosphatase, alazarin red reactivity, and osteoblast-specific transcripts Osterix and Osteonectin as assessed by RT-PCR. Murine NIH-3T3 (ATCC) were cultured in supplemented DMEM. Wild-type and USP1^{-/-} DT-40 cells were a kind gift from K. Patel and were cultured in RPMI with 7% FBS and 3% chicken serum (Gibco). MG-132 (Calbiochem) was used at 10 μM. Cycloheximide (Sigma) was used at 25 μg/ml.

### Expression Vectors

cDNAs for human deubiquitinases, including USP1 and mutant USP1 C905, were synthesized (Blue Heron Biotechnology) and cloned into pRK2001 with or without an in-frame C-terminal Flag epitope. shRNA-resistant USP1 was generated via codon-preserving site-directed mutagenesis. ID1, ID2, and ID3 were amplified from Jurkat-derived cDNAs and cloned in-frame with a C-terminal Flag epitope into pRK2001. WDR48 was amplified from an expression vector (Origene) and subcloned into pRK2001. The plasmid encoding HA-ubiquitin has been described (Wertz, et al., Nature 430, 694, 2004). pMACS, a truncated murine MHC class I H-2K^{k}-expression vector, was obtained from Miltenyi Biotec. For viral expression studies, ID2 and USP1 variants were cloned into the retroviral vector pQCXIP (Clontech) or the lentiviral vector pHUSH.Lenti.Puro (David Davis, Genentech, BMC Biotechnol. 2007 Sep. 26; 7:61).

shRNA vectors in the pRS expression vector targeting USP1 (A-TI333874 (SEQ ID NO:6) 5′-TGGTGGACTTTCCAAGATCAACACTCCTT-3′) or (B-TI333876 (SEQ ID NO:7) 5′-CAAGGAATCCAGTGACCAAACAGGCATTA-3′), ID1 (TI315979 (SEQ ID NO:8) 5′-GAGATTCTCCAGCACGTCATCGACTACAT-3′), ID2 (TI349048 (SEQ ID NO:9) 5′-CCTTCTGAGTTAATGTCAAATGACAGCAA-3′), ID3 (TI375157 (SEQ ID NO:10) 5′-TGGTCTCCTTGGAGAAAGGTTCTGTTGCC-3′) or a non-targeting control sequence (pRS30003 (SEQ ID NO:11) 5′-TGACCACCCTGACCTACGGCGTGCAGTGC-3′) were obtained from Origene. Unless otherwise indicated, shUSP1-B was used in USP1 knockdown experiments.

Doxycycline-inducible shRNA constructs in the pTRIPZ expression vector targeting human USP1 (5′-AGGCAATACTTGCTATCTTAAT-3′ (SEQ ID NO:12)), murine ID1 (5′-CGCAGCACGTCATCGACTACAT-3′(SEQ ID NO:13)), murine ID2 (5′-CGCAAAGTACTCTGTGGCTAAA-3′ (SEQ ID NO:14))(5′-CGCAGCACGTCATCGATTACAT-3′ (SEQ ID NO:15)), (5′-CTGACTGCTACTCCAAGCTCAA-3′ (SEQ ID NO:16)), murine ID3 (5′-CGCCCTGATTATGAACTCTATA-3′ (SEQ ID NO:17)), (5′-ACCTGATTATGAACTCTATAAT-3′ (SEQ ID NO:18)), (5′-CGCCCTCTTCACTTACCCTGAA-3′ (SEQ ID NO:19)) or a non-targeting control were obtained from Open Biosystems.

Transfection, Cell Sorting, RNA, and Protein Extraction

U2-OS, HOS, SJSA, SAOS, or MG-63 cells were grown to 10-25% confluence and transfected with the plasmids indicated in combination with the marker plasmid pMACS using FuGENE 6 (Sigma).

Transfected cells were sorted with MACS-select H-2K^{k} microbeads (Miltenyi Biotec). RNA was extracted from cultured or sorted transfected cells with Qiagen RNeasy Mini kits. Protein was extracted from sorted or cultured cells via lysis in NP-40 buffer (1% NP-40, 120 mM NaCl, 50 mM Tris, pH=7.4, 1 mM EDTA) supplemented with protease inhibitor cocktail I and phosphatase inhibitor cocktails 1 and 2 (Calbiochem). Lysates were clarified by centrifugation at 15,000×G for 10 minutes prior to analysis. Protein content was normalized by BCA protein assay (Thermo Scientific). For dual shRNA/siRNA experiments, cells were transfected with DNA expression vectors via Fugene and subsequently transfected with control siRNA (sense-5′-AAUUCUCCGAACGUGUCACGU-3′ (SEQ ID NO:20)) or siRNA targeting p21 (5′-CGATGGAACTTCGACTTTGTT-3′ (SEQ ID NO:21)) via nucleofection with program X-001 in nucleofection solution V (Lonza). DT-40 cells were transfected by nucleofection with program B-023 in nucleofection solution T (Lonza).

Antibodies, Western Blotting, and Immunoprecipitation

Rat monoclonal antibodies were raised against the C-terminal 100 amino-acids of human USP1 or WDR48 to produce the monoclonal USP1 antibody 5E10 and WDR48 antibody 9F10. Antibodies recognizing ID1, ID2, ID3, E47, and p53 (Santa Cruz Biotechnology), GAPDH (Assay Designs), Flag, HA, tubulin, and actin (Sigma), p21^{wAF1/CIP1} (Cell Signaling), E-cadherin and N-cadherin (BD Transduction Labs), and fibronectin (Calbiochem) were obtained from commercial sources. Immunoprecipitations were performed in the presence of 10 μM MG-132 with the indicated antibodies and protein A/G agarose beads (Pierce). Protein extracts were separated on Bis-Tris gels (Invitrogen) and transferred to 0.2 μM nitrocellulose membranes (Invitrogen) for immunoblot analysis.

RNA Analysis

RNA was extracted using Qiagen RNEasy RNA isolation kits. DNA oligonucleotide primers targeting USP1 (5′: 5′-GCCACTCAGCCAAGGCGACTG-3′ (SEQ ID NO:22); 3′: 5′-CAGAATGCCTCATACTGTCCATCTCTATGC-3′ (SEQ ID NO:23)), ID1 (5′: 5′-GAGCTGGTGCCCACCCTGC-3′ (SEQ ID NO:24); 3′: 5′-GATCGTCCGCAGGAACGCAT-3′ (SEQ ID NO:25)), ID2 (5′: 5′-CAAGAAGGTGAGCAAGATGGAAATCCT-3′(SEQ ID NO:26); 3′: 5′-ACAGTGCTTTGCTGTCATTTGACATTAACTC-3′ (SEQ ID NO:27)), ID3 (5′: 5′-GAGCCGCTGAGCTTGCTGGA-3′ (SEQ ID NO:28); 3′: 5′-ATGACAAGTTCCGGAGTGAGCTCG-3′ (SEQ ID NO:29)), p21 (5′: 5′-CTTGGCCTGCCCAAGCTCTACCTTCCCACG-3′ (SEQ ID NO:30); 3′: 5′-GGGCTTCCTCTTGGAGAAGATCAGCCGGCG-3′ (SEQ ID NO:31)), Runx2 (5′: 5′-ATGGGACTGTGGTTACTGTCATGGCGGG-3′ (SEQ ID NO:32); 3′: 5′-CTGGGTTCCCGAGGTCCATCTACTGTAACTTTAATTGC-3′ (SEQ ID NO:33)), Osterix (5′: 5′-CTCTCCATCTGCCTGGCTCCTTGGGAC-3′ (SEQ ID NO:34); 3′: 5′-CCTCAGGCTATGCTAATGATTACCCTCCCTTTTCCC-3′ (SEQ ID NO:35)), Osteonectin (5′: 5′-GCACCATGAGGGCCTGGATCTTCTTTCTCC-3′ (SEQ ID NO:36); 3′: 5′-GGTTCTGGCAGGGATTTTCCGCCACC-3′ (SEQ ID NO:37)) or β-actin (5′: 5′-GTCGACAACGGCTCCGGC-3′ (SEQ ID NO:38); 3′: 5′-GGTGTGGTGCCAGATTTTCT-3′ (SEQ ID NO:39)) were used to amplify mRNA from each respective gene using the QuantiTect SYBR Green RT-PCR system (Qiagen) and thermocycled with an ABI 7500 Real Time PCR System (Applied Biosystems). Data were analyzed with Sequence Detection Software v1.4 (Applied Biosystems). β-actin mRNA levels were used to normalize USP1 and ID mRNA levels to correct for loading and sample error.

Primary tumor RNA data was obtained from GeneLogic microarray analysis (Ocimum Biosolutions) of RNA expression levels in the indicated human bone samples using expression probes 208937_s_at (ID1), 213931_at (ID2), 207826_s_at (ID3), 202412_s_at (USP1), 202284_s_at (p21), 219534_x_at (p57), 236313_at (p15), and 207039_at (p16). Samples were hybridized to HGU133P Affymetrix chips.

Immunohistochemistry

Formalin-fixed, paraffin-embedded tissue sections were slide mounted, deparaffinized, and rehydrated with dH20. To recover antigen, samples were incubated in Target Retrieval Solution (Dako) at 99° C. for 20 minutes and cooled to 74° C. for 20 minutes. Endogenous peroxidase, avidin, biotin, and immunoglobulin were quenched by incubation in Avidin/Biotin Blocking Kit buffer (Vector Labs), followed by incubation in 3% BSA for 30 minutes at RT. Following quenching, samples were incubated in primary antibody for 60 minutes at RT, rinsed twice in Dako wash buffer, and incubated in Vectastain Kit (Vector Labs) buffer for 30 minutes. Staining was visualized by incubation in Peroxidase Substrate Buffer (Pierce). Samples were counterstained with Mayer's Hematoxylin and mounted with coverslips prior to imaging. RT=room temperature.

Flow Cytometry

Cell cycle analysis was performed on transfected cells by staining with FITC-conjugated H-2K^{k} antibodies (Miltenyi Biotec), followed by 70% ethanol fixation and DNA labeling with propidium iodide (Sigma) with RNAse A (Sigma) (Krishan, et al., J. Cell Biol. 66:188, 1975). DNA content of FITC+ cells was assessed using a FACSCalibur flow cytometer (BD Biosciences). Data were analyzed with FlowJo v8.7.3 software (Tree Star, Inc.). Cell cycle percentiles were quantified with the FlowJo cell cycle platform.

hMSC marker expression was assessed on osteosarcoma cell lines and hMSC by staining with PE-conjugated antibodies specific to CD90 (Chemicon), CD105 (R&D Systems), CD106 (SouthernBiotech), and CD144 (eBioscience), or an isotype control (R&D Systems). Geometric mean expression of markers was quantitated using a FACSCalibur flow cytometer.

Immunofluorescence Assays

U2-OS cells stably transduced with vectors as described were grown to confluence on chamber slides and treated with 3 μg/ml doxycycline (Clontech) for 14 days, fixed in 1% PFA in PBS, and probed with E-cadherin-FITC, N-cadherin (BD transduction laboratories), or fibronectin (Calbiochem). Unlinked antibodies were detected with goat-anti-mouse FITC (Southern Biotech Associates). Coverslips were mounted with ProLong Gold mounting agent (Invitrogen).

In Vivo Deubiquitination Assays

293T cells were transfected as described and treated with 10 μM MG-132 for 30 minutes prior to lysis in NP-40 buffer supplemented with 10 μM MG-132 and 10 mM N-ethylmaleimide (Sigma). Clarified lysates were dissociated with 1% SDS and boiled at 95° C. for 5 minutes, then diluted 1:20 in lysis buffer prior to immunoprecipitation with M2-agarose anti-Flag beads (Sigma). Ubiquitin levels were assessed by immunoblot with HA antibodies.

In Vitro Deubiquitination Assays

293T cells were transfected with USP1-Flag, USP1-C90S-Flag or ID2-Flag and HA-ubiquitin in separate batches. Ubiquitinated ID2-Flag was immunoprecipitated as described above from SDS-boiled lysates. USP1 and USP1 C90S were immunoprecipitated with Flag M2-agarose beads following lysis in NP-40 lysis buffer. All samples were eluted from beads with 500 μg/ml 3×Flag peptide (Sigma). Samples were recombined in deubiquitination buffer (20 mM HEPES, 20 mM NaCl, 100 μg/ml BSA, 500 μM EDTA, 1 mM DTT, pH=8.3) and incubated for the indicated times at room temperature. Ubiquitin levels were assessed by immunoblot with HA antibodies.

Osteosarcoma Differentiation Assay

U2-OS, HOS, or SAOS cells were transfected three times with pRS shUSP1 or shCTL as follows: Cells were initially transfected with shUSP1 or shCTL, cultured for 2 days, and selected with puromycin for 3 days. Cells were retransfected with pMACS and shUSP1 or shCTL, cultured for 2 days, sorted by anti-H-2K^{k} bead sort (Miltenyi), cultured for 1 day, and serially re-transfected with shUSP1 or shCTL. Cells were cultured for an additional 3 days and osteoblast and hMSC markers were assessed by flow cytometry, real-time RT-PCR, and ALP assay. 143B cells were transduced with pTRIPZ-based inducible USP1 or control shRNA vectors and puromycin-resistant cells were subcultured.

p-Nitrophenol-Phosphate Cleavage Assay

Cell lysates were produced with NP-40 buffer with proteasome inhibitors, but without phosphatase inhibitors and normalized for protein content. Lysates were added to p-nitrophenyl phosphatase substrate system solution (Sigma) in clear-bottomed 96-well plates and incubated at room temperature for 0.5-20 hours. A dilution series of known quantities of 4-nitrophenol (Sigma) was used as a reference. Activity was quantitated by OD absorbance measurement at 405 nm with a SpectraMax 190 spectrophotometer (Molecular Devices), and analyzed with SoftMax Pro v5.3 software (MDS Analytical Technologies). Data were normalized for protein input and reaction time.

Alizarin Red Staining

Differentiated hMSC plated on 8-well chamber slides were fixed with ice-cold 70% ethanol for 30 minutes, washed with deionized water, and incubated in 0.2% alizarin red stain (Ricca Chemical), pH 6.4, for 30 minutes. Following staining, cells were rinsed twice in deionized water and images were acquired.

3T3 Transformation Assay

Murine 3T3 fibroblasts were transduced with USP1-Flag, USP1-C90S-Flag, ID2-Flag, or an empty control expression vector and following two days of expression, plated in DMEM with FBS and penicillin/streptomycin with 0.5% low-melting agar on 1% agar beds. Cells were incubated for 21 days and colonies of 8 or more cells were scored by visual inspection. Transformed colonies from USP1-transformed samples were recovered from agar, passaged, and reseeded in soft agar to confirm transformation. Strong colony growth was observed in all passaged samples (data not shown).

For ID1, ID2, ID3 knockdown studies, USP1-transduced 3T3 cells were transduced with pTRIPZ-based ID1-, ID2,- and ID3-inducible shRNA expression vectors, or control shRNA vector and treated with 3 μg/ml doxycycline (Clontech) for 72 hours prior to agar embedding. 3 μg/ml doxycycline was included in both 1% and 0.5% agar.

In Vivo Studies

8-week-old female NCr nude mice (Taconic Laboratories, Hudson, N.Y.) or C.B-17 SCID.bg mice (Charles Rivers Laboratories, Hollister, Calif.) were injected subcutaneously in the right hind flank with 1×10⁶ murine 3T3 fibroblast cells (transduced with USP1, USP1-C905, ID-2, or vector control) in a volume of 100 μl of HBSS. Mice were monitored for tumor establishment and growth as well as body weight changes. When mice in a given group achieved a mean tumor volume of 2000 mm³ and/or reached 40 days post-inoculation, mice were euthanized and dissected to confirm the presence or absence of tumor formation.

8-week-old female NCr nude mice were injected subcutaneously in the right hind flank with 2.5×10⁶ 143 B shUSP1 cells in a volume of 100 μl of HBSS+matrigel. Doxycycline-treated mice were fed 1 mg/mL solutions of doxycycline in 5% sucrose water. Mice were monitored for tumor establishment and growth as well as body weight changes. When mice in a given group achieved a mean tumor volume of 2000 mm³ and/or reached 78 days post-inoculation, mice were euthanized and dissected to confirm the presence or absence of tumor formation.

Generation of USP1^{-/-} Mice

USP1 gene-targed C57BL/6 murine ES cells were obtained from the Knockout Mouse Project (KOMP) Repository (Davis, Calif.). The conditional allele was deleted in ES cells by electroporation with Cre recombinase prior to blastocyst injection.

Micro-Computed Tomography

d12.5 mouse pups, embryos 18.5 days post conception, or femurs dissected thereof were imaged with a μCT 40 (SCANCO Medical, Basserdorf, Switzerland) x-ray micro-computed tomography system with the following parameters: x-ray tube energy level=70 kV for femurs or 45 kV for whole mice, or x-ray tube energy level=45 kV, current=177 μA, integration time=300 msec, 1000 projections for femurs. Axial images were obtained at an isotropic resolution of 12 μm for femur analyses or 30 μm for fetuses/pups. A hydroxyapatite (HA) phantom was used to calibrate x-ray absorption to bone mineral density (BMD). Micro-computed tomography scans were analyzed with Analyze (AnalyzeDirect Inc., Lenexa, Kans., USA). Maximum-intensity projections and 3D surface renderings in the sagittal plane were created for each sample. Based on scan settings, a threshold followed by an erosion-dilation was applied to segment the mineralized skeleton from soft tissue.

In Situ TRAP Staining of Osteoclasts

Slide-mounted 5 μm sections from formalin fixed, paraffin embedded P12 murine femurs were prepared and subjected to TRAP staining (SIGMA) with the 387A Acid phosphatase, leukocyte (TRAP) kit according to the manufacturer's instructions. For enumeration studies, TRAP-positive osteoclasts were counted in ten fields.

Deoxypyridinoline and Creatinine Quantification

Amniotic fluid was collected from E18.5 mice, and deoxypyridinoline was detected by ELISA (TSZ ELISA) and creatinine was detected by colorimetric chemical assay (R&D Systems) as per manufacture's protocol.

### USP1 Deubiquitinates and Stabilizes ID Proteins

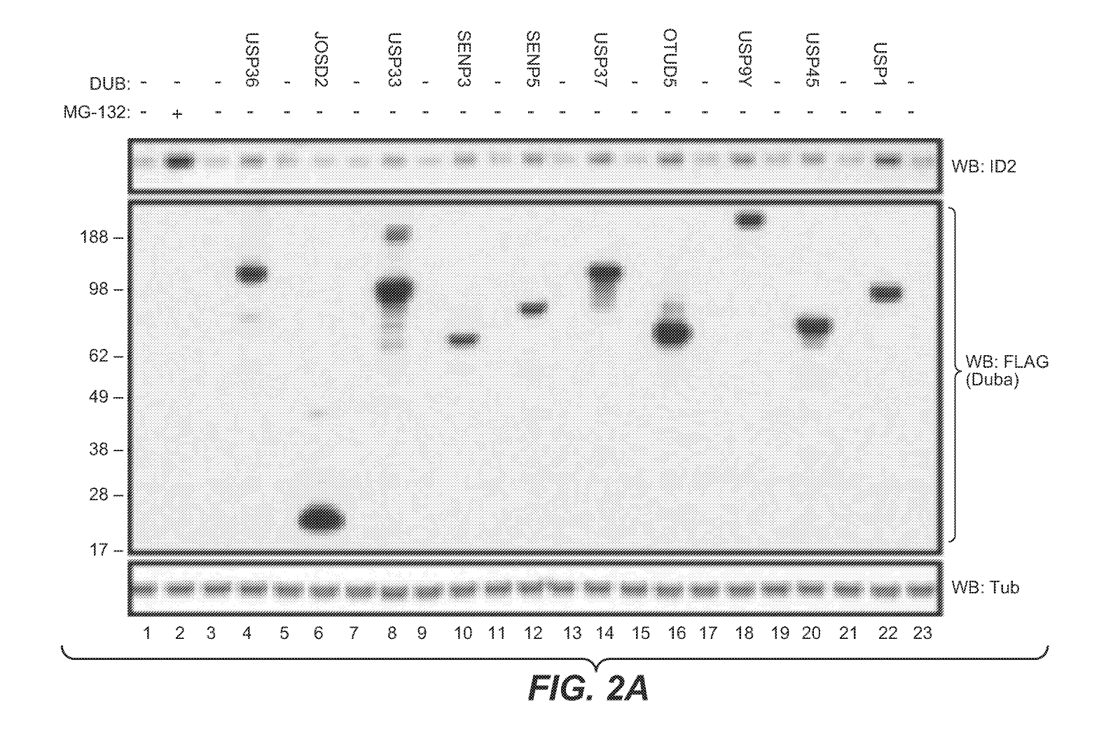

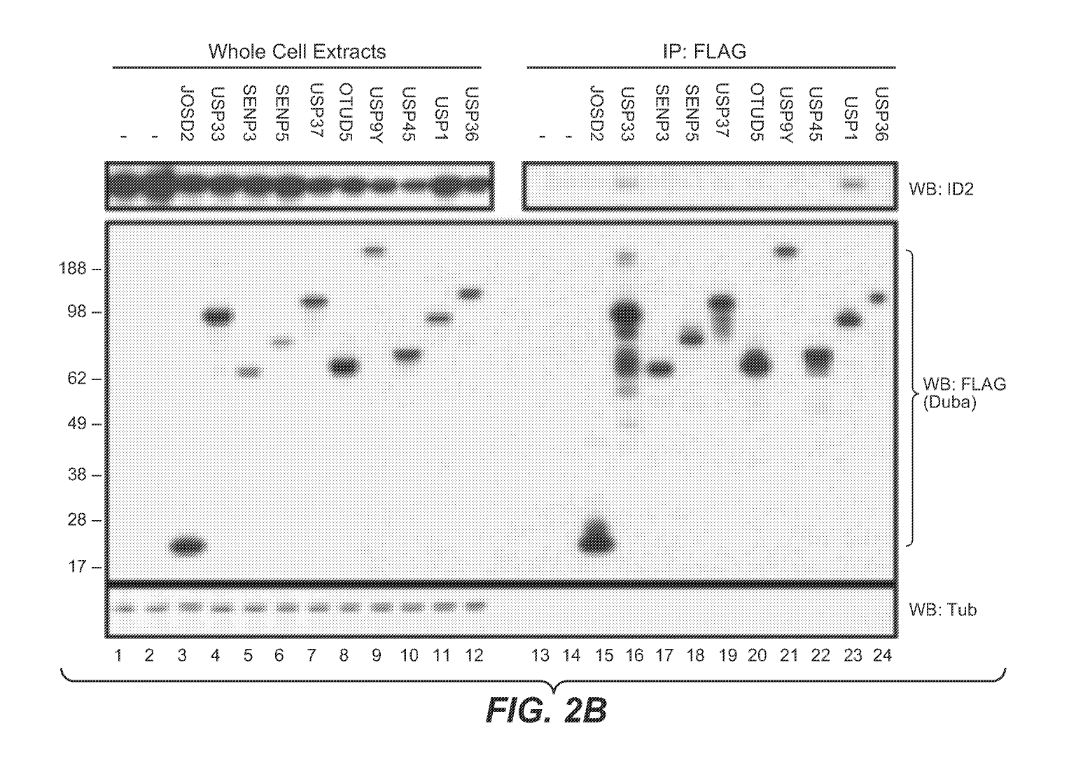

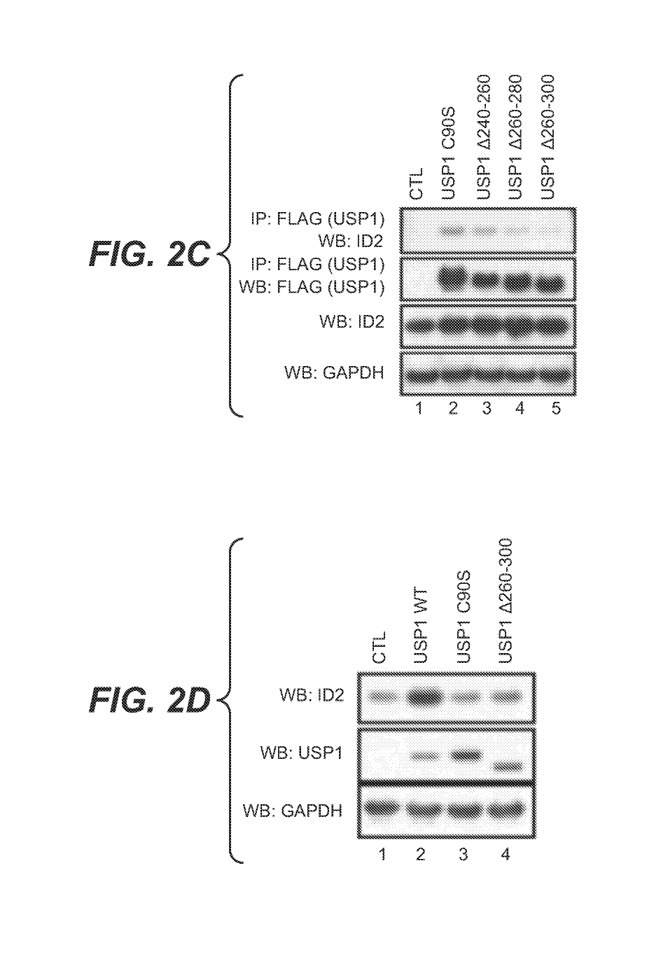

To identify deubiquitinases (DUBs) that stabilize ID proteins, 94 human DUBs with C-terminal Flag epitopes were overexpressed in 293T cells and assessed endogenous ID2 abundance by western blot (see Table S1 available online). 293T cells degrade ID2 in a proteasome-dependent manner because ID2 accumulates after treatment with the proteasome inhibitor MG-132 (FIG. 2A). DUBs that increased endogenous ID2 were USP36, USP33, SENP3, SENP5, USP37, OTUD5, USP9Y, USP45, and USP1 (FIG. 2A). To exclude indirect mechanisms increasing ID2 expression, ID2-interacting DUBs USP1 and USP33 were focused on. (FIG. 2B). However, unlike USP1, USP33 lacked deubiquitinating activity against ID2 (data not shown).

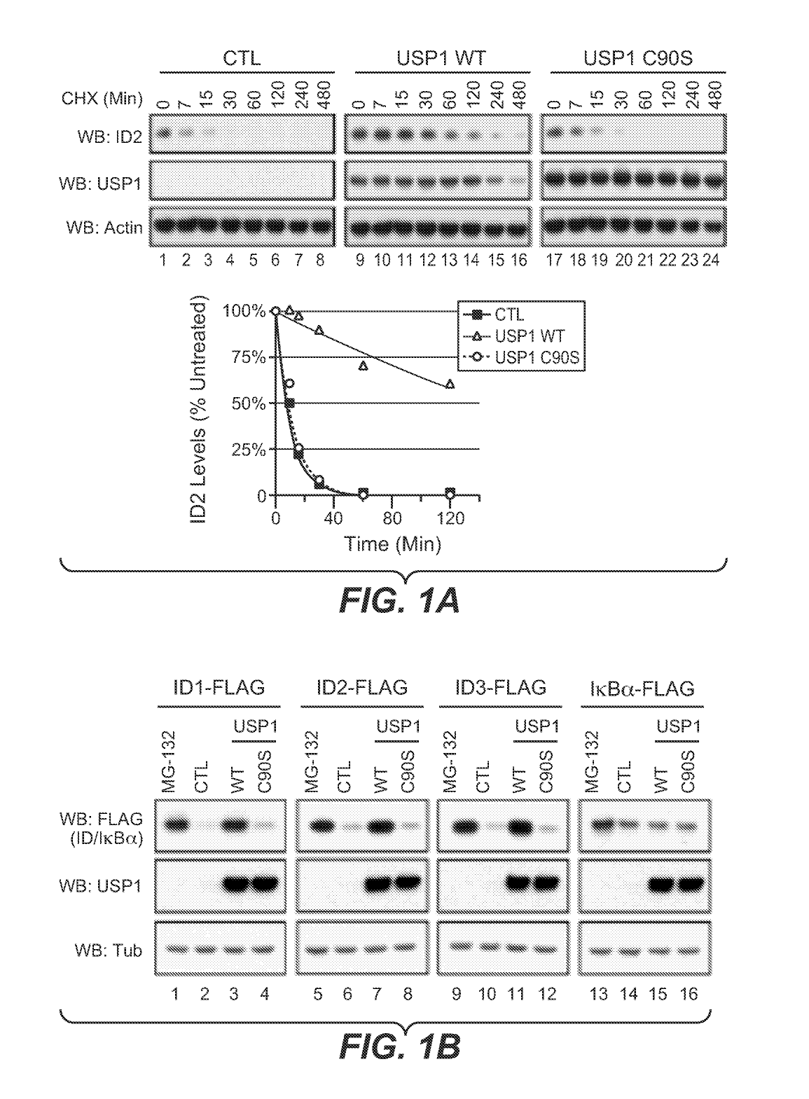

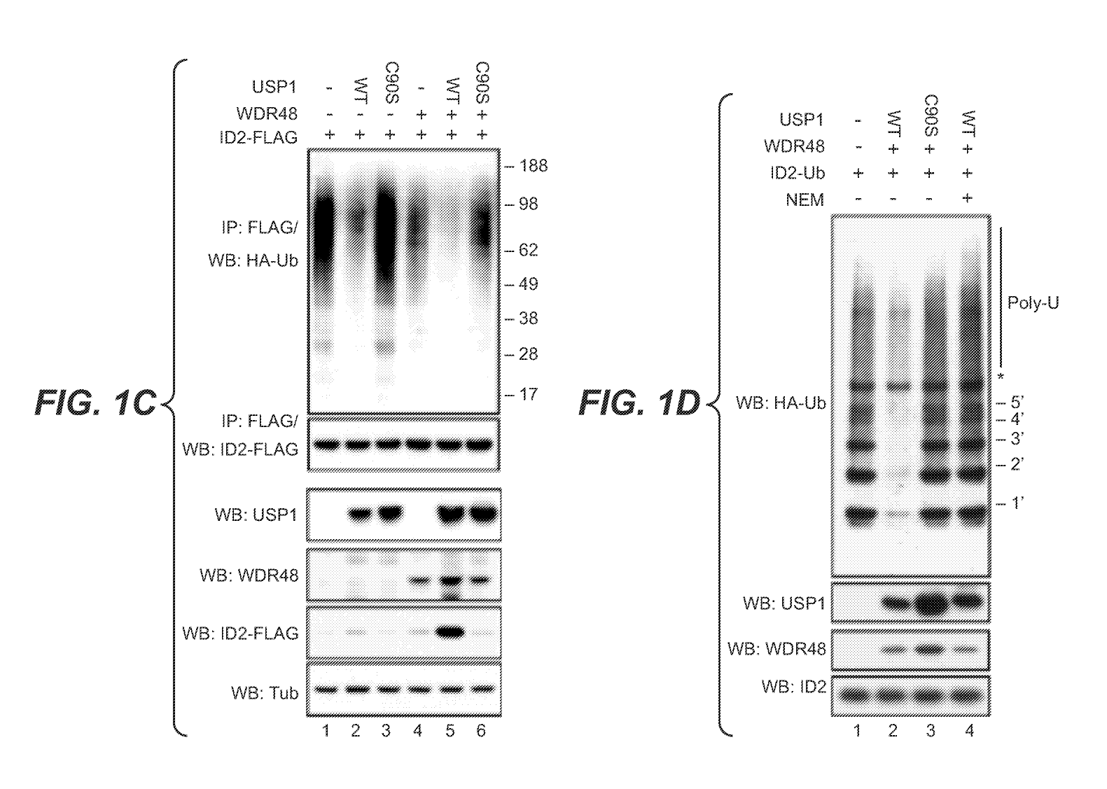

To determine whether USP1 extended the half-life of ID2, 293T cells were transfected with USP1 and monitored ID2 abundance after treatment with the translational inhibitor cycloheximide. In the absence of new protein synthesis, ID2 was cleared rapidly from cells transfected with a control vector, with a half-life of approximately 2 min (FIG. 1A). Overexpressed USP1 extended the half-life of ID2 to over 80 min. The proteolytic activity of USP1 was necessary for ID2 accumulation because the catalytically inactive point mutant USP1 C90S (Nijman et al., 2005) neither increased the half-life of ID2 (FIG. 1A) nor altered ID2 steady-state abundance (FIG. 1B). Similar results were obtained with ID1 and ID3. USP1 appeared to target IDs specifically because it did not enhance expression of labile IkBa (Palombella et al., 1994).

Next, the effect of USP1 on ID2 ubiquitination was accessed. Wild-type USP1, but not USP1 C905, reduced the amount of ID2 modified with HA-tagged ubiquitin (FIG. 1C). Both basal and USP1-induced ID2 deubiquitination was enhanced by coexpression of USP1 cofactor WDR48 (Cohn et al., 2007) (FIG. 1C). To address whether USP1 deubiquitinated ID2 directly, ubiquitinated ID2 purified from 293T cells was incubated in vitro with either wild-type USP1 or USP1 C90S purified separately from 293T cells. Ubiquitinated ID2 was decreased by wild-type USP1 but not USP1 C90S (FIG. 1D), indicating that deubiquitination was unlikely a consequence of a coeluted protease. The decrease in ID2 ubiquitination also was sensitive to N-ethylmaleimide, confirming the involvement of a cysteine protease (FIG. 1D). Consistent with ubiquitinated ID2 being a USP1 substrate, deletion mutant USP1D260-300 interacted poorly with ID2 (FIG. 2C) and did not enhance ID2 abundance (FIG. 2D).

### USP1 and ID2 are Coordinately Overexpressed in a Subset of Primary Osteosarcoma Tumors

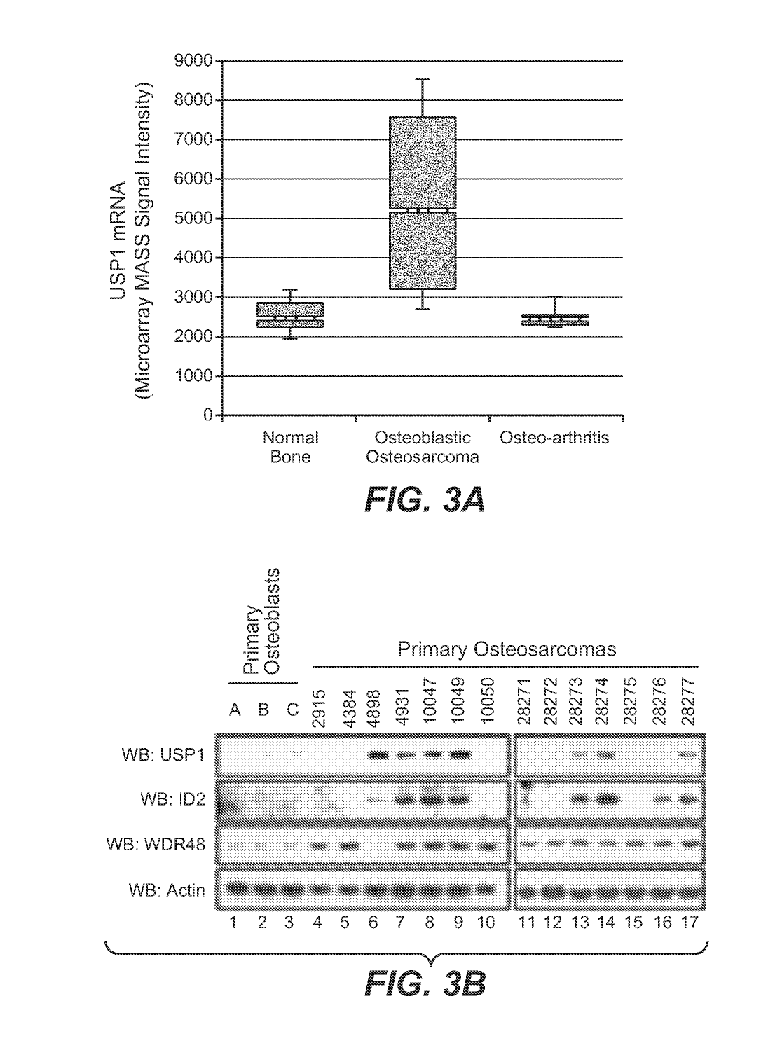

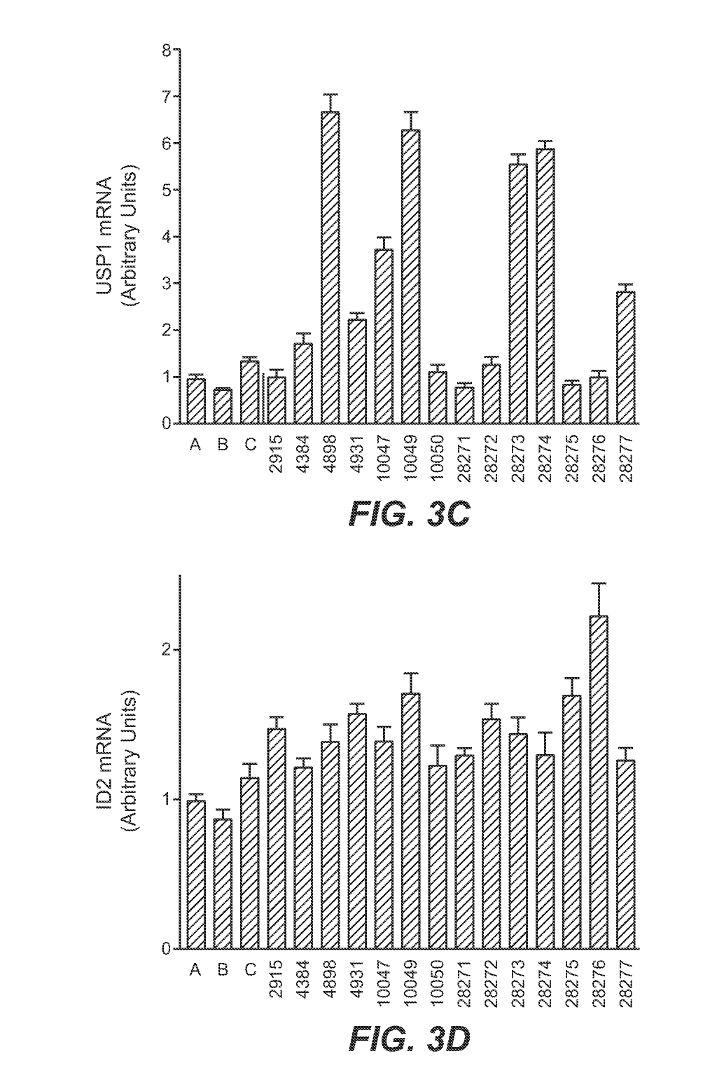

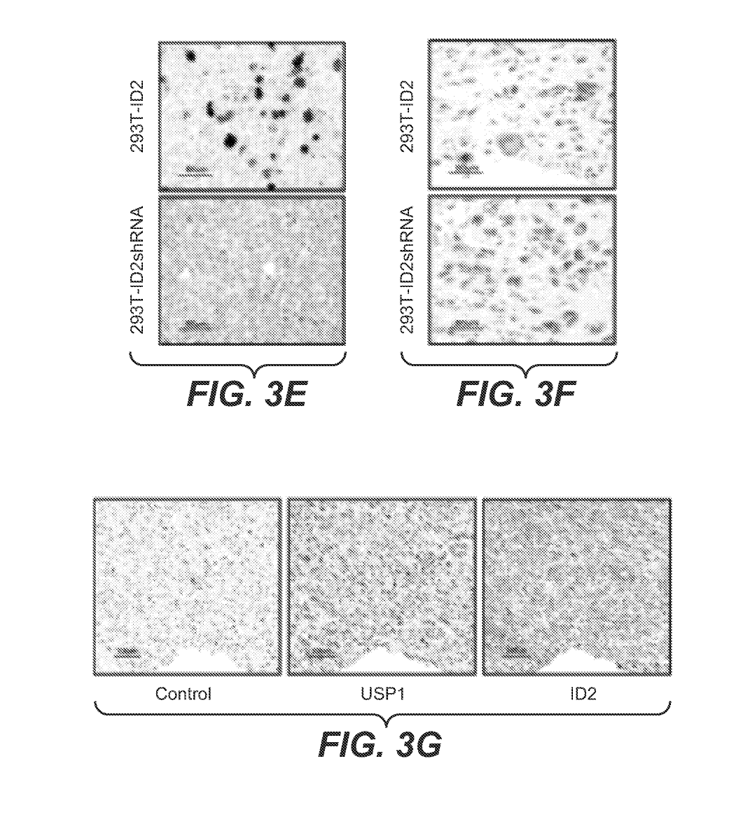

To identify a biological context in which USP1 deubiquitinates ID proteins, USP1 expression patterns were analyzed. Microarray analyses of healthy and diseased human tissues revealed that osteosarcoma tumors expressed more USP1 mRNA than healthy or osteoarthritic bone biopsies (FIG. 3A). Western blotting of a separate set of primary human osteosarcoma biopsies found that USP1 was elevated in 7 of 14 osteosarcomas when compared to 3 normal primary human osteoblast samples (FIG. 3B). Strikingly, ID2 protein abundance in these primary human tumor samples correlated well with USP1 abundance. One anomalous sample contained abundant USP1 but little ID2 (FIG. 3B, lane 6), perhaps due to poor expression of the USP1 cofactor WDR48. Another sample contained abundant ID2 and little USP1 (FIG. 3B, lane 16), which may reflect reduced ID2 ubiquitination or that other DUBs are active.

The amount of USP1 protein in the primary osteosarcomas correlated largely with USP1 mRNA abundance (FIG. 3C), suggesting that elevated USP1 in osteosarcoma is due to transcriptional upregulation. In contrast, ID2 protein and mRNA levels correlated poorly (FIG. 3D). The coincident overexpression of USP1 and ID2 in primary osteosarcoma was confirmed by immunohistochemistry (FIGS. 3E-3G). These results strongly suggest that USP1 modifies ID proteins posttranslationally in osteosarcoma.

### USP1 Stabilizes ID Proteins in Osteosarcoma

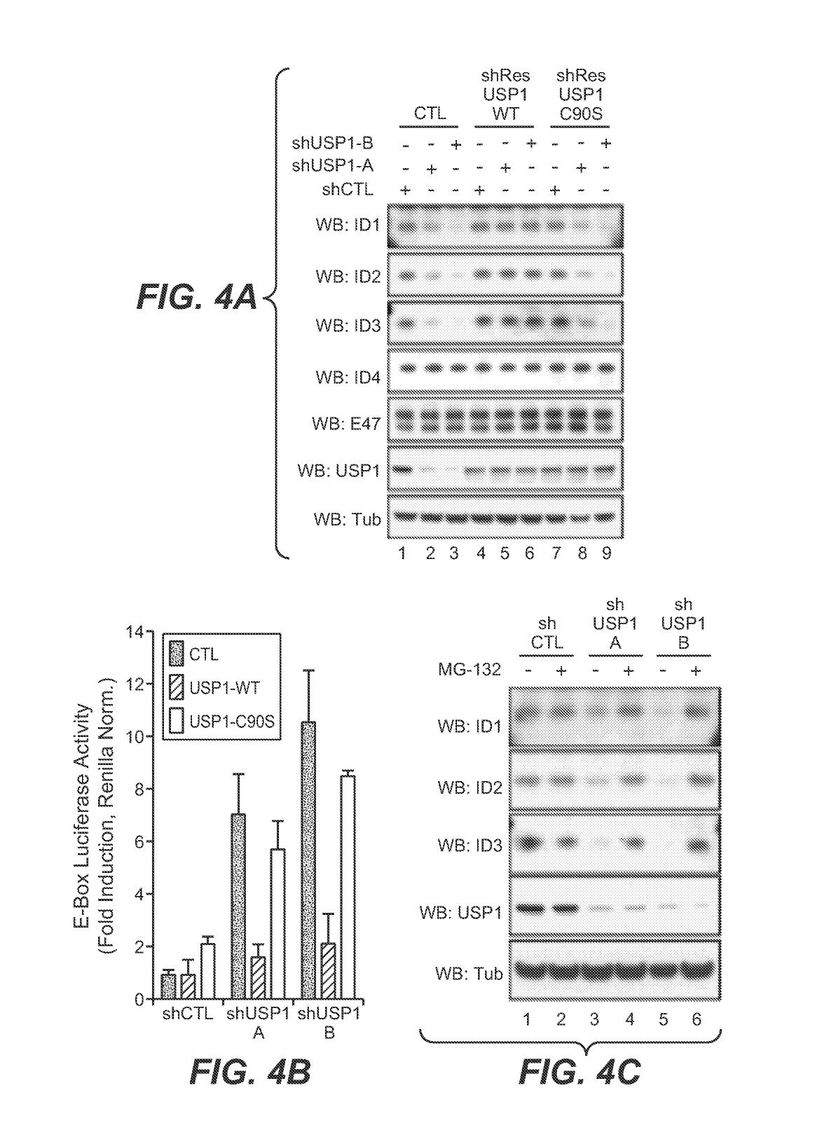

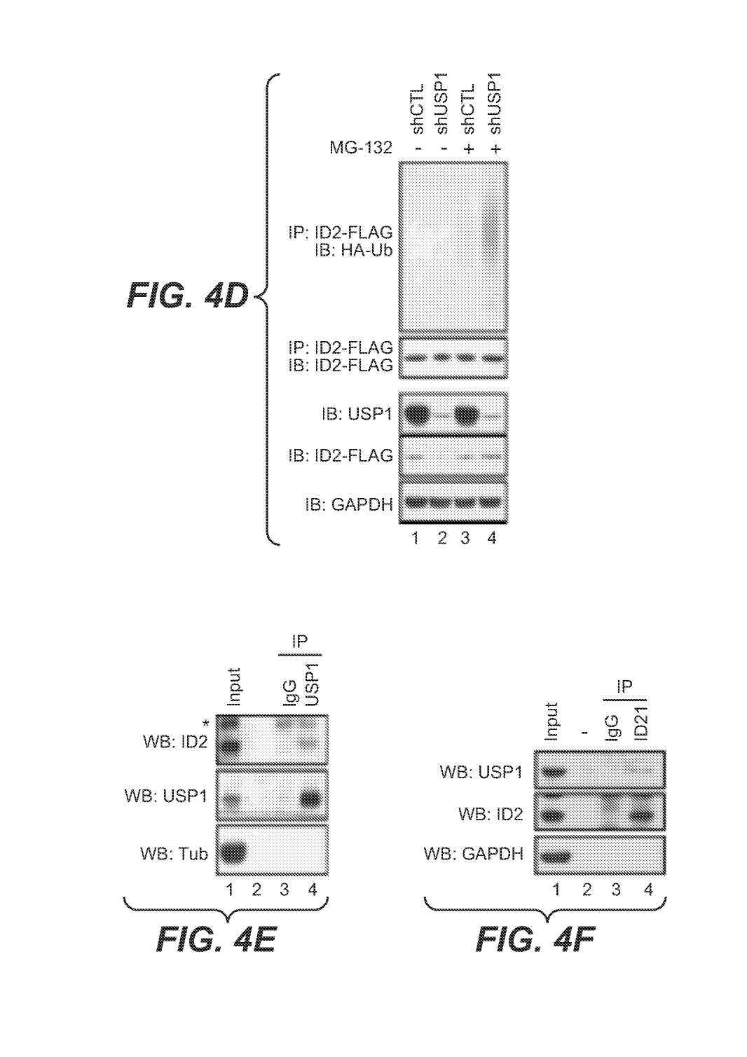

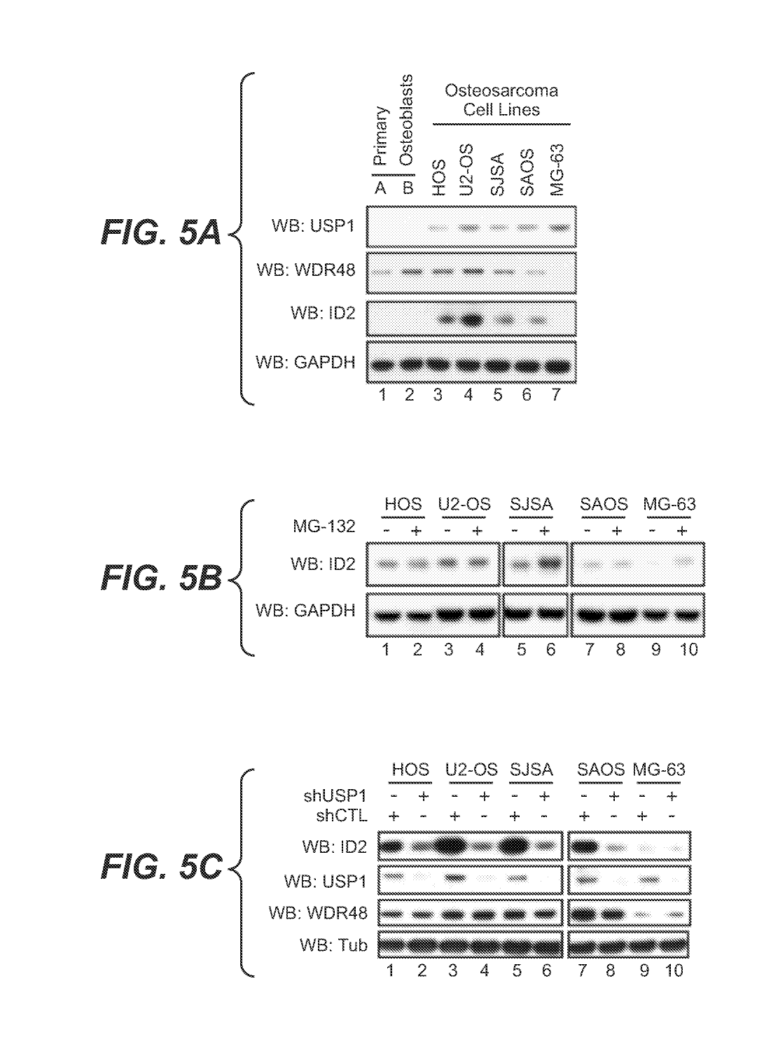

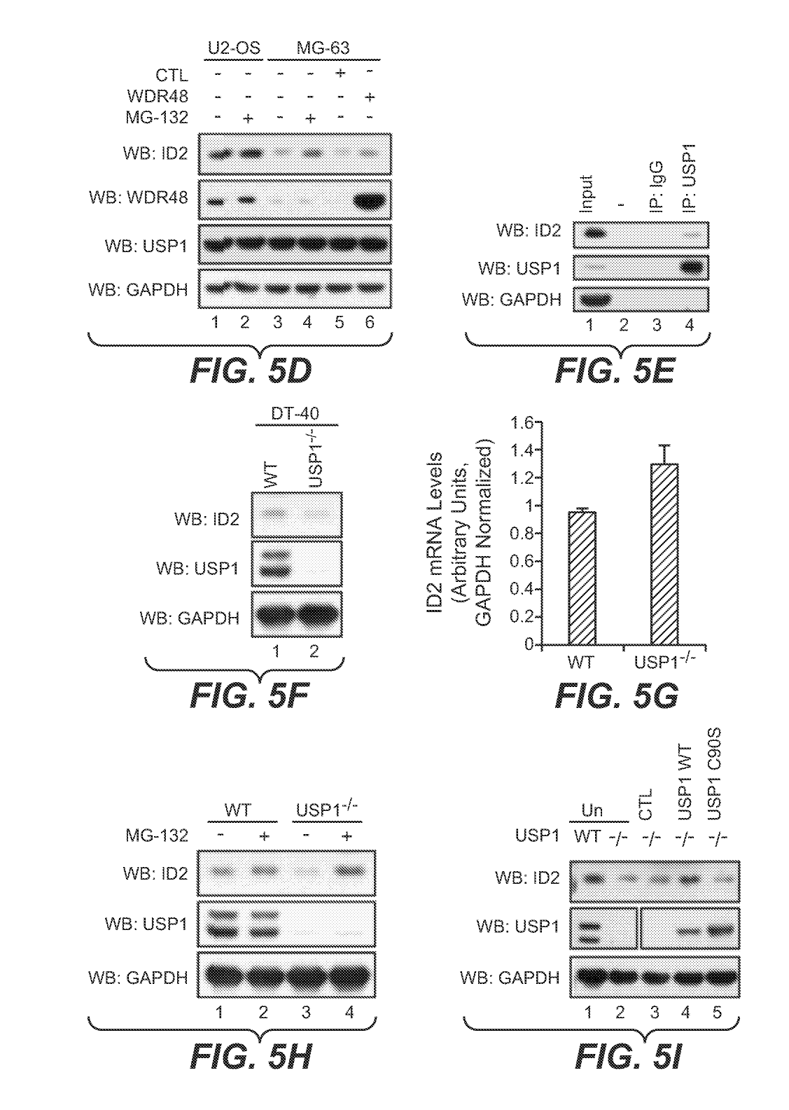

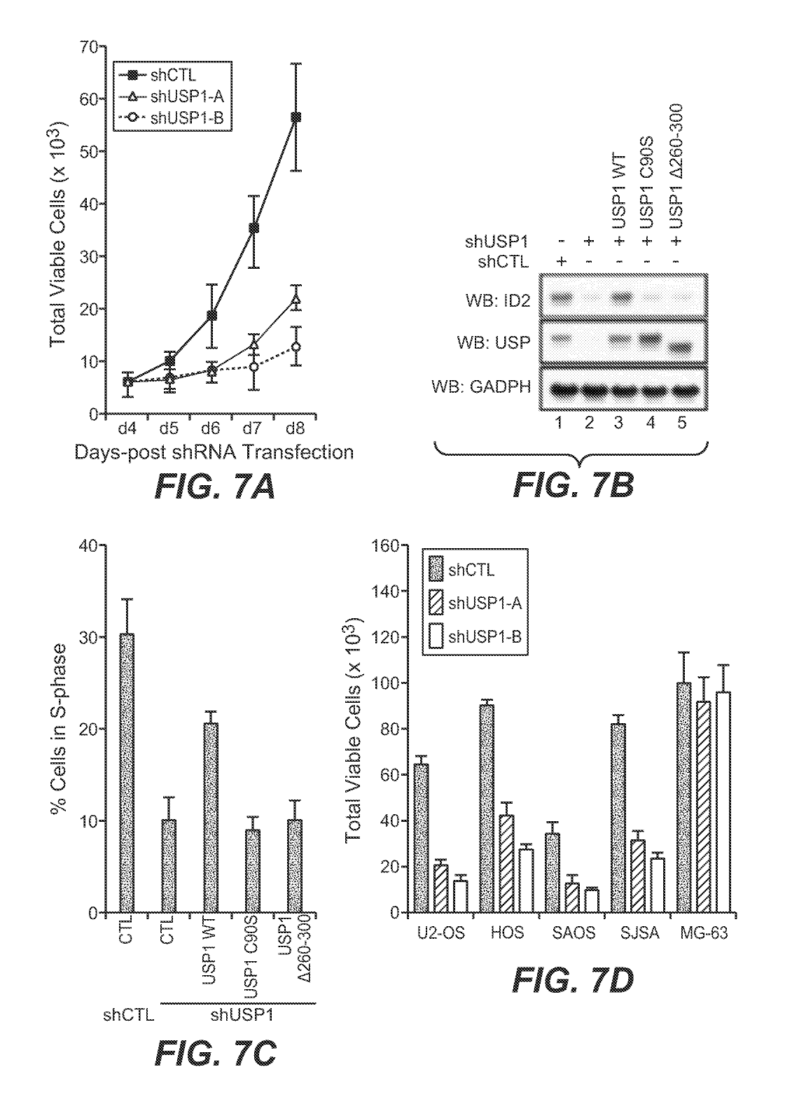

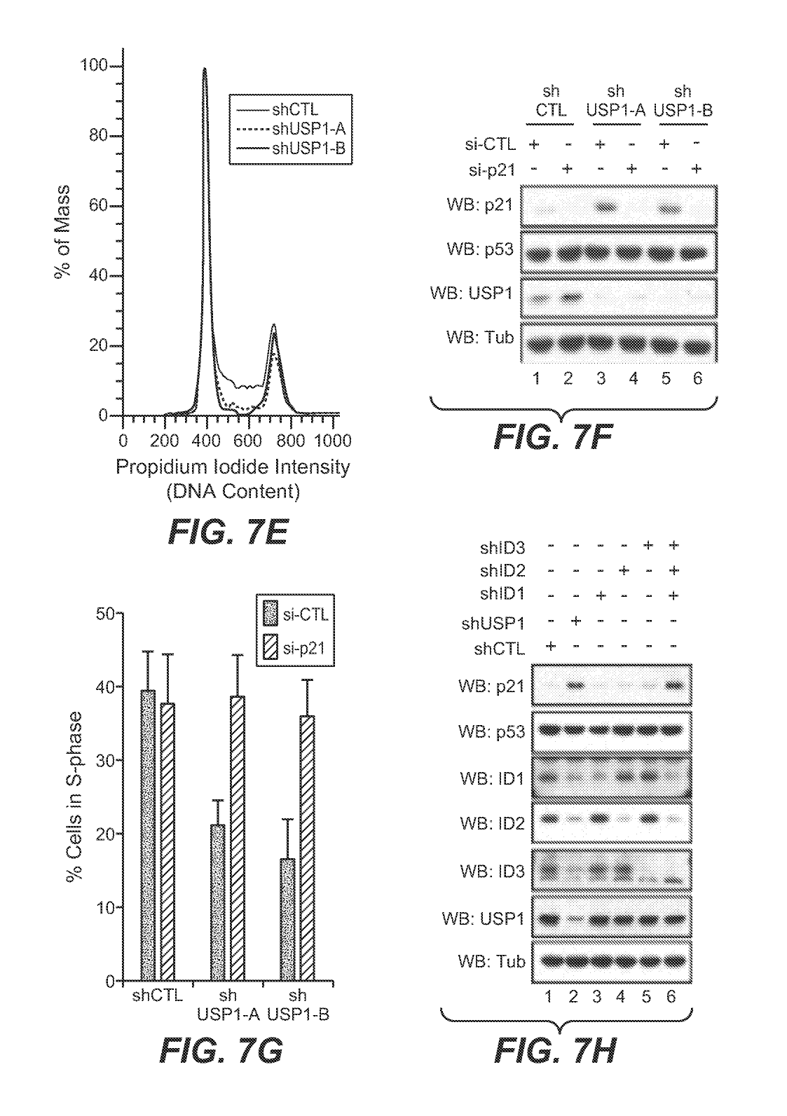

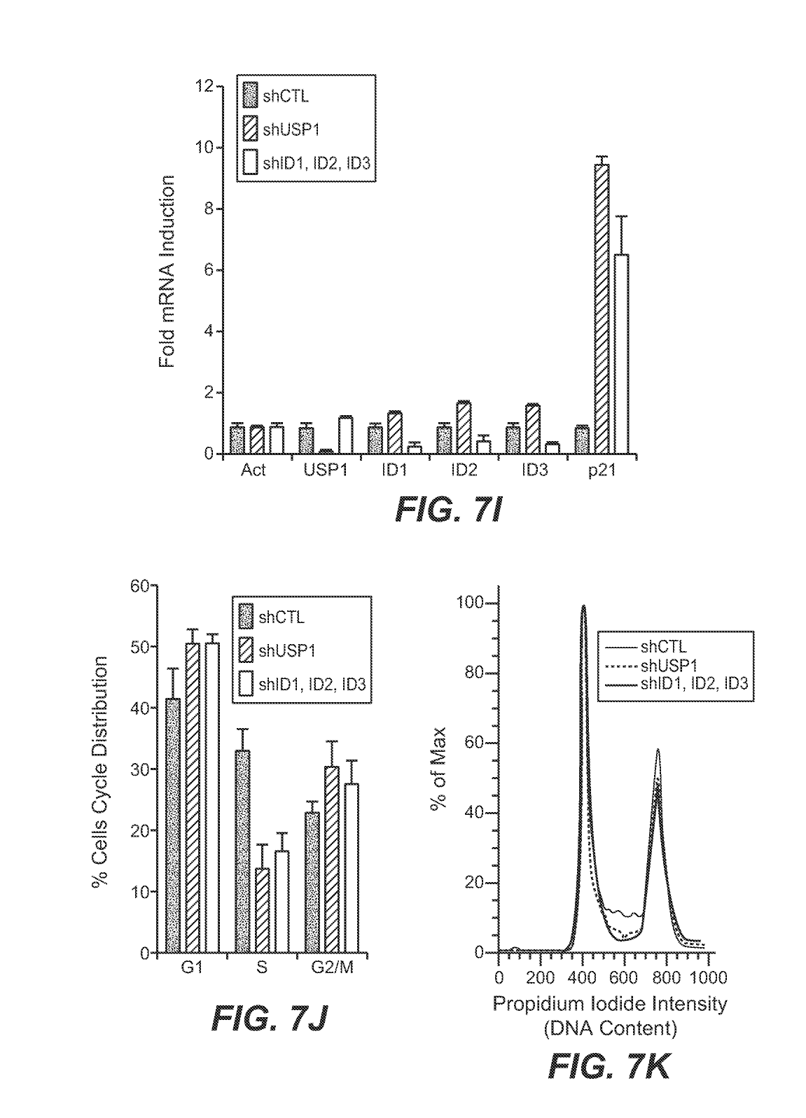

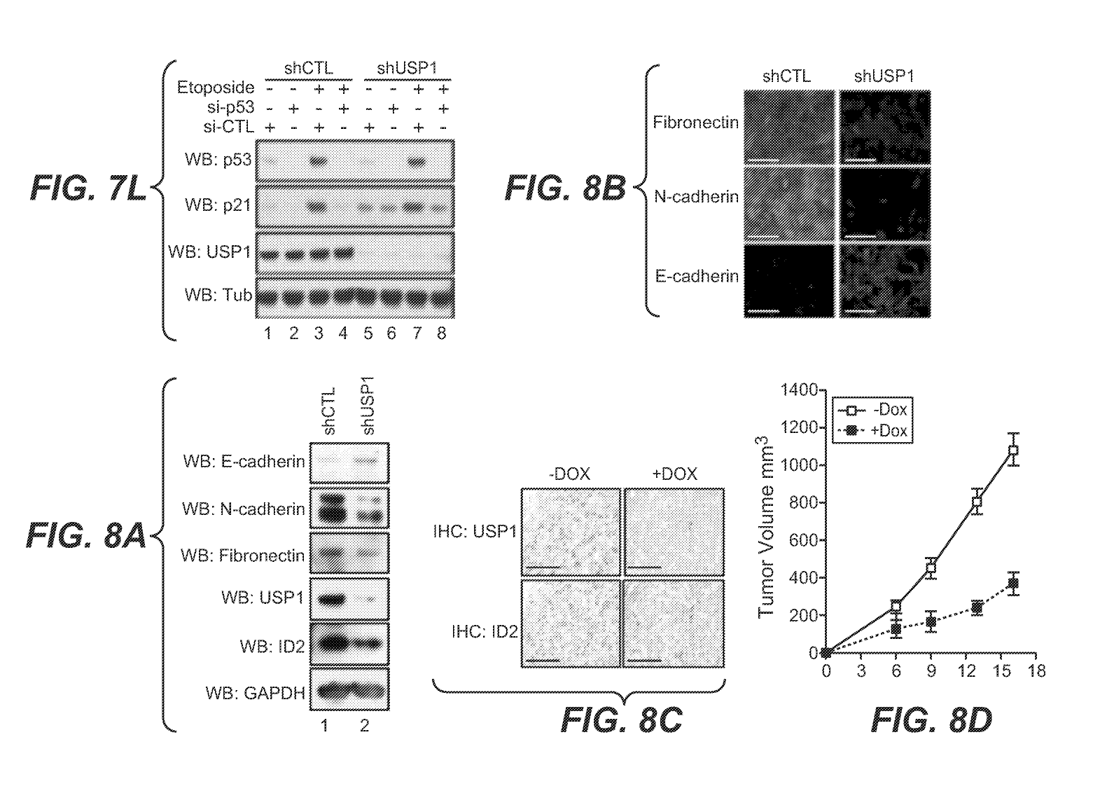

USP1 abundance and ID2 stability in human osteosarcoma cell lines and in primary osteoblasts was also assessed (FIG. 5A). In U2-OS osteosarcoma cells, USP1 was elevated, and the normally labile ID2 was stable (FIGS. 5A and 5B). Knockdown of USP1 with two distinct USP1 shRNAs caused a reduction in ID1, ID2, and ID3 but had no effect on ID4 (FIG. 4A). ID1, ID2, and ID3 mRNAs were not reduced, excluding decreased transcription as the reason for the drop in ID protein abundance (FIG. 7I). USP1 knockdown specificity was confirmed with shRNA-resistant USP1, which restored ID1, ID2, and ID3 to basal levels. USP1 catalytic activity was essential for ID stability because shRNA-resistant USP1 C90S did not restore ID protein levels. Similar results were observed in osteosarcoma cell lines HOS, SAGS, and SJSA (FIG. 5C). USP1 knockdown did not impact ID2 abundance in MG-63 osteosarcoma cells, likely because these cells express very little WDR48 (FIG. 5C). Consistent with WDR48 deficiency limiting USP1 activity in MG-63 cells, ectopic WDR48 increased ID2 (FIG. 5D).

To determine if USP1 knockdown and decreased IDs 1-3 modulated bHLH transcriptional activity, U2-OS cells were transfected with an E box-driven luciferase reporter gene. USP1 shRNAs enhanced expression of this reporter 7- to 10-fold over a control shRNA, consistent with activation of bHLH proteins when ID proteins are decreased (FIG. 4B). shRNA-resistant wild-type USP1, but not USP1 C905, suppressed E box-driven reporter activity caused by USP1 knockdown, confirming that USP1 catalytic activity is required for bHLH-dependent transcription as well as ID protein stabilization.

The acute loss of IDs 1-3 following knockdown of endogenous USP1 suggested ID protein destabilization via proteasomemediated degradation. As anticipated, the proteasome inhibitor MG-132 did not by itself alter ID protein abundance in U2-OS cells, suggesting that the IDs are intrinsically stable in cells that express USP1 highly (FIG. 4C). However, MG-132 treatment did restore ID expression after USP1 knockdown, indicating that ID proteins are subject to proteasome-mediated clearance upon USP1 depletion. In keeping with this scenario, USP1 knockdown in MG-132-treated U2-OS cells increased the amount of ubiquitinated ID2 (FIG. 4D).

Next, endogenous USP1 was confirmed to associate with an endogenous ID in osteosarcoma cells. ID2 coimmunoprecipitated with endogenous USP1 from U2-OS cells (FIG. 4E), albeit not with 1:1 stoichiometry, but this is to be expected for a transient enzyme-substrate interaction. Similar results were obtained in HOS cells (FIG. 5E). USP1 also coimmunoprecipitated with ID2 (FIG. 4F). Collectively, theses results suggest that USP1 is a potent DUB and stabilizing factor for ID1, ID2, and ID3 in osteosarcoma.

ID2 stabilization by USP1 was not limited to the setting of osteosarcoma. USP1^{-/-} DT40 chicken B cells (Oestergaard et al., 2007) expressed less ID2 protein than their wild-type counterparts (FIG. 5F) despite expressing similar levels of ID2 mRNA (FIG. 5G). Consistent with USP1 deubiquitinating and stabilizing ID2, proteasome inhibition with MG-132 increased ID2 in USP1^{-/-}, but not wild-type, DT40 cells (FIG. 5H). In addition, USP1^{-/-} DT40 cells reconstituted with wild-type USP1, but not USP1 C905, contained equivalent ID2 to wild-type DT40 cells (FIG. 5I).

USP1 Suppresses p21-Mediated Cell-Cycle Arrest in Osteosarcoma

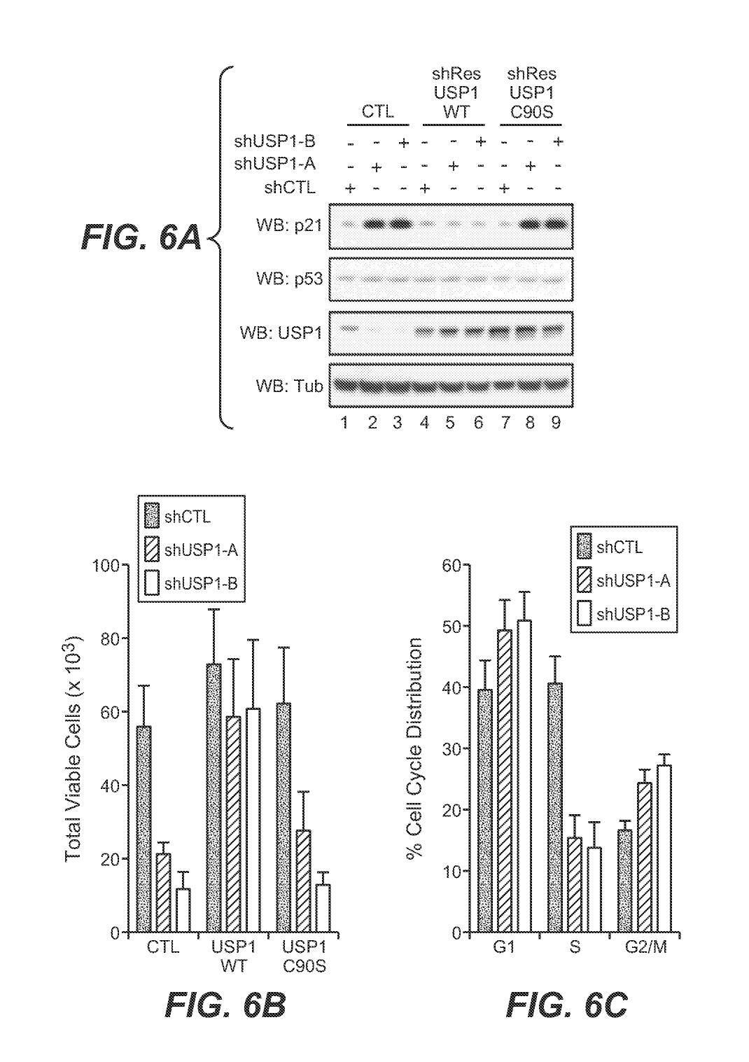

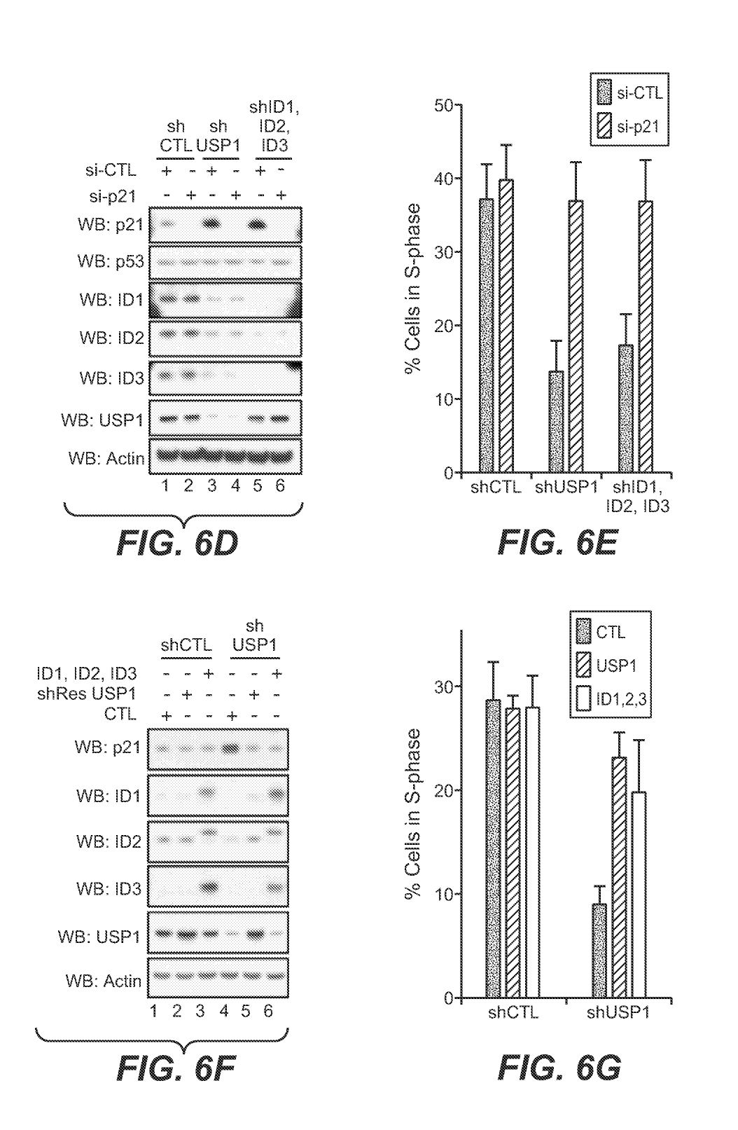

One potential consequence of USP1 deficiency and increased bHLH transcriptional activity in osteosarcoma cells is induction of bHLH-regulated CDKI p21. Indeed, p21 was increased in U2-OS cells transfected with USP1 shRNAs relative to cells transfected with a control shRNA (FIG. 6A). shRNA-resistant wild-type USP1, but not USP1 C905, reduced p21 to levels observed in control cells, confirming knockdown specificity in this setting. The tumor suppressor p53, a well-known inducer of CDKN1A, was not increased by USP1 knockdown, suggesting that increased p21 was p53 independent.

p21 is a potent inhibitor of cell cycle progression (Polyak et al., 1996), so the proliferative capacity of U2-OS cells following USP1 knockdown was assessed. Consistent with increased p21, USP1 knockdown reduced U2-OS cell proliferation (FIG. 6B and FIG. 7A). shRNA-resistant wild-type USP1, but neither USP1 C90S nor USP1D260-300, restored cell proliferation (FIGS. 7B and 7C), indicating that both USP1 catalytic activity and ID substrate recognition are required to maintain U2-OS cell proliferation. USP1 knockdown similarly reduced proliferation in

HOS, SAGS, and SJSA, but not MG-63 osteosarcoma cells (FIG. 7D). Flow cytometric analysis of the DNA content in U2-OS cells after USP1 knockdown revealed a moderate increase in cells in G1 and G2 phases of the cell cycle with a pronounced reduction of cells in S phase (FIG. 6C and FIG. 7E). Apoptosis induction following USP1 knockdown was not prominent; few cells with a subdiploid DNA content were observed, there was no increase in cells stained with annexin V, and increased processing of caspase-3 was not detected (FIG. 7E; data not shown). Significantly, CDKN1A siRNAs restored S phase entry in USP1-deficient U2-OS cells (FIGS. 7F and 7G), indicating that p21 is essential for the cell-cycle arrest induced by USP 1 knockdown.

### USP1 Regulates p21 Expression and Cell-Cycle Arrest in Osteosarcoma Via ID Proteins

If ID degradation in the absence of USP1 caused p21 induction, then knockdown of the ID proteins should phenocopy USP1 knockdown. shRNA knockdown of IDs 1-3 individually did not alter p21 levels, but combined knockdown of ID1, ID2, and ID3 increased p21 similar to USP1 knockdown (FIG. 7H). ID and USP1-deficient cells also expressed comparable levels of CDKN1A mRNA (FIG. 7I). Consistent with these observations, ID deficiency caused cell-cycle arrest similar to USP1 deficiency (FIGS. S3J and S3K), and this was rescued by p21 knockdown (FIGS. 6D and 6E).

CDKN1A is regulated by many transcription factors, including p53, which is activated in response to DNA damage (Kastan et al., 1991). p53 knockdown inhibited etoposide-induced p21 in U2-OS cells but did not block the increase in p21 protein seen after USP1 knockdown (FIG. 7L), supporting a p53-independent mechanism of p21 induction. Because USP1 is reported to target PCNA and FANCD2 during DNA repair (Nijman et al., 2005; Huang et al., 2006), production of DNA damage as a result USP1 knockdown was determined. H2AX phosphorylation that is associated with DNA damage (Rogakou et al., 1999) increased after etoposide treatment but not USP1 knockdown (data not shown). These observations, combined with the ability of USP1 shRNAs to arrest p53-deficient SAOS cells (FIG. 7D), exclude general DNA damage, and p53 in particular, as intermediaries in p21 induction following USP1 knockdown.

USP1 was confirmed to regulate p21 expression and cell cycling via the IDs by rescuing the effects of USP1 knockdown in U2-OS cells with ectopic expression of ID1, ID2, and ID3. ID expression in USP1-depleted cells inhibited p21 expression (FIG. 6F) and blocked cell-cycle arrest (FIG. 6G). Taken together, the results demonstrate that USP1 suppresses p21 via ID protein stabilization and inhibition of bHLH transcriptional activity in osteosarcoma.

### USP1 and ID Proteins Restrict Osteogenic Commitment in Osteosarcoma

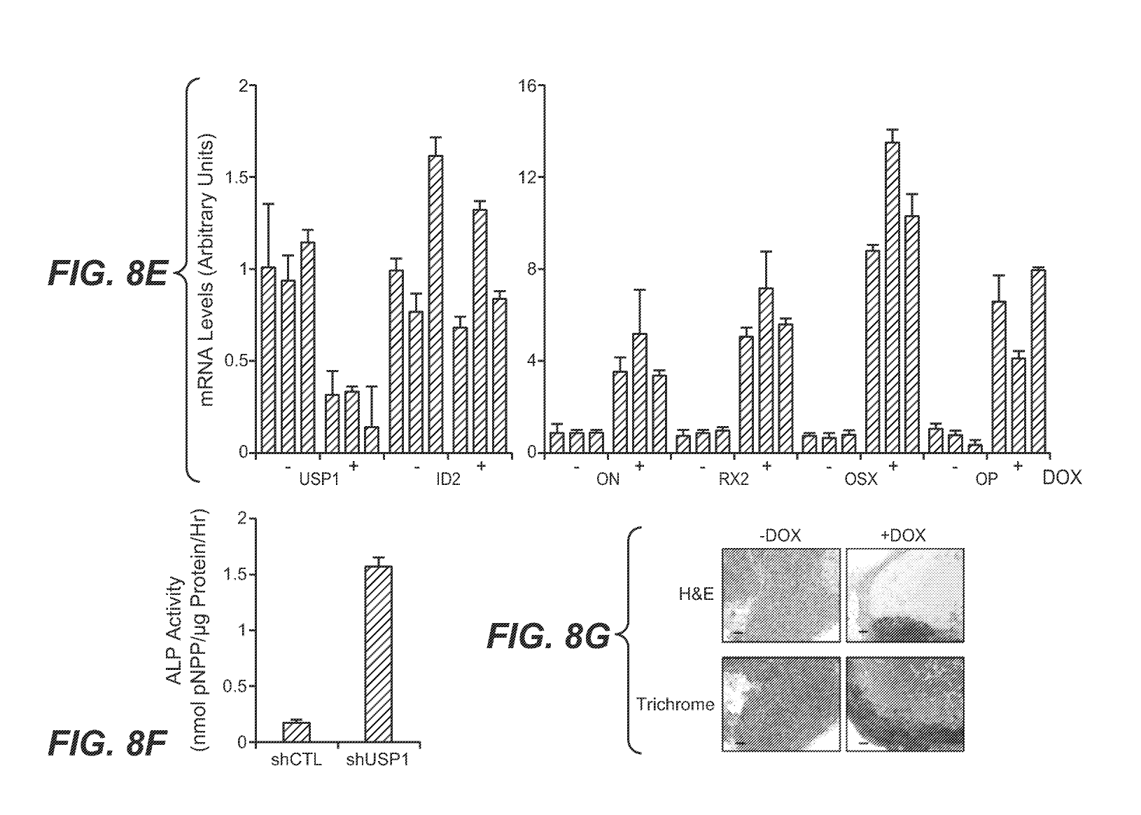

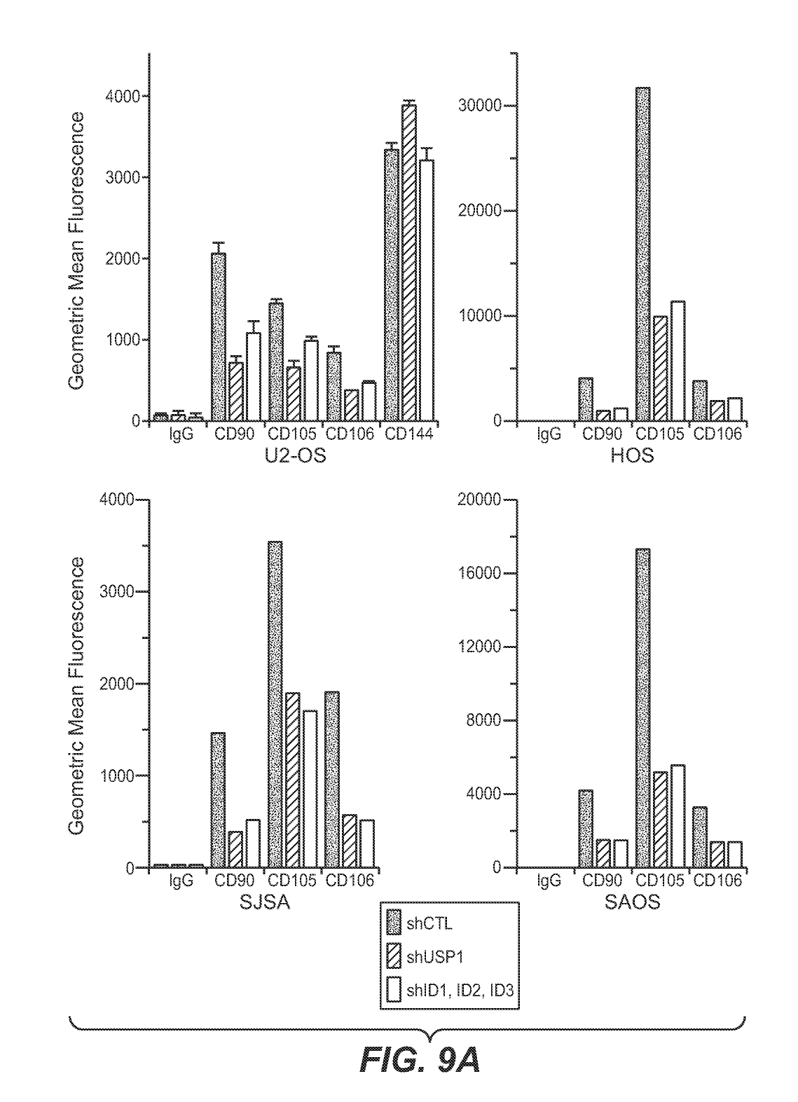

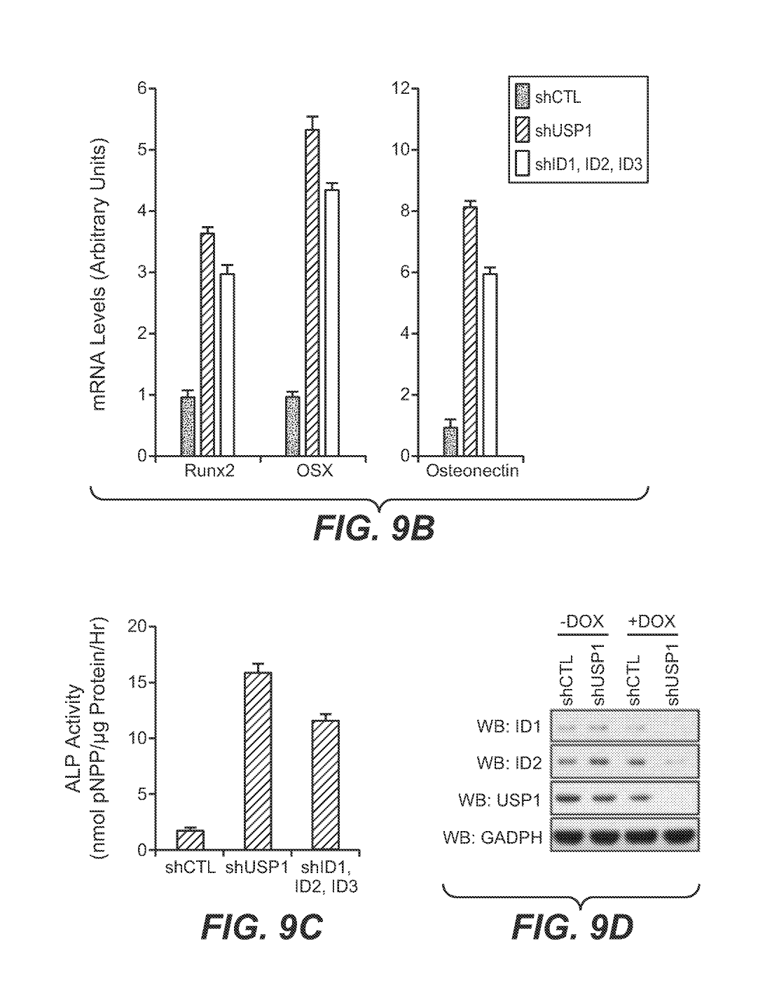

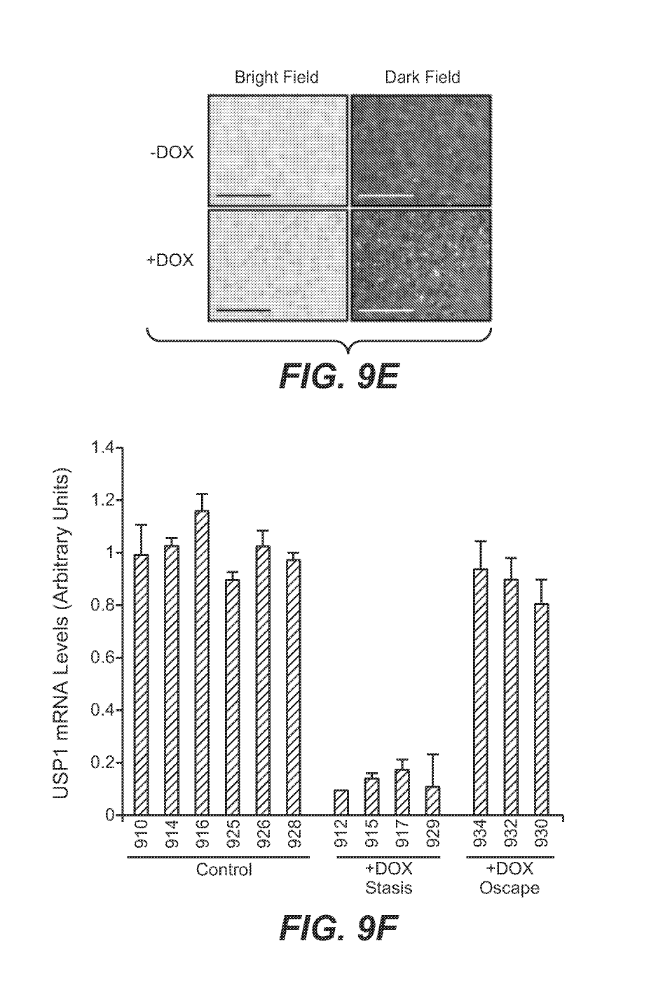

Osteosarcomas are heterogeneous tumors comprised of disorganized masses of osteoblasts, chondrocytes, and adipocytes. These tumors are thought to develop from a mesenchymal stem cell population that can give rise to all three lineages (Tang et al., 2008). Accordingly, osteosarcoma cell lines fail to express classical osteoblast markers such as RUNX2, OSTERIX, SPARC/OSTEONECTIN, and alkaline phosphatase (ALP) (Luo et al., 2008). Osteosarcoma cell lines also express surfacemarkers characteristic of mesenchymal stem cells, including CD90, CD105, and CD106 (Di Fiore et al., 2009). In light of the role that IDs play in stem cell maintenance and regulation of differentiation, triggering of osteoblastic differentiation by either USP1 or ID knockdown in osteosarcoma was investigated. U2-OS cells transfected with USP1 or ID shRNAs expressed less CD105, CD106, and CD90 relative to control cells, whereas all cells expressed equivalent amounts of the unrelated surface marker CD 144 (FIG. 9A). Similar results were observed with HOS, SJSA, and SAOS cell lines. USP1 or ID knockdown also increased expression of osteoblastic RUNX2, OSTERIX, and OSTEONECTIN (FIG. 9B), and increased ALP activity (FIG. 9C). Increased E-cadherin expression and reduced N-cadherin and fibronectin following USP1 knockdown in U2-OS cells indicated a reversal of the epithelial to mesenchymal transition that accompanies the malignant state of osteosarcoma (FIGS. 8A and 8B) (Thiery et al., 2009). Collectively, these data suggest that ID protein stabilization by USP1 in osteosarcoma blocks a normal osteogenic differentiation program.

The potential of USP1 inhibition as a tumor differentiation strategy was investigated in the 143B osteosarcoma xenograft model. A doxycycline-induced USP1 shRNA suppressed USP1 expression and reduced ID1 and ID2 in the xenografts (FIG. 8C and FIG. 9D). ID3 was not detectable in this setting (data not shown). USP1 knockdown also reduced 143B tumor growth (FIG. 8D), promoted OSTEONECTIN, RUNX2, SPP1/OSTEOPONTIN, OSTERIX, and BGLAP/OSTEOCALCIN expression (FIG. 8E and FIG. 9E), and enhanced ALP activity (FIG. 8F). Remarkably, four of ten USP1-deficient xenograft tumors achieved stasis and differentiation in situ, displaying markedly altered cellular morphology and accumulation of acellular collagenous masses consistent with proto-ossification (FIG. 8G). The tumors that continued to proliferate showed evidence of escape from knockdown, presumably due to loss or silencing of the shRNA (FIG. 9F). These data indicate that reducing USP1 is sufficient to initiate an osteogenic differentiation program in osteosarcoma. Dysregulated USP1 Expression Inhibits hMSC Differentiation

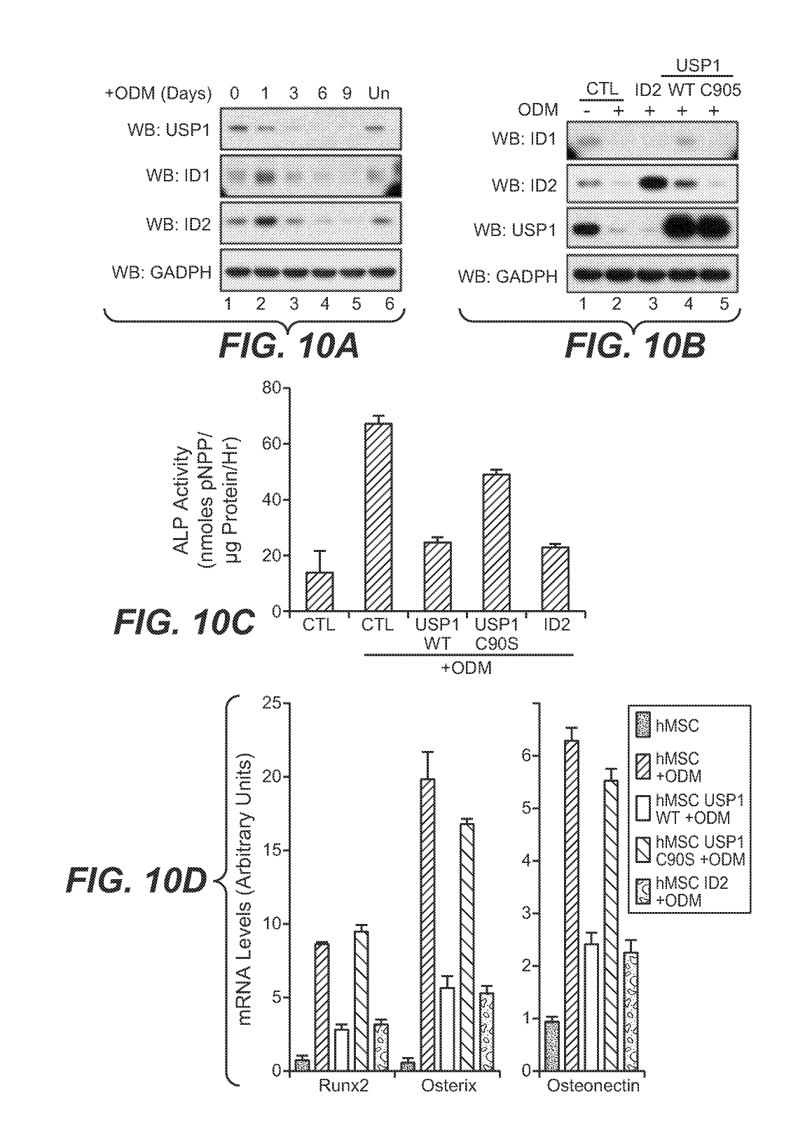

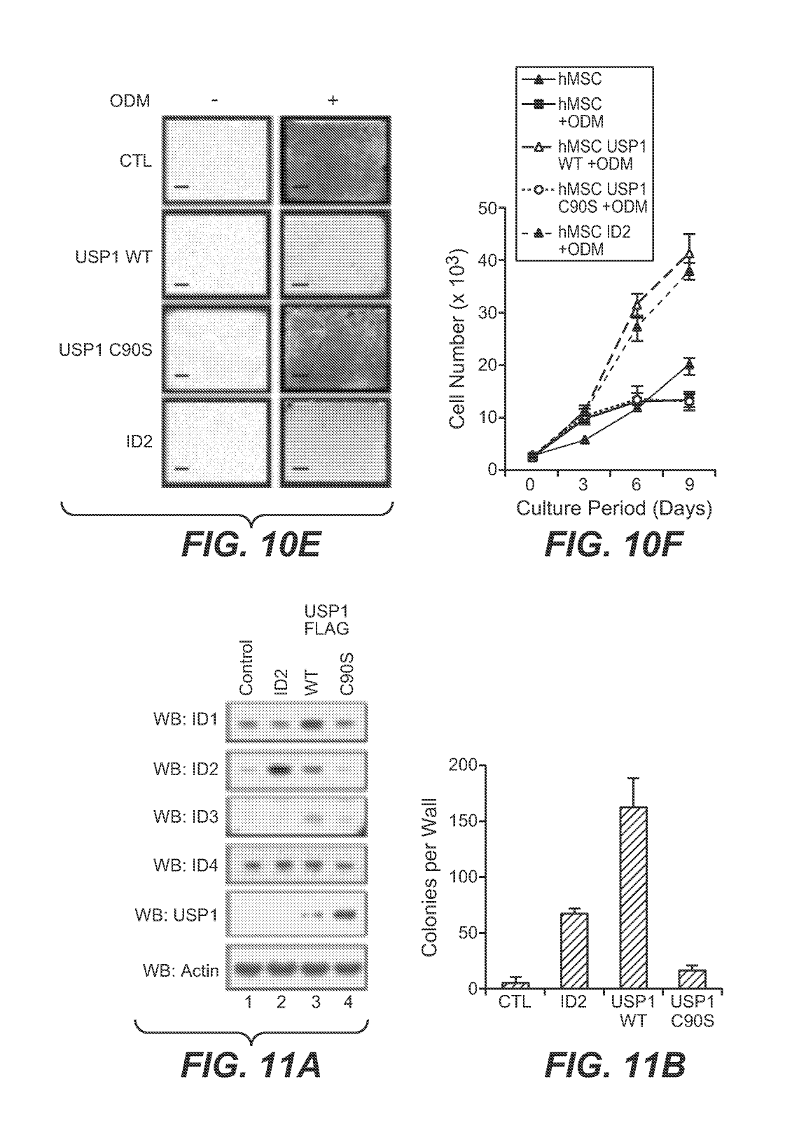

Next, it was determined whether USP1 stabilization of the IDs contributes to normal mesenchymal stem cell maintenance. USP1 was expressed in primary hMSCs but declined steadily as the cells were cultured in conditions favoring osteoblastic differentiation (FIG. 10A). Consistent with a previous study (Peng et al., 2003), ID1 and ID2 were induced transiently and then declined as well. ID3 was not detected (data not shown). These data, together with a study showing that misregulated ID expression inhibits osteogenic differentiation (Peng et al., 2004), prompted investigation as to whether USP1 overexpression disrupts hMSC differentiation. hMSCs overexpressing USP1 and cultured in osteogenic differentiation medium expressed abnormally high levels of ID1 and ID2 (FIG. 10B), exhibited low ALP activity (FIG. 10C), showed minimal induction of RUNX2, OSTERIX, and OSTEONECTIN (FIG. 10D), and stained poorly with alizarin red, which reveals mineral deposition that is a classic marker of osteoblast activity (FIG. 10E). These data imply that the hMSCs overexpressing USP1 failed to differentiate. A similar differentiation defect was observed in hMSCs overexpressing ID2, whereas hMSCs overexpressing USP1 C90S differentiated similarly to control cells. Thus, the catalytic activity of USP1 was necessary and ID stabilization sufficient to inhibit osteogenic differentiation.

Coincident with their apparent failure to differentiate, hMSCs overexpressing USP1 or ID2 proliferated significantly in the presence of excess osteogenic differentiation factors (FIG. 10F). In contrast, proliferation of control hMSCs, or those expressing USP1 C90S, slowed as they differentiated in culture. Collectively, these observations suggest that overexpression of USP1 or ID2 is sufficient to block osteoblastic differentiation, promote retention of stem-like features, and render cells resistant to differentiation cues.

### USP1 Promotes Transformation and Tumor Formation

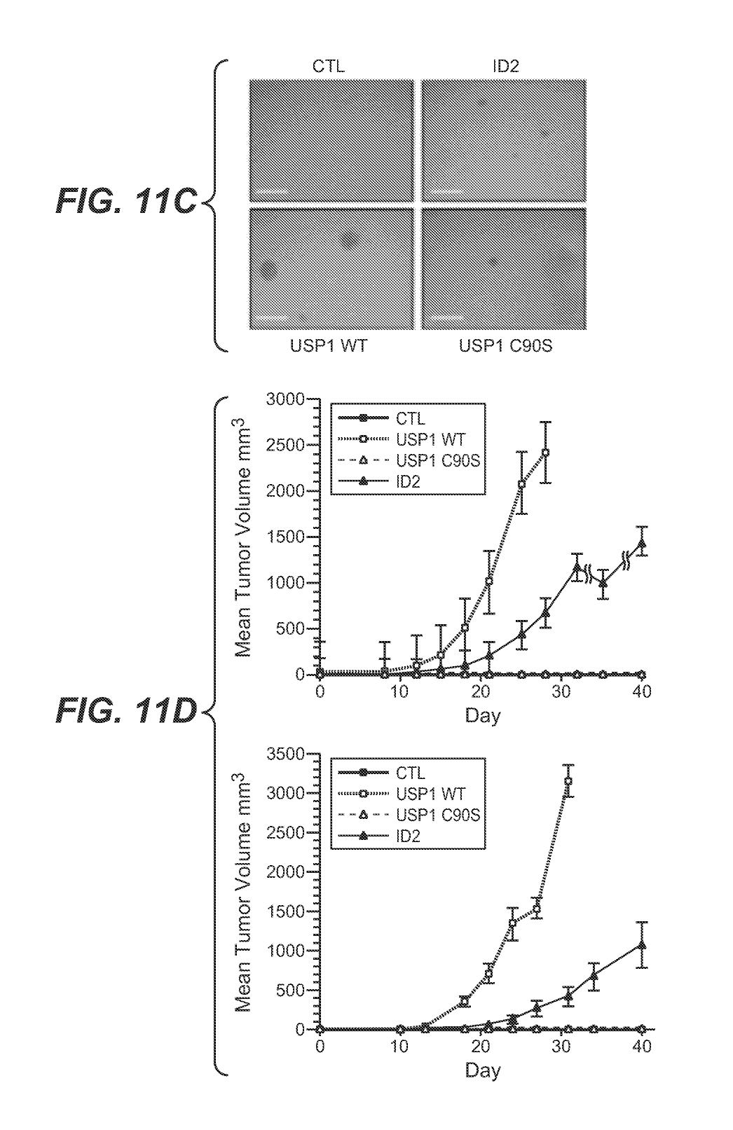

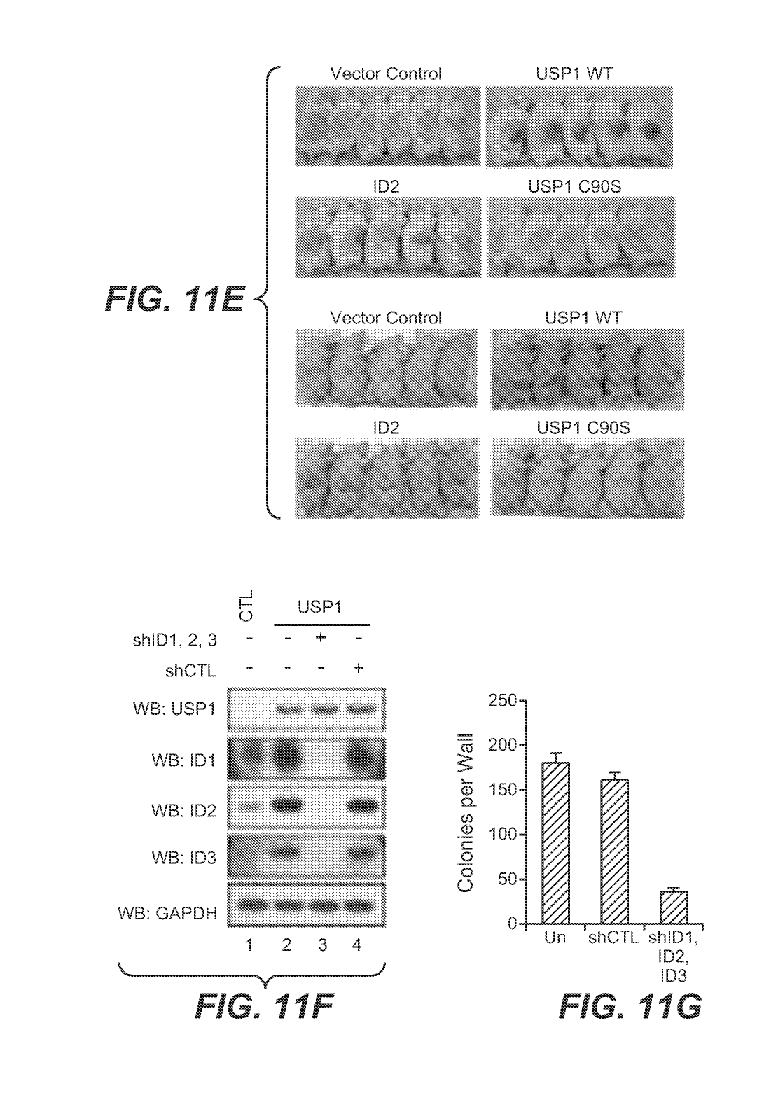

The ability of USP1 to inhibit mesenchymal stem cell differentiation and sustain proliferation of osteosarcoma cell lines suggested that USP1 might promote cell transformation. NIH 3T3 cells were stably transduced with empty vector, ID2, USP1, or USP1 C90S. Wild-type USP1 increased expression of IDs 1-3 (FIG. 11A) and caused anchorage-independent cell proliferation in soft agar (FIG. 11B), which is a classic hallmark of oncogenic transformation (Hanahan and Weinberg, 2000). In contrast, cells transduced with empty vector or USP1 C90S did not grow well in soft agar (FIGS. 11B and 11C). Interestingly, USP1 produced larger and more numerous colonies than ID2 (FIG. 11C), suggesting that stabilization of multiple ID proteins may be more transforming than ID2 overexpression alone.

In vitro observations were recapitulated in vivo when NIH 3T3 cells were implanted subcutaneously into C.B-17 SCID.bg mice. Control cells and cells expressing USP1 C90S failed to produce measurable tumors, whereas cells overexpressing USP1 or ID2 produced measurable tumors as early as 7 days postimplantation (FIG. 11D). Gross visual inspection of tumors at the study endpoint confirmed that cells overexpressing USP1 or ID2 produced aggressive malignancies (FIG. 11E). Similar results were observed in NCr nude mice.

The contribution of the IDs to NIH 3T3 cell transformation by USP1 with Id1, Id2, and Id3 shRNAs was accessed. Suppression of IDs 1-3 (FIG. 11F) blocked colony formation in soft agar (FIG. 11G), indicating that the IDs are essential for USP1 transformation of NIH 3T3 cells.

### USP1 Regulates Bone Development

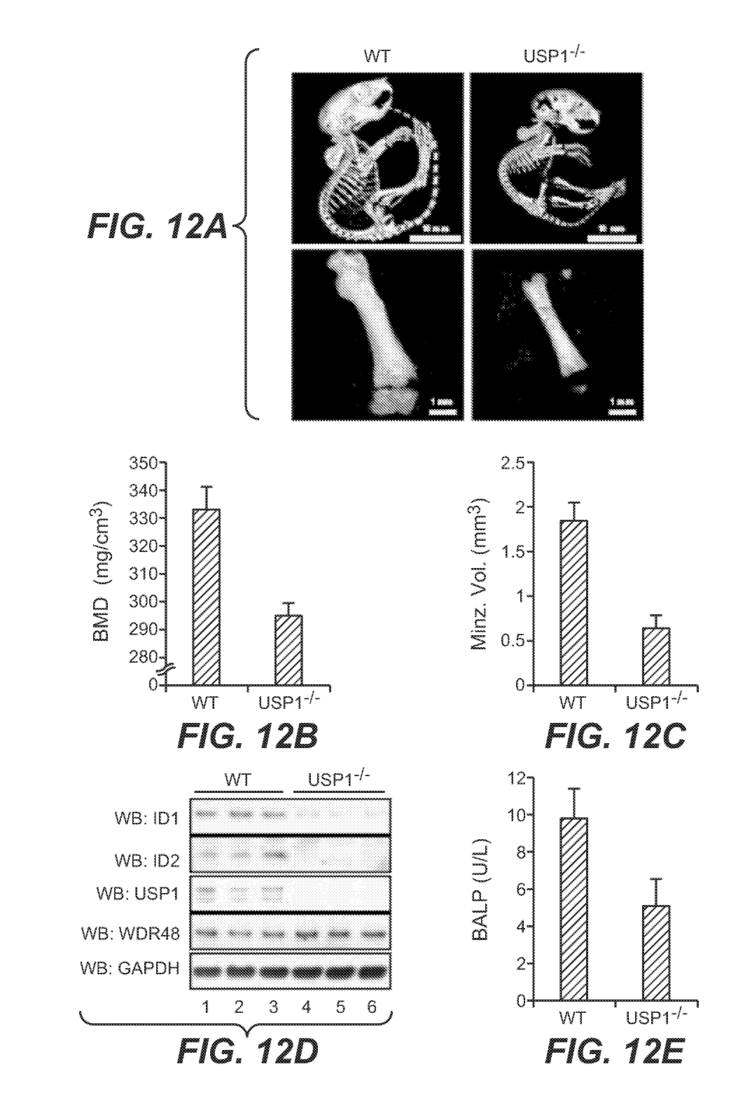

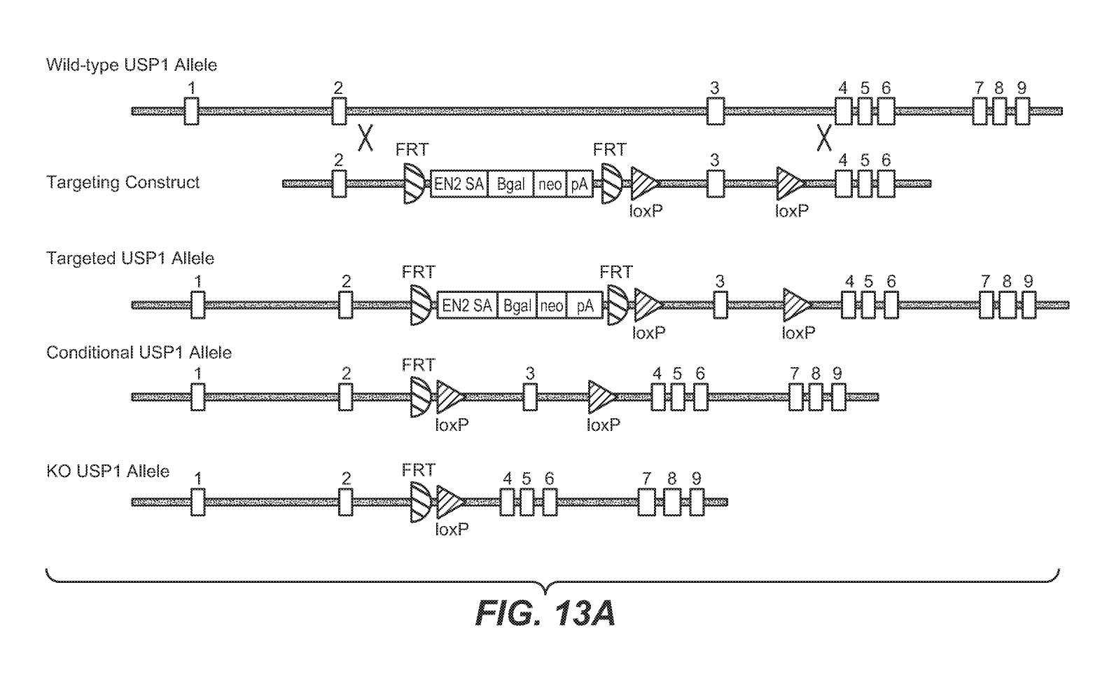

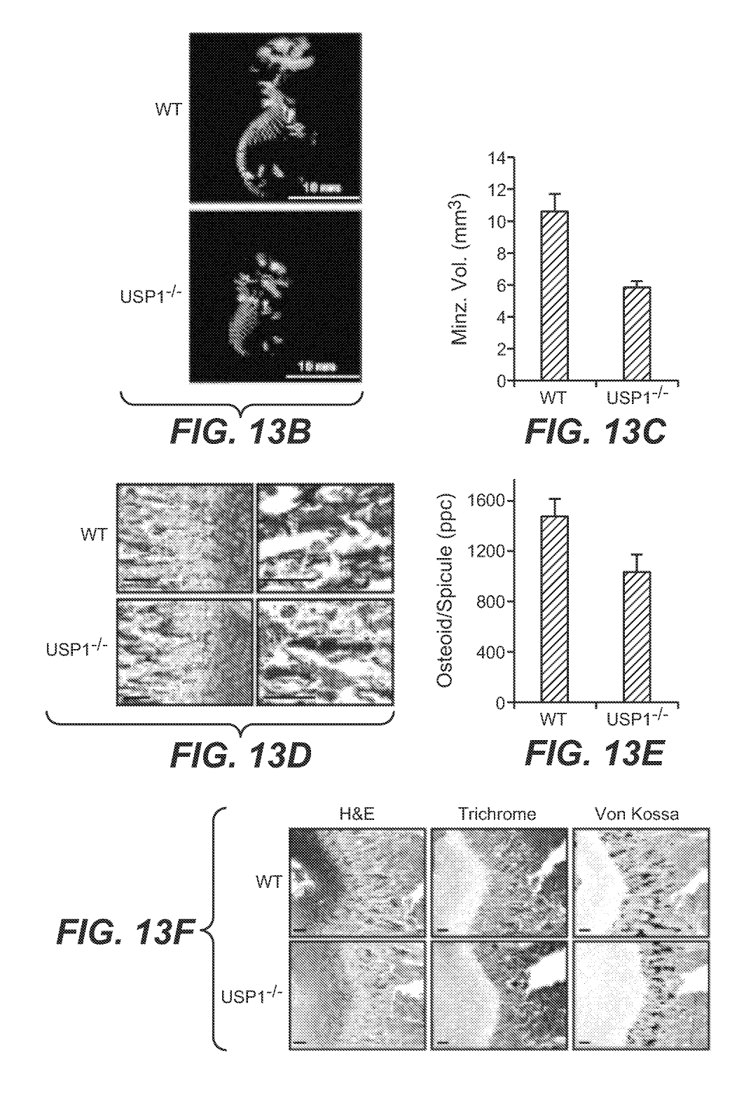

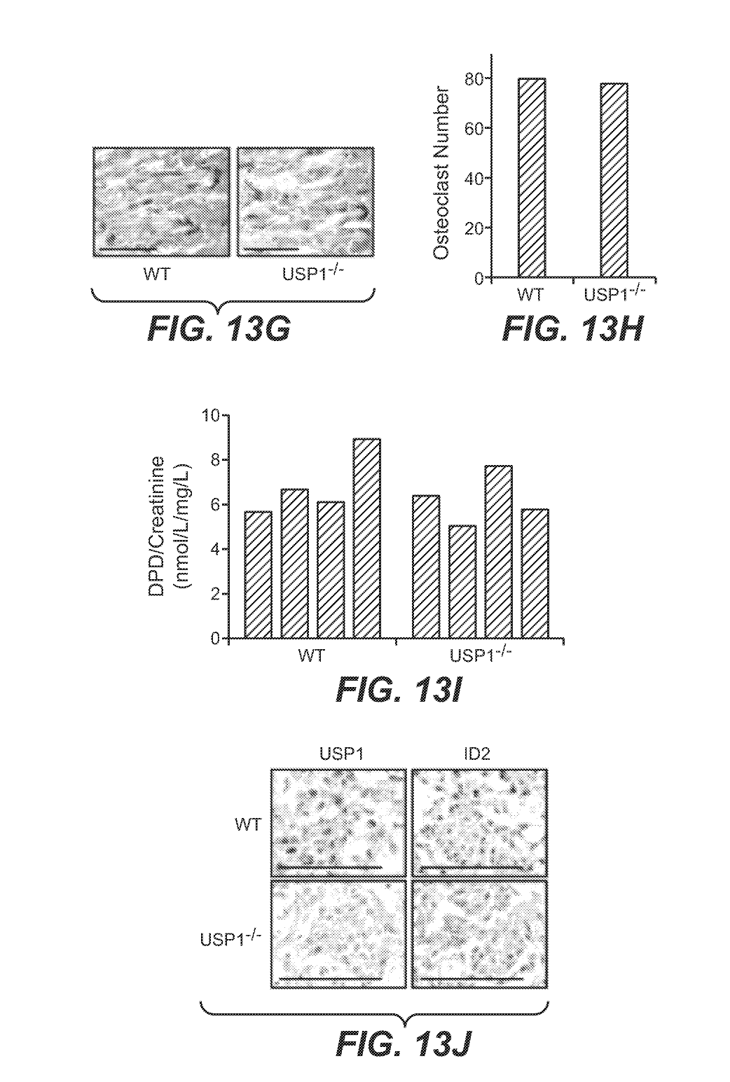

Because USP1 overexpression impaired osteoblastic differentiation of mesenchymal precursors, whereas USP1 loss caused osteoblastic differentiation of osteosarcoma cells, the role of USP1 in regulation of normal bone development was evaluated with USP1 gene-targeted mice. P12 USP1^{-/-} mice were osteopenic with defects in ossification of the cranial and long bones (FIG. 12A). Underdeveloped sternal ribs likely contribute to the lethal cyanotic respiratory failure in USP1^{-/-} pups (Kim et al., 2009). Bone mineral density and volume in USP1^{-/-} neonates and E18.5 embryos were much less than in wild-type littermates (FIGS. 12B and 12C and FIGS. 13B and 13C). Neither FANCD2-nor PCNA-deficient mice exhibit perinatal lethality (Parmar et al., 2010; Roa et al., 2008), excluding destabilization of these USP1 substrates as the primary cause of the perinatal lethality associated with USP1 deficiency.

USP1^{-/-} and USP1^{+/+} femurs contained similar numbers of resting, transitional, proliferating, and hypertrophic chondrocytes, but deposition of osteoid on emergent bone spicules was diminished, suggesting reduced activity of osteoid-depositing osteoblasts (FIGS. 13D-13F). Consistent with a defect in osteoblast function, serum levels of bone alkaline phosphatase (BALP), a marker of systemic osteoblast activity, were reduced in USP1^{-/-} E18.5 embryos (FIG. 12E). USP1 deficiency did not alter osteoclast abundance or activity (FIGS. 13G-13I), excluding increased bone resorption in the USP1^{-/-} mice. Significantly, and in keeping with observations made in osteosarcoma and mesenchymal stem cell cultures, USP1^{-/-} femoral metaphyses contained less ID1 and ID2 than their wild-type counterparts (FIG. 12D and FIG. 13J). These data indicate that the USP1-ID axis regulating differentiation in osteosarcoma is recapitulated n normal skeletal development.

### DISCUSSION

These experiments show that USP1 deubiquitinates and stabilizes ID1, ID2, and ID3, resulting in their increased abundance. Significantly, elevated USP1 protein and mRNA in a subset of primary osteosarcoma tumors correlated with increased ID protein levels. USP1 knockdown in osteosarcoma cells caused ID protein destabilization, p53-independent induction of CDKN1A encoding cyclin-dependent kinase inhibitor (CDKI) p21, and cell cycle arrest. In addition, expression of mesenchymal stem cell markers was decreased and osteogenic differentiation resumed. These data suggest that osteosarcomas, like acute promyelocytic leukemia, may be amenable to differentiation therapies (Soignet et al., 1998). In contrast to USP1 knockdown, USP1 overexpression in primary human mesenchymal stem cells (hMSCs) caused ID protein accumulation and interfered with normal differentiation. Indeed, USP1 promoted transformation in a mesenchymal cell line. Finally, loss of USP1 in gene-targeted mice caused severe osteopenia, which is consistent with a role for USP1 in the mesenchymal lineage. These results strongly suggest that USP1 has oncogenic potential and promotes tumorigenesis through disruption of normal mesenchymal stem cell commitment and differentiation.

In particular, in this study ID stabilization by USP1 was shown to sustain a significant fraction of human osteosarcomas. USP1 was overexpressed frequently in primary osteosarcomas and osteosarcoma cell lines (FIG. 3), and by deubiquitinating the ID proteins (FIGS. 1 and 4), inhibited bHLH-dependent expression of CDKI p21 (FIG. 6) resulting in unchecked cell proliferation (FIG. 8). USP1 overexpression not only was necessary for the proliferation of several osteosarcoma cell lines, it also was sufficient to prevent normal mesenchymal cell differentiation, capturing the cells in a stem-like state (FIG. 10). By contrast, USP1 knockdown in osteosarcoma cell lines reduced expression of mesenchymal stem cell markers and initiated an osteogenic development program (FIG. 8). USP1 deficiency in mice impaired normal osteogenesis and resulted in pronounced osteopenia (FIG. 12). Therefore, it is posit that overexpressed USP1 interferes with mesenchymal stem cell differentiation and thereby fosters the development of malignant mesenchymal cell populations.

USP1 is an ID DUB Overexpressed in Osteosarcoma

The screen for DUBs capable of stabilizing ID2 (FIG. 2) identified both USP1 and USP33, although USP33 was unable to deubiquitinate ID2 (data not shown). USP33 binding ID2 may have precluded ID2 recognition by the proteasome and prevented its degradation. Other DUBs that enhanced ID2 expression in the screen did not appear to interact with ID2 and must influence ID2 abundance indirectly. These DUBs may upregulate ID gene expression, interfere with the ubiquitin-conjugation machinery, or otherwise impair proteasome function. For example USP9X may upregulate ID2 gene expression by deubiquitinating and stabilizing the transcription factor SMAD4 (Dupont et al., 2009).

The mechanism responsible for USP1 overexpression in a subset of osteosarcomas (FIG. 3) is unclear. USP1 mRNA and protein levels correlated strongly implying transcriptional upregulation. Notably, recent CGH analyses found that the USP1 locus 1p31.3 was amplified in 26%-57% of osteosarcoma tumors (Ozaki et al., 2003; Stock et al., 2000).

USP1 Promotes Proliferation Via ID-Mediated Repression of CDKI p21

ID protein stabilization by USP1 was shown to disrupt bHLH dependent p21 expression in osteosarcoma (FIG. 4 and FIG. S3). Thus, USP1 overexpression perturbs normal osteoblast differentiation, which is characterized by p53-independent upregulation of multiple CDKIs (Funato et al., 2001; Kenner et al., 2004; Matsumoto et al., 1998; Yan et al., 1997; Zhang et al., 1997). CDKI function often is compromised in osteosarcomas; CDKN2A/p16INK4a and CDKN2B/p15INK4b gene deletions are common (Miller et al., 1996; Nielsen et al., 1998), as is gene inactivation due to promoter methylation (Oh et al., 2006). In contrast, CDK4, a target of CDKIs, is frequently overexpressed in osteosarcoma due to gene amplification (Ozaki et al., 2003). ID-mediated transcriptional repression of p21 represents an additional oncogenic mechanism in osteosarcoma.

ID protein overexpression has been observed in various human cancers but has been attributed largely to increased ID transcription (Perk et al., 2005). For example ID2 is transcriptionally upregulated by the EWS-Ets translocation in Ewing's sarcoma (Nishimori et al., 2002), which is an osteoid tumor bearing strong resemblance to osteosarcoma. Patients with a disrupted copy of the RB1 gene are strongly sensitized to development of osteosarcoma (Friend et al., 1986), RB being able to sequester and inactivate ID2 (Iavarone et al., 1994; Lasorella et al., 2000). The study reveals an additional mechanism by which ID proteins and, in turn, CDKIs can be dysregulated in osteosarcoma.

ID Proteins Modulate Osteogenic Development of Mesenchymal Precursors

These data also implicate ID proteins in normal osteogenic development. ID2 or USP1 overexpression in mesenchymal stem cells inhibited osteogenic differentiation and promoted retention of mesenchymal stem cell features (FIG. 10). These findings support a recent study describing a role for ID proteins in mesenchymal differentiation (Peng et al., 2004). Intriguingly, Id1/Id3 compound heterozygous mutant mice display calvarial defects and reduced osteoblast outgrowth (Maeda et al., 2004), suggestive of a mesenchymal proliferation defect. It is unknown if additional Id2 deficiency would exacerbate this phenotype due to early lethality. Restricting Id gene deletion to the mesenchymal lineage may prove informative.

The osteopenia that occurs in USP1^{-/-} mice is consistent with the phenotype predicted by USP1 knockdown in osteosarcoma and USP1 overexpression in primary hMSCs (FIG. 12 and FIG. 13). In each setting these data suggest the USP1-ID axis inhibits lineage commitment. An independent USP1-deficient mouse strain also demonstrated runting and perinatal lethality (Kim et al., 2009). Mice lacking multiple Id genes die early in embryogenesis (Lyden et al., 1999), which could indicate that an additional DUB regulates ID protein stability in early development, or that other DUBs can compensate for the absence of USP1.

Recent studies suggest that the bHLH proteins inhibited by IDs 1-3 during osteogenesis may belong to the Hey/Hes family. Hey1 overexpression promoted osteoblastic differentiation, whereas Hey1 knockdown inhibited it (Sharff et al., 2009). Similarly, Hes1 overexpression promoted osteocommitment (Suh et al., 2008). It is possible that multiple bHLH transcription factors act in parallel to promote osteoblast development. USP 1 and ID proteins would be positioned to broadly restrain bHLH-driven commitment signals engaged during differentiation of mesenchymal stem cells. Based on these studies, it is proposed that USP1 belongs to an emerging set of caulo-oncogenes that promotes tumorigenesis through subversion of normal stem (Latin “caulo”) cell biology.

A consequence of these findings, one that has significant therapeutic ramifications, is that inhibition of USP1 protease activity should institute a differentiation program in malignant osteosarcoma leading to a precipitous decline in proliferative capacity and potential reversal of the transformed phenotype. Targeting USP1 would be expected to impact all USP1 substrates including FANCD2, but this may be beneficial because defective DNA repair in tumor cells lacking a normal p53 checkpoint is predicted to sensitize them to crosslinking chemotherapeutic agents or PARP inhibitors (D'Andrea, 2010). Differentiation treatments for cancer, as evidenced by the spectacular success of arsenic as a differentiation therapy for acute promyelocytic leukemia, provide an exciting option for the effective treatment of previously lethal cancers. Targeting USP1 may provide such an opportunity for osteogenic sarcoma.

Sequences

USP1
(SEQ ID NO: 1)
mpgvipsesn glsrgspskk nrlslkffqk ketkraldft
dsqeneekas eyraseidqv vpaaqsspin cekrenllpf
vglnnlgntc ylnsilqvly fcpgfksgvk hlfniisrkk
ealkdeanqk dkgnckedsl asyelicslq sliisveqlq
asfllnpeky tdelatqprr llntlrelnp myegylqhda
qevlqcilgn iqetcqllkk eevknvaelp tkveeiphpk
eemnginsie mdsmrhsedf keklpkgngk rksdtefgnm
kkkvklskeh qsleenqrqt rskrkatsdt lesppkiipk
yisenesprp sqkksrvkin wlksatkqps ilskfcslgk
ittnqgvkgq skenecdpee dlgkcesdnt tngcglespg
ntvtpvnvne vkpinkgeeq igfelveklf qgqlvlrtrc
leceslterr edfqdisvpv qedelskvee sseispepkt
emktlrwais qfasverivg edkyfcench hyteaersll
fdkmpeviti hlkcfaasgl efdcygggls kintplltpl
klsleewstk ptndsyglfa vvmhsgitis sghytasvkv
tdlnsleldk gnfvvdqmce igkpeplnee eargvvenyn
deevsirvgg ntqpskvlnk knveaigllg gqkskadyel
ynkasnpdkv astafaenrn setsdttgth esdrnkessd
qtginisgfe nkisyvvqsl keyegkwllf ddsevkvtee
kdflnslsps tsptstpyll fykkl
UAF
(SEQ ID NO: 40)
maahhrqnta grrkvqvsyv irdevekynr ngvnalqldp
alnrlftagr dsiiriwsvn qhkqdpyias mehhtdwvnd
ivlccngktl isassdttvk vwnahkgfcm stlrthkdyv
kalayakdke lvasagldrq iflwdvntlt altasnntvt
tsslsgnkds iyslamnqlg tiivsgstek vlrvwdprtc
aklmklkght dnvkalllnr dgtqclsgss dgtirlwslg
qqrciatyrv hdegvwalqv ndafthvysg grdrkiyctd
lrnpdirvli ceekapvlkm eldrsadppp aiwvattkst
vnkwtlkgih nfrasgdydn dctnpitplc tqpdqvikgg
asiiqchiln dkrhiltkdt nnnvaywdvl kackvedlgk
vdfedeikkr fkmvyvpnwf svdlktgmlt itldesdcfa
awvsakdagf sspdgsdpkl nlgglllqal leywprthvn
pmdeeenevn hvngeqenrv qkgngyfqvp phtpvifgea
ggrtlfrllc rdsggetesm llnetvpqwv iditvdknmp
kfnkipfylq phassgaktl kkdrlsasdm lqvrkvmehv
yekiinldne sqttsssnne kpgeqekeed iavlaeekie
llcqdqvldp nmdlrtvkhf iwksggdltl hyrqkst
ID1 isoform a
(SEQ ID NO: 2)
mkvasgstat aaagpscalk agktasgage vvrclseqsv
aisrcaggag arlpalldeq qvnvllydmn gcysrlkelv
ptlpqnrkvs kveilqhvid yirdlqleln sesevgtpgg
rglpvrapls tlngeisalt aeaacvpadd rilcr
ID1 isoform b
(SEQ ID NO: 3)
mkvasgstat aaagpscalk agktasgage vvrclseqsv
aisrcaggag arlpalldeq qvnvllydmn gcysrlkelv
ptlpqnrkvs kveilqhvid yirdlqleln sesevgtpgg
rglpvrapls tlngeisalt aevrsrsdh
ID2
(SEQ ID NO: 4)
mkafspvrsv rknslsdhsl gisrsktpvd dpmsllynmn
dcysklkelv psipqnkkvs kmeilqhvid yildlqiald
shptivslhh qrpgqnqasr tplttlntdi silslqasef
pselmsndsk alcg
ID3
(SEQ ID NO: 5)
mkalspvrgc yeavcclser slaiargrgk gpaaeeplsl
lddmnhcysr lrelvpgvpr gtqlsqveil qrvidyildl
qvvlaepapg ppdgphlpiq taeltpelvi sndkrsfch

### PARTIAL LIST OF REFERENCES

Bounpheng, M. A. et al. (1999). FASEB J. 13, 2257-2264.
Ciarapica, R. et al. (2009). Oncogene 28, 1881-1891.
Cohn, M. A. et al. (2007). Mol. Cell 28, 786-797.
D'Andrea, A. D. (2010). N. Engl. J. Med. 362, 1909-1919.
Di Fiore, R. et al. (2009). J. Cell. Physiol. 219, 301-313.
Dupont, S. et al. (2009). Cell 136, 123-135.
Friend, S. H. et al. (1986). Nature 323, 643-646.
Funato, N. et al. (2001). Mol. Cell. Biol. 21, 7416-7428.
Hanahan, D., and Weinberg, R. A. (2000). Cell 100, 57-70.
Huang, T. T. et al. (2006). Nat. Cell Biol. 8, 339-347.
Iavarone, A. et al. (1994). Genes Dev. 8, 1270-1284.
Kastan, M. B. et al. (1991). Cancer Res. 51, 6304-6311.
Kenner, L. et al. (2004). J. Cell Biol. 164, 613-623.
Kim, D. et al. (1999). Mol. Cell. Biol. 19, 8240-8253.
Kim, J. M. et al. (2009). Dev. Cell 16, 314-320.
Lasorella, A. et al. (2000). Nature 407, 592-598.
Lasorella, A. et al. (2001). Oncogene 20, 8326-8333.
Lasorella, A. et al. (2006). Nature 442, 471-474.
Lassar, A. B. et al. (1991). Cell 66, 305-315.
Luo, X. et al. (2008). Lab. Invest. 88, 1264-1277.
Lyden, D. et al. (1999). Nature 401, 670-677.
Maeda, Y. et al. (2004). J. Cell. Biochem. 93, 337-344.
Massari, M. E., and Murre, C. (2000). Mol. Cell. Biol. 20, 429-440.
Matsumoto, T. et al. (1998). Cancer Lett. 129, 61-68.
Miller, C. W. et al. (1996). Cancer Genet. Cytogenet. 86, 136-142.
Nielsen, G. P. et al. (1998). Am. J. Pathol. 153, 159-163.
Nijman, S. M. et al. (2005). Mol. Cell 17, 331-339.
Nishimori, H. et al. (2002). Oncogene 21, 8302-8309.
Oestergaard, V. H. et al. (2007). Mol. Cell 28, 798-809.
Oh, J. H. et al. (2006). Clin. Orthop. Relat. Res. 442, 216-222.
Ozaki, T. et al. (2003). Cancer Genet. Cytogenet. 140, 145-152.
Palombella, V. J. et al. (1994). Cell 78, 773-785.
Parmar, K. et al. (2010). Stem Cells 28, 1186-1195.
Peng, Y. et al. (2003). J. Cell. Biochem. 90, 1149-1165.
Peng, Y. et al. (2004). J. Biol. Chem. 279, 32941-32949.
Perk, J. et al. (2005). Nat. Rev. Cancer 5, 603-614.
Polyak, K. et al. (1996). Genes Dev. 10, 1945-1952.
Prabhu, S. et al. (1997). Mol. Cell. Biol. 17, 5888-5896.
Roa, S. et al. (2008). Proc. Natl. Acad. Sci. USA 105, 16248-16253.
Rogakou, E. P. et al. (1999). J. Cell Biol. 146, 905-916.
Sharff, K. A. et al. (2009). J. Biol. Chem. 284, 649-659.
Soignet, S. L. et al. (1998). N. Engl. J. Med. 339, 1341-1348.
Stock, C. et al. (2000). Genes Chromosomes Cancer 28, 329-336.
Suh, J. H. et al. (2008). Biochem. Biophys. Res. Commun. 367, 97-102.
Tang, N. et al. (2008). Clin. Orthop. Relat. Res. 466, 2114-2130.
Thiery, J. P. et al. (2009). Cell 139, 871-890.
Tupler, R. et al. (2001). Nature 409, 832-833.
Weintraub, H. et al. (1994). Genes Dev. 8, 2203-2211.
Yan, Y. et al. (1997). Genes Dev. 11, 973-983.
Yokota, Y., and Mori, S. (2002). J. Cell. Physiol. 190, 21-28.
Zhang, P. et al. (1997). Nature 387, 151-158.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of screening for and/or identifying an USP1 antagonist, UAF1 antagonist, and/or an ID antagonist which promotes a change in cell fate said method comprising: comparing (i) a reference cell fate, wherein the reference cell fate is the cell fate of a reference cell with (ii) a candidate cell fate, wherein the candidate cell fate is the cell fate of the reference cell in the presence of an USP1 candidate antagonist, UAF1 candidate antagonist, and/or an ID candidate antagonist, wherein the USP1 candidate antagonist binds USP1, wherein the UAF1 candidate antagonist binds UAF1, and/or the ID candidate antagonist binds ID, whereby a difference in cell fate between the reference cell fate and the candidate cell fate identifies the USP1 candidate antagonist and/or the ID candidate antagonist as promoting a change in cell fate.

2. The method of claim 1, wherein the USP1 candidate antagonist, UAF1 candidate antagonist, and/or the ID candidate antagonist is USP1 candidate antagonist.

3. The method of claim 1, wherein the USP1 candidate antagonist, UAF1 candidate antagonist, and/or the ID candidate antagonist is ID candidate antagonist.

4. The method of claim 3, wherein the ID candidate antagonist is an ID1 candidate antagonist, an ID2 candidate antagonist, and/or an ID3 candidate antagonist.

5. The method of claim 1, wherein the USP1 candidate antagonist, UAF1 antagonist, and/or the ID candidate antagonist is UAF1 candidate antagonist.

6. The method of claim 1, wherein the reference cell fate is a stem cell fate.

7. The method of claim 6, wherein the stem cell fate is a mesenchymal stem cell fate.

8. The method claim 1, wherein the candidate cell fate is an osteoblast cell fate, chondrocyte cell fate, or adipocyte cell fate.

9. The method of claim 8, wherein the candidate cell fate is an osteoblast cell fate.

10. (canceled)

11. A method of promoting a change in cell fate of a cell comprising contacting the cell with an effective amount of USP1 antagonist, UAF1 antagonist, and/or an ID antagonist.

12.-13. (canceled)

14. A method of treating a disease or disorder comprising administering to an individual an effective amount of an USP1 antagonist, UAF1 antagonist, and/or an ID antagonist.

15. The method of claim 14, wherein the individual is selected for the treatment based upon elevated expression levels of one or more genes selected from the group consisting of CD90, CD105, CD106, USP1, UAF1, and ID (e.g., ID1, ID2, or ID3) (e.g., compared to an internal reference (e.g., CD144)) or the individual is not selected for the treatment based upon low expression levels of one or more genes selected from the group consisting of CD90, CD105, CD106, USP1, UAF1, and ID (e.g., ID1, ID2, or ID3) (e.g., compared to an internal reference (e.g., CD144)).

16.-17. (canceled)

18. The method claim 14, wherein the USP1 antagonist, UAF1 antagonist, and/or an ID antagonist induces cell cycle arrest.

19. The method claim 14, wherein the USP1 antagonist, UAF1 antagonist, and/or an ID antagonist is capable of promoting a change in cell fate.

20.-22. (canceled)

23. The method claim 14, wherein the disease or disorder comprises a cell with a stem cell fate (e.g., mesenchymal stem cell fate).

24. The method of claim 11, wherein the cell expresses one or more genes selected from the group consisting of CD90, CD105, CD106, USP1, UAF1, and ID (e.g., ID1, ID2, or ID3).

25. (canceled)

26. The method claim 11, wherein the cell does not significantly express (e.g., does not express or expresses at low levels compared to an internal reference (e.g., CD144)) one or more genes selected from the group consisting of p21, RUNX2, OSTERIX, SPARC/OSTEONECTIN, SPP1/OSTEOPONTIN, BGLAP/OSTEOCALCIN, and alkaline phosphatase (ALP).

27. The method claim 14, wherein the disease or disorder is cancer.

28. The method of claim 27, wherein the cancer is osteosarcoma.

29. The method of claim 27, wherein the cancer expresses one or more genes selected from the group consisting of CD90, CD105, CD106, USP1, UAF1, and ID (e.g., ID1, ID2, or ID3).

30. The method of claim 29, wherein expression levels of one or more genes is elevated compared to an internal reference (e.g., CD144).

31.-39. (canceled)


SEQUENCE LISTING (60 sequences)

SEQ ID NO: 1 - protein, 785 aa, Homo sapiens
MPGVIPSESNGLSRGSPSKKNRLSLKFFQKKETKRALDFTDSQENEEKASEYRASEIDQVVPAAQSSPINCEKRENLLPFVGLNNLGNTCYLNSILQVLYFCPGFKSGVKHLFNIISRKKEALKDEANQKDKGNCKEDSLASYELICSLQSLIISVEQLQASFLLNPEKYTDELATQPRRLLNTLRELNPMYEGYLQHDAQEVLQCILGNIQETCQLLKKEEVKNVAELPTKVEEIPHPKEEMNGINSIEMDSMRHSEDFKEKLPKGNGKRKSDTEFGNMKKKVKLSKEHQSLEENQRQTRSKRKATSDTLESPPKIIPKYISENESPRPSQKKSRVKINWLKSATKQPSILSKFCSLGKITTNQGVKGQSKENECDPEEDLGKCESDNTTNGCGLESPGNTVTPVNVNEVKPINKGEEQIGFELVEKLFQGQLVLRTRCLECESLTERREDFQDISVPVQEDELSKVEESSEISPEPKTEMKTLRWAISQFASVERIVGEDKYFCENCHHYTEAERSLLFDKMPEVITIHLKCFAASGLEFDCYGGGLSKINTPLLTPLKLSLEEWSTKPTNDSYGLFAVVMHSGITISSGHYTASVKVTDLNSLELDKGNFVVDQMCEIGKPEPLNEEEARGVVENYNDEEVSIRVGGNTQPSKVLNKKNVEAIGLLGGQKSKADYELYNKASNPDKVASTAFAENRNSETSDTTGTHESDRNKESSDQTGINISGFENKISYVVQSLKEYEGKWLLFDDSEVKVTEEKDFLNSLSPSTSPTSTPYLLFYKKL

SEQ ID NO: 2 - protein, 155 aa, Homo sapiens
MKVASGSTATAAAGPSCALKAGKTASGAGEVVRCLSEQSVAISRCAGGAGARLPALLDEQQVNVLLYDMNGCYSRLKELVPTLPQNRKVSKVEILQHVIDYIRDLQLELNSESEVGTPGGRGLPVRAPLSTLNGEISALTAEAACVPADDRILCR

SEQ ID NO: 3 - protein, 149 aa, Homo sapiens
MKVASGSTATAAAGPSCALKAGKTASGAGEVVRCLSEQSVAISRCAGGAGARLPALLDEQQVNVLLYDMNGCYSRLKELVPTLPQNRKVSKVEILQHVIDYIRDLQLELNSESEVGTPGGRGLPVRAPLSTLNGEISALTAEVRSRSDH

SEQ ID NO: 4 - protein, 134 aa, Homo sapiens
MKAFSPVRSVRKNSLSDHSLGISRSKTPVDDPMSLLYNMNDCYSKLKELVPSIPQNKKVSKMEILQHVIDYILDLQIALDSHPTIVSLHHQRPGQNQASRTPLTTLNTDISILSLQASEFPSELMSNDSKALCG

SEQ ID NO: 5 - protein, 119 aa, Homo sapiens
MKALSPVRGCYEAVCCLSERSLAIARGRGKGPAAEEPLSLLDDMNHCYSRLRELVPGVPRGTQLSQVEILQRVIDYILDLQVVLAEPAPGPPDGPHLPIQTAELTPELVISNDKRSFCH

SEQ ID NO: 6 - DNA, 29 bp, Artificial Sequence
  source: /note=“Description of Artificial Sequence Synthetic oligonucleotide”
tggtggactttccaagatcaacactcctt

SEQ ID NO: 7 - DNA, 29 bp, Artificial Sequence
  source: /note=“Description of Artificial Sequence Synthetic oligonucleotide”
caaggaatccagtgaccaaacaggcatta

SEQ ID NO: 8 - DNA, 29 bp, Artificial Sequence
  source: /note=“Description of Artificial Sequence Synthetic oligonucleotide”
gagattctccagcacgtcatcgactacat

SEQ ID NO: 9 - DNA, 29 bp, Artificial Sequence
  source: /note=“Description of Artificial Sequence Synthetic oligonucleotide”
ccttctgagttaatgtcaaatgacagcaa

SEQ ID NO: 10 - DNA, 29 bp, Artificial Sequence
  source: /note=“Description of Artificial Sequence Synthetic oligonucleotide”
tggtctccttggagaaaggttctgttgcc

SEQ ID NO: 11 - DNA, 29 bp, Artificial Sequence
  source: /note=“Description of Artificial Sequence Synthetic oligonucleotide”
tgaccaccctgacctacggcgtgcagtgc

SEQ ID NO: 12 - DNA, 22 bp, Artificial Sequence
  source: /note=“Description of Artificial Sequence Synthetic oligonucleotide”
aggcaatacttgctatcttaat

SEQ ID NO: 13 - DNA, 22 bp, Artificial Sequence
  source: /note=“Description of Artificial Sequence Synthetic oligonucleotide”
cgcagcacgtcatcgactacat

SEQ ID NO: 14 - DNA, 22 bp, Artificial Sequence
  source: /note=“Description of Artificial Sequence Synthetic oligonucleotide”
cgcaaagtactctgtggctaaa

SEQ ID NO: 15 - DNA, 22 bp, Artificial Sequence
  source: /note=“Description of Artificial Sequence Synthetic oligonucleotide”
cgcagcacgtcatcgattacat

SEQ ID NO: 16 - DNA, 22 bp, Artificial Sequence
  source: /note=“Description of Artificial Sequence Synthetic oligonucleotide”
ctgactgctactccaagctcaa

SEQ ID NO: 17 - DNA, 22 bp, Artificial Sequence
  source: /note=“Description of Artificial Sequence Synthetic oligonucleotide”
cgccctgattatgaactctata

SEQ ID NO: 18 - DNA, 22 bp, Artificial Sequence
  source: /note=“Description of Artificial Sequence Synthetic oligonucleotide”
acctgattatgaactctataat

SEQ ID NO: 19 - DNA, 22 bp, Artificial Sequence
  source: /note=“Description of Artificial Sequence Synthetic oligonucleotide”
cgccctcttcacttaccctgaa

SEQ ID NO: 20 - RNA, 21 nt, Artificial Sequence
  source: /note=“Description of Artificial Sequence Synthetic oligonucleotide”
aauucuccgaacgugucacgu

SEQ ID NO: 21 - DNA, 21 bp, Artificial Sequence
  source: /note=“Description of Artificial Sequence Synthetic oligonucleotide”
cgatggaacttcgactttgtt

SEQ ID NO: 22 - DNA, 21 bp, Artificial Sequence
  source: /note=“Description of Artificial Sequence Synthetic primer”
gccactcagccaaggcgactg

SEQ ID NO: 23 - DNA, 30 bp, Artificial Sequence
  source: /note=“Description of Artificial Sequence Synthetic primer”
cagaatgcctcatactgtccatctctatgc

SEQ ID NO: 24 - DNA, 19 bp, Artificial Sequence
  source: /note=“Description of Artificial Sequence Synthetic primer”
gagctggtgcccaccctgc

SEQ ID NO: 25 - DNA, 20 bp, Artificial Sequence
  source: /note=“Description of Artificial Sequence Synthetic primer”
gatcgtccgcaggaacgcat

SEQ ID NO: 26 - DNA, 27 bp, Artificial Sequence
  source: /note=“Description of Artificial Sequence Synthetic primer”
caagaaggtgagcaagatggaaatcct

SEQ ID NO: 27 - DNA, 31 bp, Artificial Sequence
  source: /note=“Description of Artificial Sequence Synthetic primer”
acagtgctttgctgtcatttgacattaactc

SEQ ID NO: 28 - DNA, 20 bp, Artificial Sequence
  source: /note=“Description of Artificial Sequence Synthetic primer”
gagccgctgagcttgctgga

SEQ ID NO: 29 - DNA, 24 bp, Artificial Sequence
  source: /note=“Description of Artificial Sequence Synthetic primer”
atgacaagttccggagtgagctcg

SEQ ID NO: 30 - DNA, 30 bp, Artificial Sequence
  source: /note=“Description of Artificial Sequence Synthetic primer”
cttggcctgcccaagctctaccttcccacg

SEQ ID NO: 31 - DNA, 30 bp, Artificial Sequence
  source: /note=“Description of Artificial Sequence Synthetic primer”
gggcttcctcttggagaagatcagccggcg

SEQ ID NO: 32 - DNA, 28 bp, Artificial Sequence
  source: /note=“Description of Artificial Sequence Synthetic primer”
atgggactgtggttactgtcatggcggg

SEQ ID NO: 33 - DNA, 38 bp, Artificial Sequence
  source: /note=“Description of Artificial Sequence Synthetic primer”
ctgggttcccgaggtccatctactgtaactttaattgc

SEQ ID NO: 34 - DNA, 27 bp, Artificial Sequence
  source: /note=“Description of Artificial Sequence Synthetic primer”
ctctccatctgcctggctccttgggac

SEQ ID NO: 35 - DNA, 36 bp, Artificial Sequence
  source: /note=“Description of Artificial Sequence Synthetic primer”
cctcaggctatgctaatgattaccctcccttttccc

SEQ ID NO: 36 - DNA, 30 bp, Artificial Sequence
  source: /note=“Description of Artificial Sequence Synthetic primer”
gcaccatgagggcctggatcttctttctcc

SEQ ID NO: 37 - DNA, 26 bp, Artificial Sequence
  source: /note=“Description of Artificial Sequence Synthetic primer”
ggttctggcagggattttccgccacc

SEQ ID NO: 38 - DNA, 18 bp, Artificial Sequence
  source: /note=“Description of Artificial Sequence Synthetic primer”
gtcgacaacggctccggc

SEQ ID NO: 39 - DNA, 20 bp, Artificial Sequence
  source: /note=“Description of Artificial Sequence Synthetic primer”
ggtgtggtgccagattttct

SEQ ID NO: 40 - protein, 677 aa, Homo sapiens
MAAHHRQNTAGRRKVQVSYVIRDEVEKYNRNGVNALQLDPALNRLFTAGRDSIIRIWSVNQHKQDPYIASMEHHTDWVNDIVLCCNGKTLISASSDTTVKVWNAHKGFCMSTLRTHKDYVKALAYAKDKELVASAGLDRQIFLWDVNTLTALTASNNTVTTSSLSGNKDSIYSLAMNQLGTIIVSGSTEKVLRVWDPRTCAKLMKLKGHTDNVKALLLNRDGTQCLSGSSDGTIRLWSLGQQRCIATYRVHDEGVWALQVNDAFTHVYSGGRDRKIYCTDLRNPDIRVLICEEKAPVLKMELDRSADPPPAIWVATTKSTVNKWTLKGIHNFRASGDYDNDCTNPITPLCTQPDQVIKGGASIIQCHILNDKRHILTKDTNNNVAYWDVLKACKVEDLGKVDFEDEIKKRFKMVYVPNWFSVDLKTGMLTITLDESDCFAAWVSAKDAGFSSPDGSDPKLNLGGLLLQALLEYWPRTHVNPMDEEENEVNHVNGEQENRVQKGNGYFQVPPHTPVIFGEAGGRTLFRLLCRDSGGETESMLLNETVPQWVIDITVDKNMPKFNKIPFYLQPHASSGAKTLKKDRLSASDMLQVRKVMEHVYEKIINLDNESQTTSSSNNEKPGEQEKEEDIAVLAEEKIELLCQDQVLDPNMDLRTVKHFIWKSGGDLTLHYRQKST

SEQ ID NO: 41 - protein, 100 aa, Artificial Sequence
  source: /note=“Description of Artificial Sequence Synthetic polypeptide”
QVLTQTPSPVSAAVGGTVTINCQASQSIYNDNDLAWFQQKPGQPPKLLIYDASTLTSGVPSRFKGSGSGTQFTLTISDLDCDDAATYYCAARYSGNIYGF

SEQ ID NO: 42 - protein, 112 aa, Artificial Sequence
  source: /note=“Description of Artificial Sequence Synthetic polypeptide”
QSVEESGGRLVTPGTPLTLTCTVSGIDLSSYAMSWVRQAPGKGLEWIGVIFPSNNVYYASWAKGRFTISKTSTTVDLKITSPTTEDTATYFCASMGAFDSWGPGTLVTVSSG

SEQ ID NO: 43 - protein, 111 aa, Artificial Sequence
  source: /note=“Description of Artificial Sequence Synthetic polypeptide”
AVLTQTPSPVSAAVGGTVSISCQSSQSVWNNNWLSWFQQKPGQPPKLLIYETSKLESGVPSRFKGSGSGTQFTLTISDVQCDDAATYYCLGGYWTTSDNNVFGGGTEVVVK

SEQ ID NO: 44 - protein, 111 aa, Artificial Sequence
  source: /note=“Description of Artificial Sequence Synthetic polypeptide”
QSVEESGGRLVTPGTPLTLTCTASGFSLSNVYIHWVRQAPGKGLEWIGYISDGDTARYATWAKGRFTISKTSSTTVNLKMTSLTTEDTATYFCARQGFNIWGPGTLVTVSL

SEQ ID NO: 45 - protein, 110 aa, Artificial Sequence
  source: /note=“Description of Artificial Sequence Synthetic polypeptide”
AVLTQTPSPVSAAVGGTVTSCQSSQSVYNNNWLSWFQQKSGQPPKLLIYETSKLESGVPSRFKGSGSGTQFTLTIIDVQCDDAATYYCLGGYWTTSDNNIFGGGTEVVVK

SEQ ID NO: 46 - protein, 111 aa, Artificial Sequence
  source: /note=“Description of Artificial Sequence Synthetic polypeptide”
QSVEESGGRLVTPGTPLTLTCTASGFSLSSYYIHWVRQAPGKALEWIGYISDGGTTYYASWAKGRFTISKTSSTTVDLKMTSLTTEDTATYFCARQGFNIWGPGTLVTVSL

SEQ ID NO: 47 - protein, 111 aa, Artificial Sequence; identical to SEQ ID NO: 43
  source: /note=“Description of Artificial Sequence Synthetic polypeptide”

SEQ ID NO: 48 - protein, 111 aa, Artificial Sequence; identical to SEQ ID NO: 44
  source: /note=“Description of Artificial Sequence Synthetic polypeptide”

SEQ ID NO: 49 - protein, 111 aa, Artificial Sequence
  source: /note=“Description of Artificial Sequence Synthetic polypeptide”
AVLTQTPSPVSAAVGGTVTISCQSSQSVYNNNWLSWFQQKSGQPPKLLIYETSKLESGVPSRFKGSGSGTQFTLTIIDVQCDDAATYYCLGGYWSTSDNNIFGGGTEVVVK

SEQ ID NO: 50 - protein, 111 aa, Artificial Sequence; identical to SEQ ID NO: 46
  source: /note=“Description of Artificial Sequence Synthetic polypeptide”

SEQ ID NO: 51 - protein, 596 aa, Artificial Sequence
  source: /note=“Description of Artificial Sequence Synthetic polypeptide”
MSNYSVSLVGPAPWGFRLQGGKDFNMPLTISSLKDGGKAAQANVRIGDVVLSIDGINAQGMTHLEAQNKIKGCTGSLNMTLQRASAAPKPEPVPVQKGEPKEVVKPVPITSPAVSKVTSTNNMAYNKAPRPFGSVSSPKVTSIPSPSSAFTPAHATTSSHASPSPVAAVTPPLFAASGLHANANLSADQSPSALSAGKTAVNVPRQPTVTSVCSETSQELAEGQRRGSQGDSKQQNGPPRKHIVERYTEFYHVPTHSDASKKRLIEDTEDWRPRTGTTQSRSFRILAQITGTEHLKESEADNTKKANNSQEPSPQLASSVASTRSMPESLDSPTSGRPGVTSLTTAAAFKPVGSTGVIKSPSWQRPNQGVPSTGRISNSATYSGSVAPANSALGQTQPSDQDTLVQRAEHIPAGKRTPMCAHCNQVIRGPFLVALGKSWHPEEFNCAHCKNTMAYIGFVEEKGALYCELCYEKFFAPECGRCQRKILGEVINALKQTWHVSCFVCVACGKPIRNNVFHLEDGEPYCETDYYALFGTICHGCEFPIEAGDMFLEALGYTWHDTCFVCSVCCESLEGQTFFSKKDKPLCKKHAHSVNF

SEQ ID NO: 52 - protein, 487 aa, Artificial Sequence
  source: /note=“Description of Artificial Sequence Synthetic polypeptide”
MSNYSVSLVGPAPWGFRLQGGKDFNMPLTISSLKDGGKAAQANVRIGDVVLSIDGINAQGMTHLEAQNKIKGCTGSLNMTLQRASAAPKPEPVPVQKPTVTSVCSETSQELAEGQRRGSQGDSKQQNGPPRKHIVERYTEFYHVPTHSDASKKRLIEDTEDWRPRTGTTQSRSFRILAQITGTEHLKESEADNTKKANNSQEPSPQLASSVASTRSMPESLDSPTSGRPGVTSLTTAAAFKPVGSTGVIKSPSWQRPNQGVPSTGRISNSATYSGSVAPANSALGQTQPSDQDTLVQRAEHIPAGKRTPMCAHCNQVIRGPFLVALGKSWHPEEFNCAHCKNTMAYIGFVEEKGALYCELCYEKFFAPECGRCQRKILGEVINALKQTWHVSCFVCVACGKPIRNNVFHLEDGEPYCETDYYALFGTICHGCEFPIEAGDMFLEALGYTWHDTCFVCSVCCESLEGQTFFSKKDKPLCKKHAHSVNF

SEQ ID NO: 53 - DNA, 21 bp, Artificial Sequence
  source: /note=“Description of Artificial Sequence Synthetic oligonucleotide”
ttggcaagttatgaattgata

SEQ ID NO: 54 - DNA, 21 bp, Artificial Sequence
  source: /note=“Description of Artificial Sequence Synthetic oligonucleotide”
tcggcaatacttgctatctta

SEQ ID NO: 55 - DNA, 19 bp, Artificial Sequence
  source: /note=“Description of Artificial Sequence Synthetic oligonucleotide”
acagttcgcttctacacaa

SEQ ID NO: 56 - DNA, 19 bp, Artificial Sequence
  source: /note=“Description of Artificial Sequence Synthetic oligonucleotide”
gcggtgttcatgatttctt

SEQ ID NO: 57 - DNA, 22 bp, Artificial Sequence
  source: /note=“Description of Artificial Sequence Synthetic oligonucleotide”
caaagcactgtgtgtgggctga

SEQ ID NO: 58 - DNA, 21 bp, Artificial Sequence
  source: /note=“Description of Artificial Sequence Synthetic oligonucleotide”
ccggtcgagactctatcataa

SEQ ID NO: 59 - DNA, 21 bp, Artificial Sequence
  source: /note=“Description of Artificial Sequence Synthetic oligonucleotide”
cacaagcaagatccatatata

SEQ ID NO: 60 - DNA, 19 bp, Artificial Sequence
  source: /note=“Description of Artificial Sequence Synthetic oligonucleotide”
caagcaagatccatatata